(19) United States
(12) Patent Application Publication
(10) Pub. No.: US 2008/0300288 A1
(43) Pub. Date: Dec. 4, 2008

(54) Alkenyldiarylmethanes, Fused Analogs And Syntheses Thereof

(75) Inventors: Mark S. Cushman, West Lafayette, IN (US); Bo-Liang Deng, Sichuan Province (CN)

Correspondence Address: BARNES & THORNBURG LLP, 11 SOUTH MERIDIAN, INDIANAPOLIS, IN, 46204, US

(73) Assignee: PURDUE RESEARCH FOUNDATION, West Lafayette, IN (US)

(21) Appl. No.: 11/993,938

(22) PCT Filed: Jun. 29, 2006
(86) PCT No.: PCT/US2006/025392

§ 371 Date: Dec. 26, 2007

Related U.S. Application Data
(60) Provisional application No. 60/695,570, filed on Jun. 30, 2005.
(60) Provisional application No. 60/729,838, filed on Oct. 25, 2005.

(51) Int. Cl. C07D 261/12; C07D 261/20; C07D 413/02; C07D 413/14; C07C 69/76; A61K 31/42; A61K 31/216; A61P 31/12

(52) U.S. Cl. 514/375; 548/231; 514/376; 514/532; 560/104; 560/101; 548/221

(57) ABSTRACT

Non-nucleoside inhibitors of HIV-1 reverse transcriptase are described. Such inhibitors may be used as part of a combination therapy to treat HIV infection. Compounds described herein exhibit antiviral potency. In addition, compounds described herein exhibit metabolic stability. Also described herein are processes for preparing Non-nucleoside inhibitors of HIV-1 reverse transcriptase.

### CROSS REFERENCE TO RELATED APPLICATIONS

This application claims the benefit under 35 U.S.C. § 119 (e) of U.S. Provisional Application Ser. No. 60/695,570, filed Jun. 30, 2005, and U.S. Provisional Application Ser. No. 60/729,838, filed Oct. 25, 2005, the disclosures of which are hereby incorporated herein by reference.

### TECHNICAL FIELD

The invention described herein relates to compositions useful for treating viral diseases. In particular, the compounds described herein are useful for treating acquired immunodeficiency syndrome (AIDS), and/or human immunodeficiency virus (HIV) infection.

### BACKGROUND

Acquired immunodeficiency syndrome (AIDS) is responsible for millions of deaths worldwide. In addition, tens of millions of persons are living with HIV. The reverse transcriptase (RT) of the human immunodeficiency virus type 1 (HIV-1) plays an essential and central role in the viral replication cycle by conversion of the single-stranded RNA genome of HIV-1 into a double-stranded DNA chain that subsequently is incorporated into the DNA of the infected host cell. HIV-1 RT is a multifunctional heterodimer consisting of a 66-kDa subunit and a 51-kDa subunit that, as a proteolytic product of the p66 subunit, has the same sequence as the corresponding region of p66 subunit but adopts a different conformation.

As an essential viral enzyme, HIV-1 RT is one of the major targets of the antiretroviral drug therapies that are used in the treatment of AIDS. It has been reported that non-nucleoside inhibitors of HIV-1 reverse transcriptase (NNRTIs) inhibit the enzyme by occupation of an induced allosteric binding site very close to the active site. See generally, De Clercq, E. Non-Nucleoside Reverse Transcriptase Inhibitors (NNRTIs): Past, Present, and Future. Chem. Biodiversity 2004, 1, 44-64; Esnouf, R.; Ren, J.; Ross, C.; Jones, Y.; Stammers, D.; Stuart, D. Mechanism of Inhibition of Reverse Transcriptase by Nonnucleoside Inhibitors. Nat. Struct. Biol. 1995, 2, 303-308. However, the emergence of resistant HIV viral strains is a limitation for all therapeutic classes. Cross-resistance has been reported among the approved drugs nevirapine, delavirdine, and efavirenz. Therefore, the development of new NNRTIs with more favorable side effect profiles and improved characteristics influencing drug compliance is needed for future management of HIV infection.

### SUMMARY OF THE INVENTION

Described herein are compounds useful for treating viral diseases, and methods for treating viral diseases. In addition, described herein are processes for preparing the compounds useful for treating viral diseases.

In one embodiment, alkenyldiarylmethanes having the general formula (I) are described

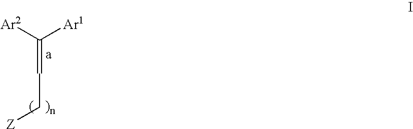

wherein

Ar¹ and Ar² are each independently selected from optionally substituted monocyclic and bicyclic aryls;

double bond a is an E-double bond or a Z-double bond;

n is an integer in the range from 1 to about 5; and

Z is a carboxylic acid derivative or an analog thereof.

In one illustrative aspect of the compounds described herein, the groups Ar¹ and Ar² are the same. In another aspect, the groups Ar¹ ands Ar² are different. In another aspect, the double bond in formula I has the E-geometry. In another aspect, the double bond in formula I has the Z-geometry. In another aspect, the group Z is an ester, such as an optionally substituted alkyl or optionally substituted aryl ester. In another aspect, when Z is a methyl ester, the groups Ar¹ and Ar² are different. In another aspect, the group Z is a cyclic analog of a carboxylic acid, such as an oxazolidinone, and the like. In another aspect, the groups Ar¹ and Ar² are different and the group Z is a cyclic analog of a carboxylic acid, such as an oxazolidinone, and the like. In another aspect, the integer n is 2 or 3.

In another embodiment, alkenyldiarylmethanes having the general formula (II) are described

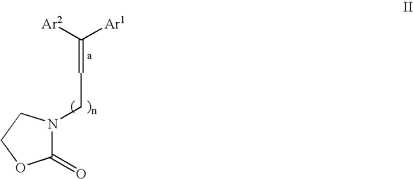

wherein

Ar¹ and Ar² are each independently selected from optionally substituted monocyclic and bicyclic aryls;

double bond a is an E-double bond or a Z-double bond; and

n is an integer in the range from 1 to about 5.

In another embodiment, alkenyldiarylmethanes having the general formulae (III) are described

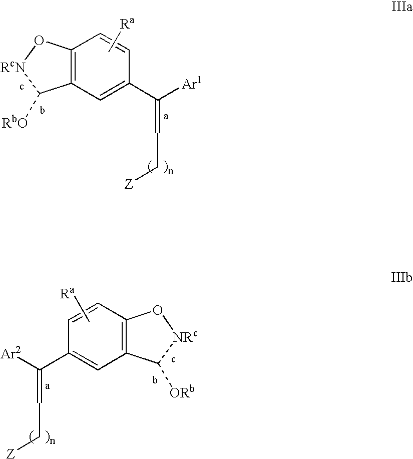

wherein

Ar¹ and Ar² are selected from optionally substituted monocyclic and bicyclic aryls;

double bond a is an E-double bond or a Z-double bond;

n is an integer in the range from 1 to about 5;

Z is a carboxylic acid derivative or an analog thereof; and

R^{a} represents 1, 2, or 3 substituents each independently selected from the group consisting of halo, alkyl, alkoxy, haloalkyl, haloalkoxy, alkylthio, hydroxy, nitro, carboxylate and derivatives thereof, cyano, carbamoyl, carboxamido, amino, alkylamino, dialkylamino, alkylalkylamino, sulfonamide, and alkylsulfonylamino; and

one of bond b or bond c is a double bond, and the other of bond b or bond c is a single bond; and R^{b} and R^{c} are each an optionally substituted alkyl; providing that when bond b is a double bond, R^{b} is absent; and when bond c is a double bond, R^{c} is absent.

In another embodiment, alkenyldiarylmethanes having the general formulae (IV) are described

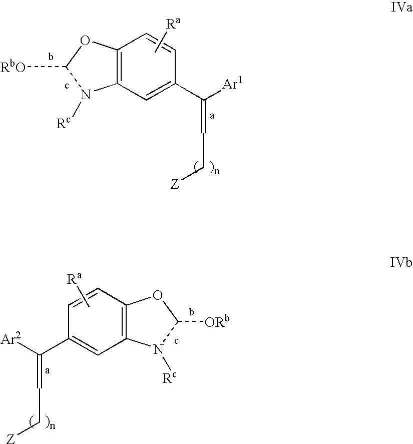

wherein

Ar¹ and Ar² are selected from optionally substituted monocyclic and bicyclic aryls;

double bond a is an E-double bond or a Z-double bond;

n is an integer in the range from 1 to about 5;

Z is a carboxylic acid derivative or an analog thereof; and

R^{a} represents 1, 2, or 3 substituents each independently selected from the group consisting of halo, alkyl, alkoxy, haloalkyl, haloalkoxy, alkylthio, hydroxy, nitro, carboxylate and derivatives thereof, cyano, carbamoyl, carboxamido, amino, alkylamino, dialkylamino, alkylalkylamino, sulfonamide, and alkylsulfonylamino; and

one of bond b or bond c is a double bond, and the other of bond b or bond c is a single bond; and R^{b} and R^{c} are each an optionally substituted alkyl; providing that when bond b is a double bond, R^{b} is absent; and when bond c is a double bond, R^{c} is absent.

In another embodiment, the compounds of formulae I-IV described herein are useful for treating viral diseases, such as acquired immunodeficiency syndrome (AIDS), human immunodeficiency virus (HIV) infection, and the like. In another aspect, the compounds of formulae I-IV described herein are efficacious against viral strains, such as HIV viral strains, that have become resistant to other drugs, including other alkenyldiarylmethanes, azidothymidine (AZT), nevirapine, delavirdine, efavirenz, and the like. In another aspect, the compounds of formulae I-IV described herein have improved metabolic stability, such as improved metabolic stability in plasma as determined by the half-life of the compounds in rat blood plasma. In another aspect, the compounds of formulae I-IV described herein inhibit the cytopathic effect of HIV-1 reverse transcriptase.

In another embodiment, methods for treating viral diseases are described. In one aspect of the methods described herein, the viral disease is attributable to HIV. In another aspect, the viral disease is responsive to enzyme inhibition, such as inhibition of HIV-1 reverse transcriptase. In another aspect, the compounds of formulae I-IV described herein are combined with known or conventional compounds or therapies, such as drug combinations that include one or more of the compounds described herein and other alkenyldiarylmethanes, azidothymidine (AZT), nevirapine, delavirdine, efavirenz, and the like.

In another embodiment, processes for preparing the compounds of formulae I-IV are described. In one aspect, the processes include the step of preparing a sulfonate derivative of an alkyl alcohol, such as a primary alcohol, where the step comprises contacting the alcohol with the corresponding sulfonyl chloride or sulfonyl triflate and an inorganic base, such as potassium or sodium hydroxide, in a solvent including tetrahydrofuran (THF) and water. In another aspect, the processes include the step of preparing a methyl ester or aromatic methyl ether, such as a methyl ether of a phenolic hydroxyl, where the step comprises contacting the corresponding carboxylic acid or aryl alcohol with dimethylsulfate, an inorganic base such as potassium carbonate, sodium hydroxide, and the like, and a phase-transfer catalyst, such as a tetraalkylammonium halide, in a biphasic solvent comprising dichloromethane (DCM) and water.

In another aspect, the processes include the step of preparing a compound of the formula

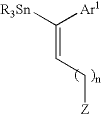

wherein n is an integer in the range from 1 to about 5; Ar¹ is selected from optionally substituted monocyclic and bicyclic aryls; R is an alkyl group, such as n-butyl, and Z is a carboxylic acid derivative or an analog thereof, where the step comprises slowly contacting a dilute solution of a metal catalyst, such as Pd(PPh₃)₄, Pd(PPh₃)₂Cl₂, and the like, and a compound of the formula

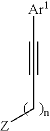

with a trialkyltin hydride. The step proceeds with high regioselectivity and high geometric or stereoselectivity.

In another aspect, the processes include the step of preparing a compound of the formula

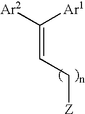

wherein n is an integer in the range from 1 to about 5; Ar¹ and Ar² are each independently selected from optionally substituted monocyclic and bicyclic aryls, and Z is a carboxylic acid derivative or an analog thereof, where the step comprises contacting a solution comprising toluene at reflux, a compound of the formula Ar²-L, where L is a leaving group such as a halo, trialkylstalmnyl, boronyl, and the like, a metal catalyst, such as Pd(P(t-Bu)₃)₂, and the like, CsF, and a compound of the formula

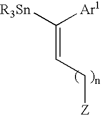

wherein n is an integer in the range from 1 to about 5; Ar¹ is selected from optionally substituted monocyclic and bicyclic aryls; R is an alkyl group, such as n-butyl, and Z is a carboxylic acid derivative or an analog thereof.

It is to be understood that each of these aspects of the various illustrative embodiments described herein may be combined as additional illustrative embodiments. For example, illustrative embodiments of the compounds of formulae I-IV may include those aspects wherein the double bond has the E-geometry and Z is a cyclic analog of a carboxylic acid. In addition, illustrative embodiments of the compounds of formulae I-IV may include those aspects wherein the double bond has the Z-geometry, and Z is a cyclic analog of a carboxylic acid. In addition, illustrative embodiments of the methods described herein may include those aspects wherein the viral disease is AIDS, and the method also includes the step of adding another protease inhibitor, such as AZT. It is to be understood that the additional step may be separate in time from the step of adding a compound of formulae I-IV; or may be contemporaneous or simultaneous. Further, it is to be understood that in the contemporaneous or simultaneous variation the compounds may be combined. In addition, illustrative embodiments of the processes described herein may include those aspects wherein the step of preparing a compound of the formula

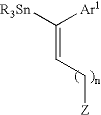

is followed by the step of preparing a compound of the formula

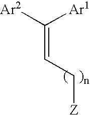

wherein Ar¹, Ar², n, R, and Z are as defined herein.

### DETAILED DESCRIPTION

In one embodiment, alkenyldiarylmethanes having the general formula (I) are described

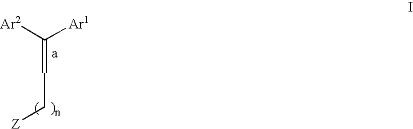

wherein

Ar¹ and Ar² are each independently selected from optionally substituted monocyclic and bicyclic aryls;

double bond a is an E-double bond or a Z-double bond;

n is an integer in the range from 1 to about 5; and

Z is a carboxylic acid derivative or an analog thereof.

In one illustrative aspect of the compounds described herein, the groups Ar¹ and Ar² are the same. In another aspect, the groups Ar¹ ands Ar² are different. In another aspect, the double bond in formula I has the E-geometry. In another aspect, the double bond in formula I has the Z-geometry. In another aspect, the group Z is an ester, such as an optionally substituted alkyl or optionally substituted aryl ester. In another aspect, when Z is a methyl ester, the groups Ar¹ and Ar² are different. In another aspect, when the groups Ar¹ and Ar² are the same, Z is not a methyl ester. In another aspect, when the groups Ar¹ and Ar² are the same, Z is not an alkyl ester. In another aspect, Z is not an alkyl ester. In another aspect, the group Z is a cyclic analog of a carboxylic acid, such as an oxazolidinone, and the like. In another aspect, the groups Ar¹ and Ar² are different and the group Z is a cyclic analog of a carboxylic acid, such as an oxazolidinone, and the like. In another aspect, the integer n is 2 or 3.

In another embodiment, combination therapies are described, wherein the compounds described herein are combined with other known or conventional drugs or therapies. A number of HIV-1 strains containing AZT resistance mutations have shown increased sensitivity to alkenyldiarylmethanes, such as those compounds described herein, indicating a possible therapeutic role for those compounds in combination with AZT. See, Cushman, M.; Casimiro-Garcia, A.; Hejchman, E.; Ruell, J. A.; Huang, M.; Schaeffer, C. A.; Williamson, K.; Rice, W. G.; Buckheit, R. W., Jr. New Alkenyldiarylmethanes with Enhanced Potencies as Anti-HIV Agents Which Act as Non-Nucleoside Reverse Transcriptase Inhibitors J. Med. Chem. 1998, 41, 2076-2089, the disclosure of which is incorporated herein by reference. Alkenyldiarylmethanes have been found to inhibit the cytopathic effect of HIV-1 in cell culture at low nanomolar concentrations, some with EC₅₀ values of about 0.02 μM to about 0.21 μM for inhibition of the cytopathic effect of HIV-1_{RF} in CEM-SS cells, and IC₅₀ values of from about 0.074 μM to about 0.499 μM for HIV-1 RT with rCdG as the template primer.

In another embodiment, it has been observed that when Z in the compounds of formula I is an ester, such as a methyl ester, like those compounds exemplified by 1 and 2,

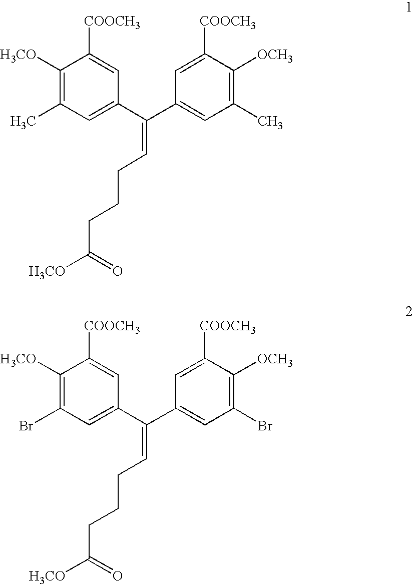

such compounds may be hydrolyzed in vivo, or in vitro such as in blood plasma, to the corresponding, and often biologically inactive acids. Accordingly, in one aspect, the compounds of formula I include more metabolically stable carboxylic acid analogs and derivatives, such as carbamates, cyclic carbamates, oxazolidinones, and the like, such as compounds of formula II

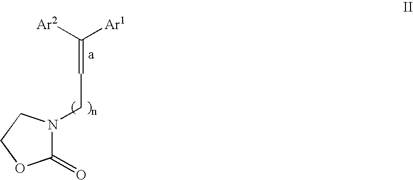

wherein

Ar¹ and Ar² are each independently selected from optionally substituted monocyclic and bicyclic aryls;

double bond a is an E-double bond or a Z-double bond; and

n is an integer in the range from 1 to about 5.

In one illustrative aspect of the compounds described herein, the groups Ar¹ and Ar² are the same. In another aspect, the groups Ar¹ ands Ar² are different. In another aspect, the double bond in formula II has the E-geometry. In another aspect, the double bond in formula II has the Z-geometry. In another aspect, the integer n is 2 or 3. In another aspect, geometrically isomeric compounds 3 and 4 are described. It is appreciated that the cyclic carbamate present in 3 and 4 may be more metabolically stable than the corresponding esters 5 and 6.

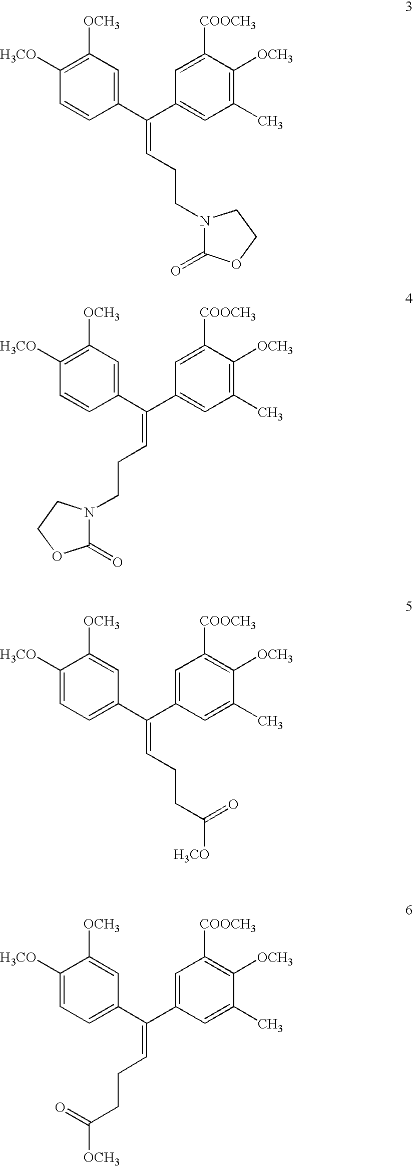

In another embodiment, alkenyldiarylmethanes having the general formulae (III) are described

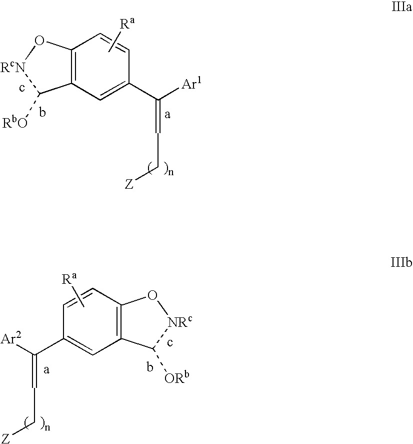

wherein

Ar¹ and Ar² are selected from optionally substituted monocyclic and bicyclic aryls;

double bond a is an E-double bond or a Z-double bond;

n is an integer in the range from 1 to about 5;

Z is a carboxylic acid derivative or an analog thereof; and

R^{a} represents 1, 2, or 3 substituents each independently selected from the group consisting of halo, alkyl, alkoxy, haloalkyl, haloalkoxy, alkylthio, hydroxy, nitro, carboxylate and derivatives thereof, cyano, carbamoyl, carboxamido, amino, alkylamino, dialkylamino, alkylalkylamino, sulfonamide, and alkylsulfonylamino; and

one of bond b or bond c is a double bond, and the other of bond b or bond c is a single bond; and R^{b} and R^{c} are each an optionally substituted alkyl; providing that when bond b is a double bond, R^{b} is absent; and when bond c is a double bond, R^{c} is absent.

In another embodiment, alkenyldiarylmethanes having the general formulae (IV) are described

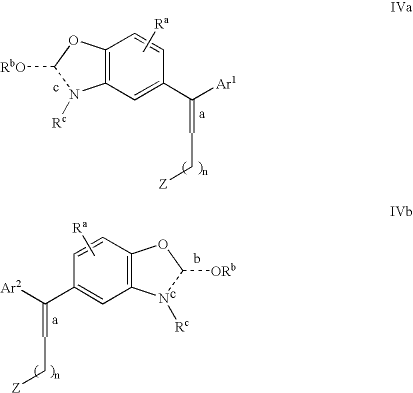

wherein

Ar¹ and Ar² are selected from optionally substituted monocyclic and bicyclic aryls;

double bond a is an E-double bond or a Z-double bond;

n is an integer in the range from 1 to about 5;

Z is a carboxylic acid derivative or an analog thereof; and

R^{a} represents 1, 2, or 3 substituents each independently selected from the group consisting of halo, alkyl, alkoxy, haloalkyl, haloalkoxy, alkylthio, hydroxy, nitro, carboxylate and derivatives thereof, cyano, carbamoyl, carboxamido, amino, alkylamino, dialkylamino, alkylalkylamino, sulfonamide, and alkylsulfonylamino; and

one of bond b or bond c is a double bond, and the other of bond b or bond c is a single bond; and R^{b} and R^{c} are each an optionally substituted alkyl; providing that when bond b is a double bond, R^{b} is absent; and when bond c is a double bond, R^{c} is absent.

In one illustrative aspect of the compounds described herein, the groups Ar¹ and Ar² are the same. In another aspect, the groups Ar¹ ands Ar² are different. In another aspect, the double bond in formulae III-IV has the E-geometry. In another aspect, the double bond in formulae III-IV has the Z-geometry. In another aspect, the group Z is an ester, such as an optionally substituted alkyl or optionally substituted aryl ester. In another aspect, the group Z is a cyclic analog of a carboxylic acid, such as an oxazolidinone, and the like. In another aspect, the groups Ar¹ and Ar² are different and the group Z is a cyclic analog of a carboxylic acid, such as an oxazolidinone, and the like. In another aspect, the integer n is 2 or 3.

As used herein, the term “optionally substituted monocyclic and bicyclic aryls” refers to an aromatic mono or bicyclic ring of carbon atoms, such as phenyl, naphthyl, and the like, and to an aromatic mono or bicyclic ring of carbon atoms and at least one heteroatom selected from nitrogen, oxygen, and sulfur, such as pyridinyl, pyrimidinyl, indolyl, benzoxazolyl, benzisoxazolyl, benzoxazolinonyl, benzisoxazolinonyl, and the like, which may be optionally substituted with one or more independently selected substituents, such halo, alkyl, alkoxy, haloalkyl, haloalkoxy, alkylthio, hydroxy, nitro, carboxylate and derivatives thereof, cyano, carbamoyl, carboxamido, amino, alkylamino, dialkylamino, alkylalkylamino, sulfonamide, and alkylsulfonylamino. In one aspect, substituted monocyclic and substituted bicyclic aryls include those compounds having at least one halo (e.g., fluoro) group, haloalkyl group, or halalkoxy group. In another aspect, substituted monocyclic and substituted bicyclic aryls do not include a carboxylate or derivative thereof. In another aspect, substituted monocyclic and substituted bicyclic aryls include those compounds having a cyano group.

As used herein, the term “alkyl” refers to a saturated monovalent chain of carbon atoms, which may be optionally branched. It is understood that in embodiments that include alkyl, illustrative variations of those embodiments include lower alkyl, such as C₁-C₆, C₁-C₄ alkyl, methyl, ethyl, propyl, 3-methylpentyl, and the like.

As used herein, the terms “alkylamino,” “dialkylamino,” and “alkylalkylamino” refer to amino substituted with alkyl groups, where each alkyl group is independently selected, and illustratively includes methylamino, dimethylamino, methylethylamino, and the like.

In another embodiment, compounds 7-22 are described, which include 5-chloro-2-methoxyphenyl, 3-cyanophenyl, 5-fluoro-2-trifluoromethylphenyl, 3-fluoro-5-trifluoromethylphenyl, and other groups. These compounds were prepared by the processes described herein comprising the steps of Sonogashira and Stille cross-coupling reactions.

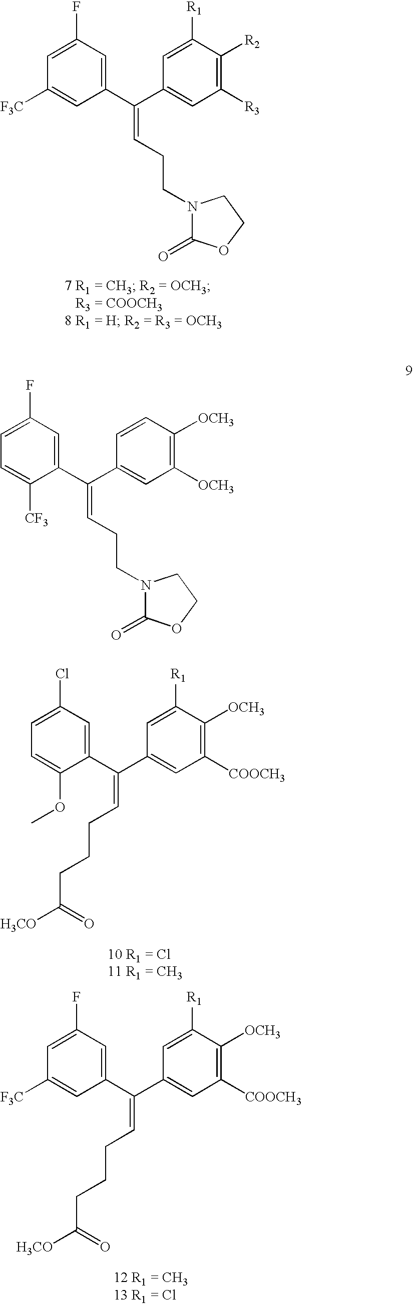

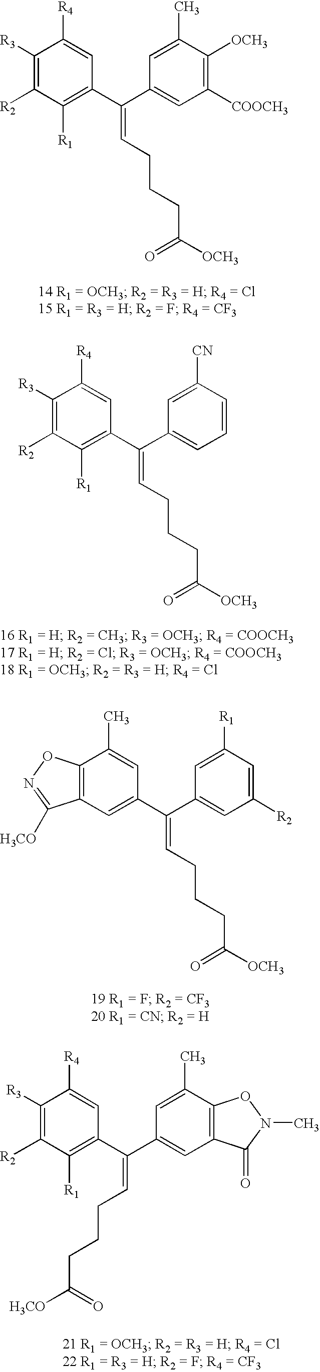

It is appreciated that the isoxazole and isoxazolinone rings of compounds of formula III, illustrated by 19-22 above, may be more metabolically stable than other acyclic substituents. In particular, such isoxazole and isoxazolinone rings may be more metabolically stable than the 4-methoxy-3-methoxycarbonylphenyl substituents found in other compounds of formulae I-IV described herein. Similarly, it is appreciated that the oxazole and oxazolinone rings of compounds of formula IV may be more metabolically stable than other acyclic substituents found in compounds of formulae I-IV.

In another embodiment, processes for preparing compounds of formulae I-IV are described. In one aspect, compounds of formulae I-IV may be prepared by the general synthesis shown in Scheme 1, and illustrated for the preparation of compounds 3 and 4. Commercially available 3-butyn-1-ol (23) was converted to the corresponding tosylate 24, which reacted with 2-oxazolidinone to afford the alkylated intermediate 25. The Sonogashira coupling of the terminal alkyne 25 with the aryl iodide 29 or 33 yielded the disubstituted alkynes 30 or 34. The hydrostannylation of 30 or 34 with tri-n-butyltin hydride in the presence of Pd(PPh₃)₄ afforded the regiochemically and stereochemically defined vinylstannanes 31 or 35, that underwent a Stille cross-coupling with the aryl iodides 33 or 29 to afford the desired target compounds 3 or 4.

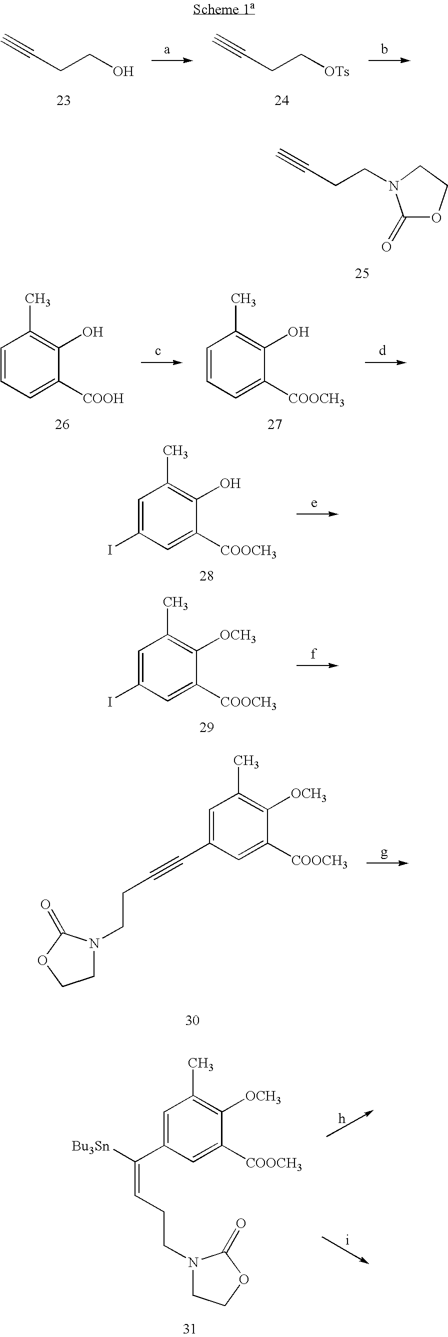

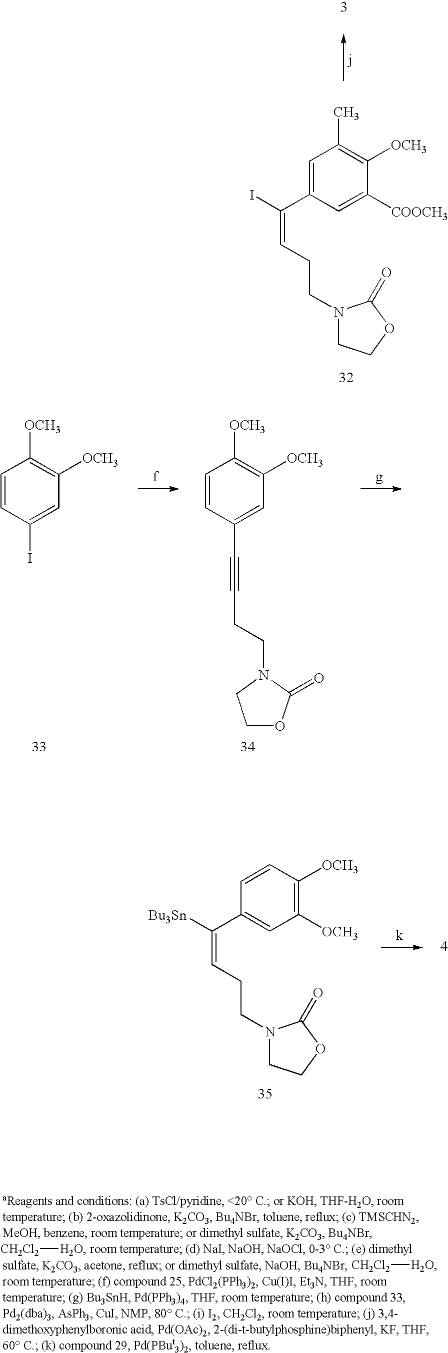

3-Butynyl-1-tosylate (24) may be synthesized in 83% yield from 3-butyn-1-ol (23) with p-toluenesulfonyl chloride in the presence of pyridine. See, Eglinton, G.; Whiting, M. C. Research on Acetylenic Compounds 27. The Preparation and Properties of the Toluene-para-Sulphonates of Acetenic Alcohols J. Chem. Soc. 1950, 3650-3656, the disclosure of which is incorporated herein by reference. It is appreciated that large-scale operations may be impeded by the lack of solvent, difficulty in stirring, and subsequent removal of the pyridine. An alternative synthesis includes the use of inorganic bases such as KOH, NaOH, and the like, and aqueous organic solvents such as water admixed with THF. In one illustrative example, compound 24 was synthesized on a 27-gram scale. This alternative reaction occurs rapidly at room temperature, with a simple work-up procedure.

Oxazolidinones similar to 25 may be prepared by reacting the corresponding bromide or iodide with 1,3-oxazolidin-2-one in the presence of cesium carbonate as the base in acetone. See, Xu, G.; Micklatcher, M.; Silvestri, M.; Hartman, T. L.; Burrier, J.; Osterling, M. C.; Wargo, H.; Turpin, J. A.; Buckheit, R. W., Jr.; Cushman, M. The Biological Effects of Structural Variation at the Meta Position of the Aromatic Rings and at the End of the Alkenyl Chain in the Alkenyldiarylmethane Series of Non-Nucleoside Reverse Transcriptase Inhibitors J. Med. Chem. 2001, 44, 4092-4113, the disclosure of which is incorporated herein by reference. However, in some cases, those reaction conditions may lead to competing elimination reactions. In order to minimize the competitive elimination reaction, the halide leaving group may be changed to a tosylate, which may favor nucleophilic substitution over elimination when a competition between S_{N}2 and E2 processes is involved. It is understood that because 2-oxoazolidinone may be present as the oxazole tautomer under certain conditions, the alkyation may also give rise to N-alkyl derivatives or O-alkyl derivatives; although it has been shown that alkylation of 2-oxoazolidinone with 4-bromo-1-butene results in N-alkylation. In one aspect, a phase-transfer catalyst may be included in the reaction to improve the N versus O selectivity.

In one aspect, the alkylation of 2-oxazolidinone with 3-butynyl-1-tosylate (24) in the presence of tetrabutylammonium bromide in toluene was performed to give 3-but-3-ynyl-1,3-oxazolidin-2-one (25) in 83% yield. Other conditions include the additional use of potassium carbonate as a base, and dichloromethane and water at reflux temperature as the solvent.

5-Iodo-3-methyl-2-methoxybenzoate (29) may be prepared from methyl 2-hydroxy-5-iodo-3-methylbenzoate (28) with dimethyl sulfate and potassium carbonate in refluxing acetone. See, Xu, G.; Loftus, T. L.; Wargo, H.; Turpin, J. A.; Buckheit, R. W.; Cushman, M. Solid Phase Synthesis of the Alkenyldiarylmethane (ADAM) Series of Non-Nucleoside Reverse Transcriptase Inhibitors. J. Org. Chem. 2001, 66, 5958-5964, the disclosure of which is incorporated herein by reference. Similarly, ethyl 2-hydroxy-5-iodo-3-methylbenzoate (28) may be prepared from 3-methyl salicylic acid (26). Alternatively, 3-methyl salicylic acid (26) may be converted into its methyl ester 27 using (trimethylsilyl)diazomethane in a mixture of methanol and benzene. The product 27 may then be iodinated with sodium iodide in the presence of sodium hypochlorite and sodium hydroxide. In some cases, an excess of (trimethylsilyl)diazomethane is added to complete the transformation of 26 to 27.

Alternatively, to avoid the expensive reagent (trimethylsilyl)diazomethane, another embodiment described herein is a synthesis of 29 by converting 3-methyl salicylic acid (26) into its methyl ester 27 using dimethyl sulfate and potassium carbonate in the presence of tetrabutylammonium bromide in a mixture of dichloromethane and water at room temperature. The methyl ester 27 is iodinated with sodium iodide in the presence of sodium hypochlorite and sodium hydroxide to afford methyl 2-hydroxy-5-iodo-3-methylbenzoate (28) in 98% overall yield. 5-Iodo-3-methyl-2-methoxybenzoate (29) is synthesized in 91% yield from methyl 2-hydroxy-5-iodo-3-methylbenzoate (28) with dimethyl sulfate in the presence of tetrabutylammonium bromide and sodium hydroxide in a mixture of dichloromethane and water at room temperature. It is appreciated that this method also has the advantage that, anhydrous solvents, methanol and benzene, may be avoided. It is also appreciated that these reactions have the advantage that they occur rapidly at room temperature, with simple work-up procedures, allowing large-scale runs.

The Sonogashira reaction of compound 25 with iodo compounds 29 or 33 gave alkynes 30 or 34. In one aspect, the hydrostannation of the alkynes 30 or 34 in the presence of Pd(PPh₃)₄ gives the regio- and stereodefined vinylstannane 31 or 35. In another aspect, those conditions gave low conversions. It is appreciated that a general problem may be that the activated Pd—H intermediate Bu₃Sn—Pd—H can proceed down an alternate path by irreversible generation of H₂, dimerization of the stannane, and precipitation of the Pd as palladium black. Alternative conditions include using Pd(PPh₃)₂Cl₂ as the catalyst and employing slow addition of the Bu₃SnH. Still other alternative conditions include maintaining a low concentration of tin hydride, which is more readily achieved by dropwise addition. Further, the concentration of catalyst and the temperature may also be manipulated to minimize the side reactions. In one illustrative embodiment, the hydrostannations are preformed with low concentrations of the alkynes 30 or 34 by a very slow dropwise addition of tributyltin hydride with low load of catalyst at 0° C. to afford the regio- and stereodefined vinylstannanes 31 or 35.

Stille coupling involves the palladium-catalyzed cross-coupling reaction between aryl or vinyl halides and triflates with organostannanes. See generally, Stille, J. K. The Palladium-Catalyzed Cross-Coupling Reactions of Organotin Reagents with Organic Electrophiles Angew. Chem. Int. Ed. 1986, 25, 508-523; Farina, V.; Krishnamurthy, V.; Scoot, W. J. Org. React. 1997, 50, 1-652; Littke, A. F.; Fu, G. C. Palladium-Catalyzed Coupling Reactions of Aryl Chlorides Angew. Chem. Int. Ed. 2002, 41, 4176-4211; Espinet, P.; Echavarren, A. M. The Mechanisms of the Stille Reaction Angew. Chem. Int. Ed. 2004, 43, 4704-4734, the disclosures of which are incorporated herein by reference. Alternative catalysts useful herein include Pd₂(dba)₃ and Pd(PPh₃)₄, solvents useful herein include toluene, dioxane, THF, and 1-methyl-2-pyrrolidinone (NMP) at 80° C., additives useful herein include CsF, PBu^{t}₃, AsPh₃, and PPh₃, and temperatures may range from room temperature to reflux temperature. However, it is appreciated that the transmetallation may be the rate-determining step of the Stille reaction. It has been shown that under certain conditions, copper iodide reacts with organostannanes to produce transient organocopper intermediates that are presumably more reactive than organostannanes towards transmetallation to palladium. See, Farina, V.; Kapadia, S.; Krishnan, B.; Wang, C.; Liebeskind, L. S. On the Nature of the “Copper Effect” in the Stille Cross-Coupling J. Org. Chem. 1994, 59, 5905-5911, the disclosure of which is incorporated herein by reference. Still other alternative conditions include vinylstannanes reacting with aryl iodides in the presence of CuI with CsF and AsPh₃ in THF at reflux. Still other alternative reaction conditions include Suzuki coupling of the vinyl iodide 32 with 3,4-dimethoxyphenylboronic acid in the presence of palladium acetate and 2-(di-t-butylphosphine)biphenyl proceeding to compound 3 in 62% yield. See, a) Miyaura, N.; Suzuki, A. Palladium-Catalyzed Cross-Coupling Reactions of Organoboron Compounds Chem. Rev. 1995, 95, 2457-2483, b) Suzuki, A. Recent Advances in the Cross-Coupling Reactions of Organoboron Derivatives with Organic Electrophiles, 1995-1998 J. Organomet. Chem. 1999, 576, 147-168, the disclosures of which are incorporated herein by reference.

Another embodiment of the processes described herein includes the coupling step illustrated by the production of compound 4 in 77% yield from the vinylstannane 35 and aryl iodide 29 in the presence of Pd(PBu^{t}₃)₂ with CsF in toluene at reflux temperature. These conditions are very general and practical and may be used in the processes described herein to synthesize the compounds described herein.

In another embodiment, the stereoselective syntheses of compounds of formula II via trifluoromethyl compounds 38 or 39, as illustrated by alkenyldiarylmethanes 7, 8 and 9, are outlined in Scheme 2. The synthesis also uses the Stille coupling of 1-bromo-3-fluoro-5-trifluoromethylbenzene (38) or 2-bromo-4-fluoro-1-trifluoromethylbenzene (39) with the tributyltin derivatives 31 or 35 in the presence of Pd(PBu^{t}₃)₂ with CsF in toluene at reflux temperature.

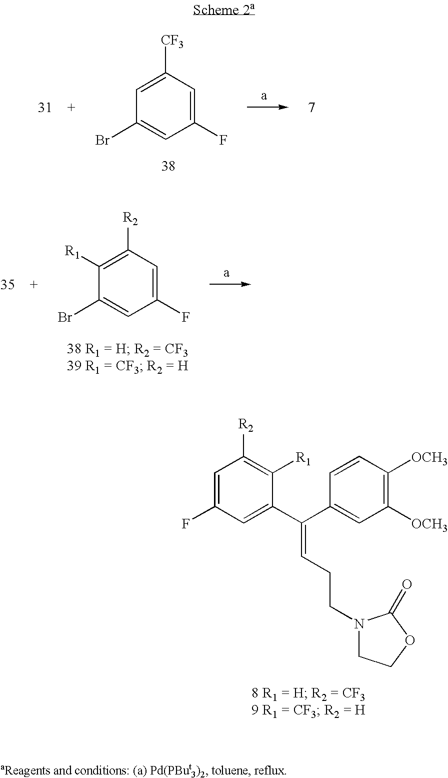

In another embodiment, vinylstannanes 43, 44, 51, and 52 are prepared by the processes described herein. As outlined in Scheme 3, the methylation of the phenol group of 45 using dimethyl sulfate in the presence of sodium hydroxide and tetrabutylammonium bromide as a phase-transfer catalyst in a mixture of dichloromethane and water at room temperature gave 2-bromo-4-chloro-1-methoxybenzene (47). Methyl 5-bromo-2-methoxy-3-methylbenzoate (48) was prepared by O-alkylation of 5-bromo-3-methylsalicylic acid (46) with dimethyl sulfate in acetone at reflux temperature, utilizing potassium carbonate as the base. The Sonogashira coupling of methyl 5-hexynoate with substituted aromatic bromides 38, 40, 47, and 48, followed by hydrostannation with tributyltin hydride, gave vinyl stannanes 43, 44, 51, and 52.

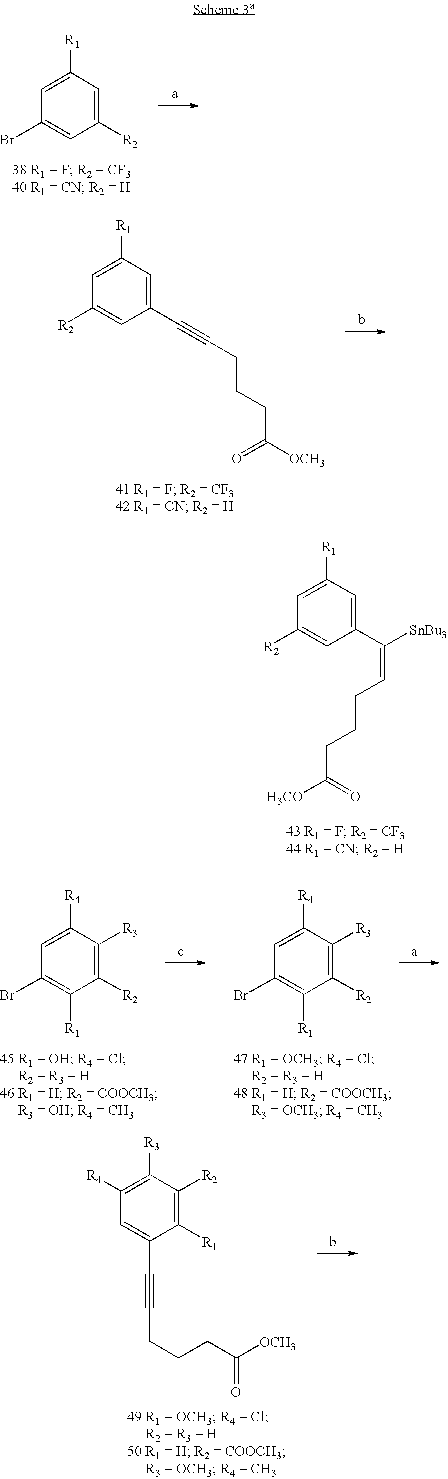

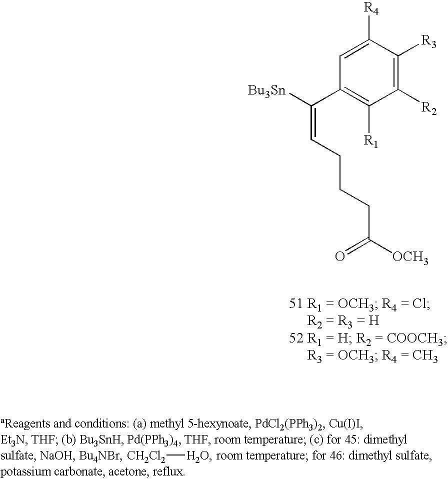

In another embodiment, outlined in Scheme 4, methylation of both the phenol and carboxylic acid groups of 53 using dimethyl sulfate in the presence of sodium hydroxide and tetrabutylammonium bromide as the phase-transfer catalyst in a mixture of dichloromethane and water at room temperature gave methyl 5-iodo-2-methoxybenzoate 54. In one aspect, chlorination of the aromatic ring at the C3 position of compound 54 is accomplished with SO₂Cl₂ in dichloromethane. Alternatively, the step is without solvent at 50° C. to afford the ester 55. The Stille coupling of vinyl stannanes 43, 44, 51, and 52 with different halo aromatic derivatives in the presence of Pd(PBu^{t}₃)₂ with CsF in toluene at reflux temperature gave alkenyldiarylmethanes 10-18.

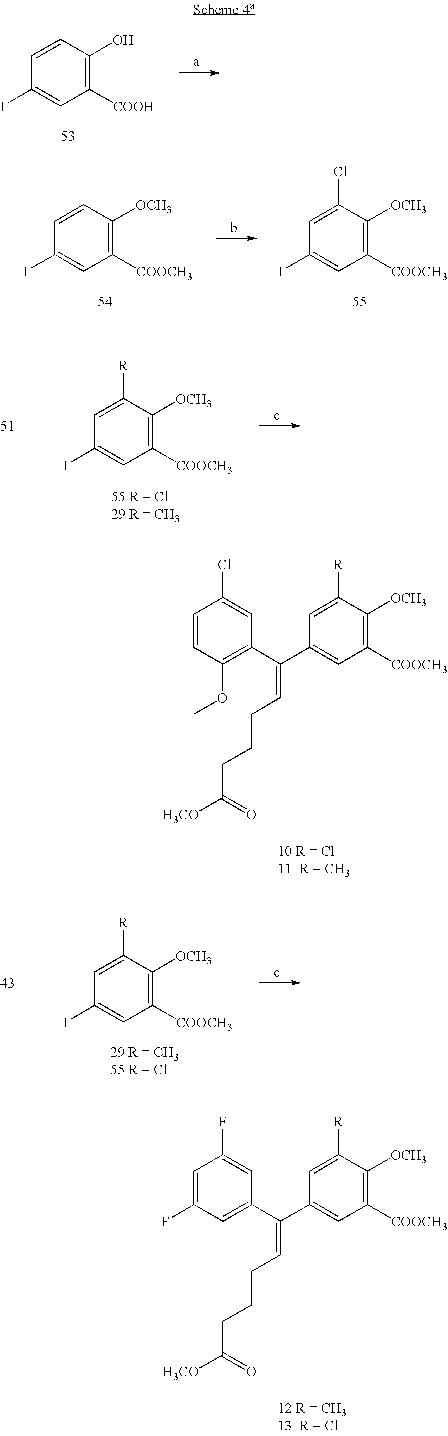

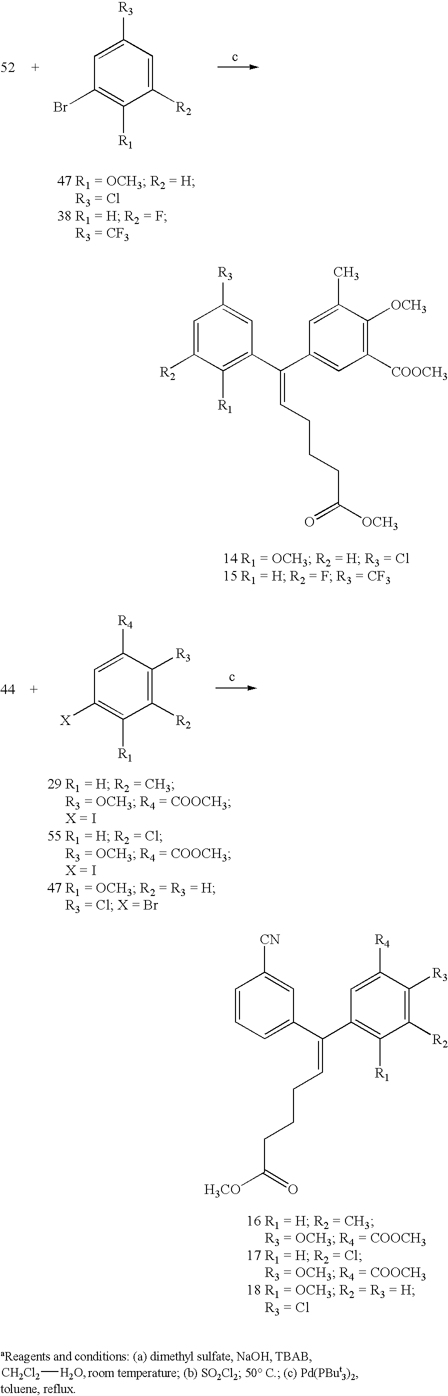

In another embodiment, it is appreciated the nitrogen of the isoxazole may mimic the carbonyl oxygen of the ester, and the methoxy group on the isoxazole may mimic the methoxy group on the methyl ester. It is understood that these compounds may be viewed as conformationally constrained ester mimics that may provide information about the biologically active conformation of the corresponding methyl ester in other compounds described herein. As shown in Scheme 5, 2,N-dihydroxy-5-iodo-3-methylbenzamide (56) was prepared from methyl 2-hydroxy-5-iodo-3-methylbenzoate (28) and hydroxylamine hydrochloride. 5-Iodo-7-methylbenzo[d]isoxazol-3-one (57) was synthesized from 2,N-dihydroxy-5-iodo-3-methylbenzamide (56) and carbonyldiimidazole. See, Friary, R.; Sunday B. R. A Direct Preparation of 3-Hydroxy-1,2-benzisoxazoles J. Heterocyclic Chem. 1979, 16, 1277, the disclosure of which is incorporated herein by reference. Methylation of 5-iodo-7-methylbenzo[d]isoxazol-3-one (57) with iodomethane afforded 5-iodo-2,7-dimethyl-benzo[d]isoxazol-3-one (58) and 5-iodo-3-methoxy-7-methylbenzo[d]isoxazole (59). The structure of 5-iodo-2,7-dimethyl-benzo[d]isoxazol-3-one (58) was confirmed by X-ray crystallography.

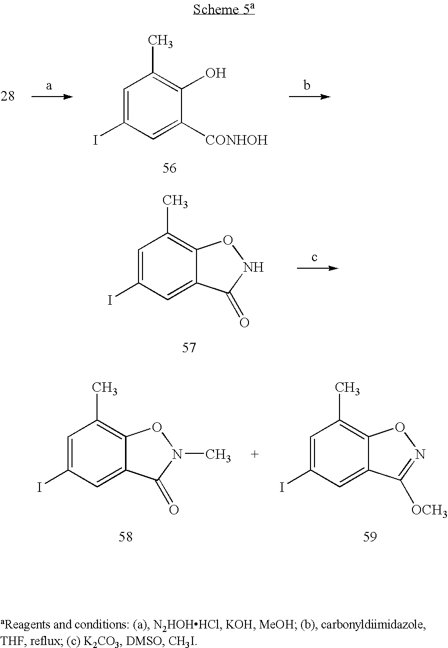

In another embodiment, the Stille approach was employed for the synthesis of compounds 19-22 having isoxazole as shown in Scheme 6. The Stille coupling of vinyl stannanes 43, 44, and 51 with different halo aromatic derivatives in the presence of Pd(PBu^{t}₃)₂ with CsF in toluene at reflux temperature gave compounds 19-22. Compound 22 was recrystallized from a mixture of ethyl acetate and hexanes to yield colorless needles, and the structure of compound 22 was confirmed by X-ray crystallography. Since the Stille coupling leads to the retention of the stereochemical integrity of the coupling partners, the E stereochemistry of the alkene 43 confirmed the regioselective cis addition during the hydrostannation reaction of the alkyne 41.

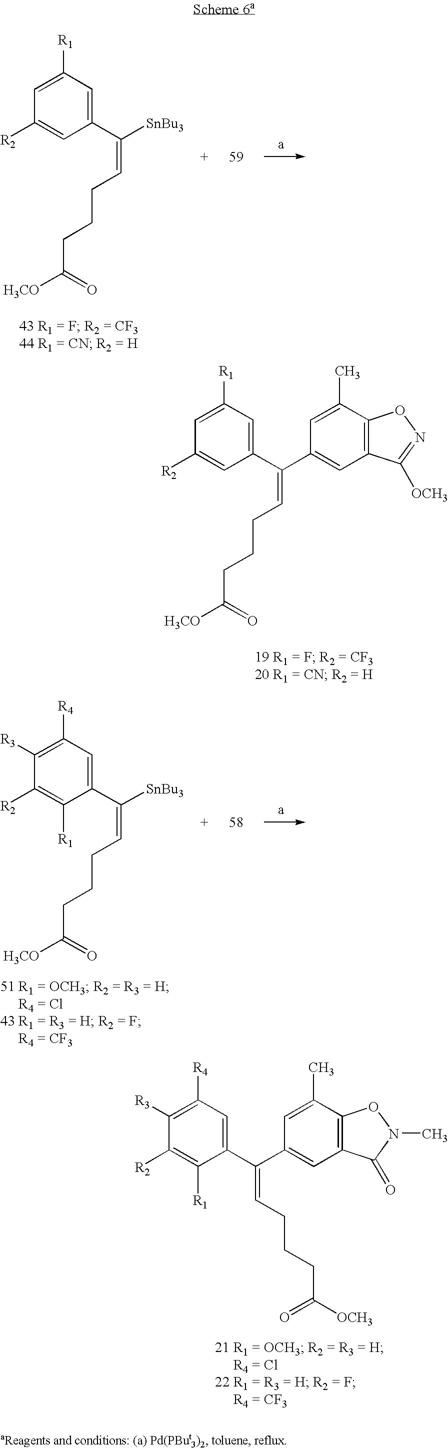

In another embodiment, a general process for preparing the compounds described herein includes the steps leading illustratively to intermediate 29, and subsequent use of metal-catalyzed reactions (Sonogashira reaction, hydrostannation, Stille coupling and Suzuki coupling). This process may be performed on large scale. In addition, it is appreciated that this process may be used for hydrostannation of sterically hindered internal alkynes and may be an improvement over conventional processes.

In another embodiment, compounds 60-68 are described, which include 4-methoxy-3-methoxycarbonyl-5-methylphenyl, 5-chloro-4-methoxy-3-methoxycarbonylphenyl, 4-methoxy-3-methoxycarbonylphenyl, and/or 3-fluoro-5-trifluoromethylphenyl groups. These compounds were prepared by the processes described herein comprising the steps of Sonogoshira and Stille cross-coupling reactions.

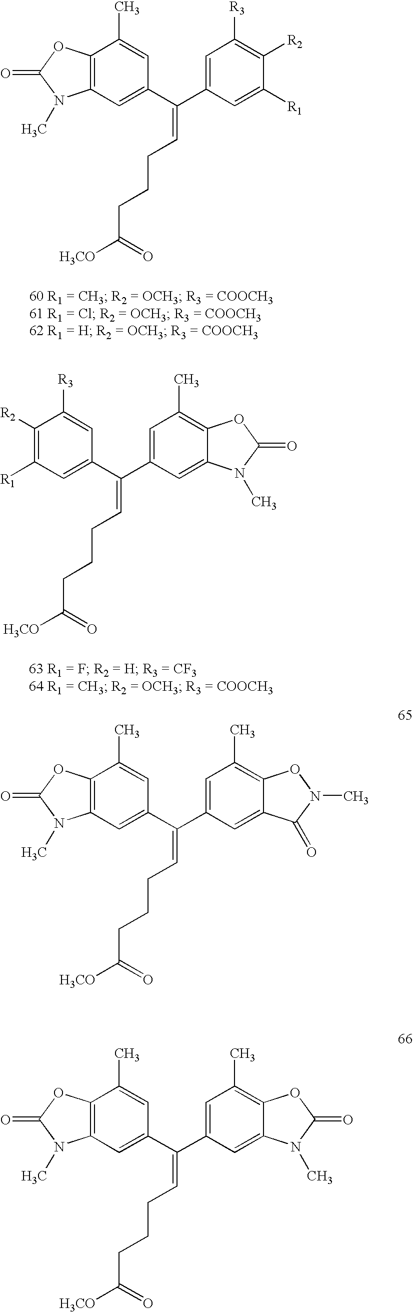

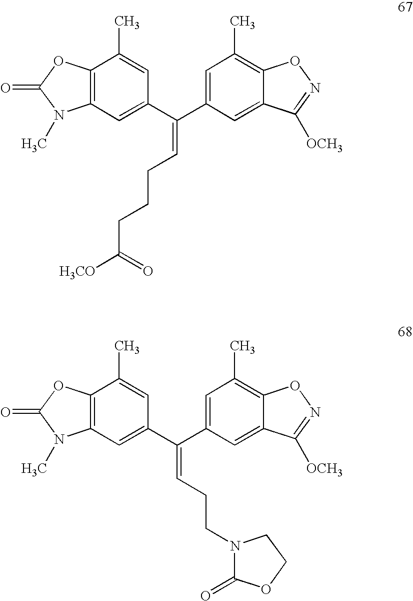

It is appreciated that the oxazole and isoxazole rings of compounds 60-68 may be more metabolically stable than other substituents. In particular, such rings may be more metabolically stable than the 4-methoxy-3-methoxycarbonylphenyl substituents found in other compounds of formulae IV described herein.

In another embodiment, processes for preparing compounds of formula IV are described. In one aspect, compounds of formula IV may be prepared by the general synthesis shown in Scheme 7, and illustrated for the preparation of compounds 65-67.

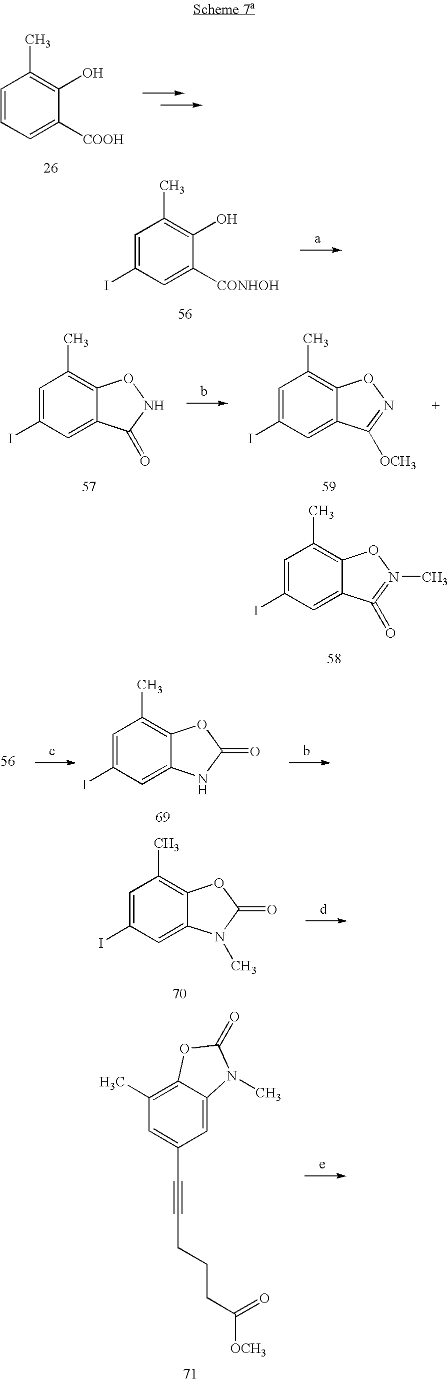

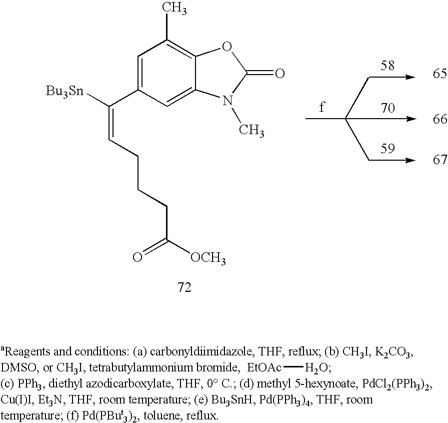

As outlined in Scheme 7, 5-diiodo-3-methoxy-7-methylbenzo[d]isoxazole (59) and 5-iodo-2,7-dimethyl-benzo[d]isoxazol-3-one (58) were synthesized in multi-step fashion from 3-methyl salicyclic acid (26) via 2,N-dihydroxy-5-iodo-3-methylbenzamide (56) and 5-iodo-7-methylbenzo[d]isoxazol-3-one (57). Benzoxazolones can be prepared by cyclization of 2-aminophenol derivatives by reaction with a source of a carbonyl group such as urea, N,N′-carbonyldiimidazole, triphosgene or ethyl chloroformate in different reaction conditions. See, Close, W. J.; Tiffany, B. D.; Spielman, M. A. The Analgestic Activity of Some Benzoxazolone Derivatives. J. Am. Chem. Soc. 1949, 71, 1265-1268; Nerenberg, J. B.; Erb, J. M.; Thompson, W. J.; Lee, H.-Y.; Guare, J. P.; Munson, P. M.; Bergman, J. M.; Huff, J. R.; Broten, T. P.; Chang, R. S. L.; Chen, T. B.; O'Malley, S.; Schorn, T. W.; Scott, A. L. Design and Synthesis of N-Alkylated Saccharins as Selective alpha-1A Adrenergic Receptor Antagonists. Bioorg. Med. Chem. Lett. 1998, 8, 2467-2472; Sicker, D. A Facile Synthesis of 6-Methoxy-2-oxo-2,3-dihydrobenzoxazole. Synthesis 1989, 875-876; Kluge, M.; Sicker, D. Synthesis of 4-Acetylbenzoxazoline-2(3H)-one Reported from Zea mays. J. Nat. Prod. 1998, 61, 821-822; Ünlü, S.; Baytas, S. N.; Kupeli, E.; Yesilada, E. Studies on Novel 7-Acyl-5-chloro-2-oxo-3H-benzoxazole Derivatives as Potential Analgesic and Anti-Inflammatory Agents. Arch. Pharm. Pharm. Med. Chem. 2003, 336, 310-321, the disclosures of which are incorporated herein by reference.

Alternatively, Moriarty et al. reported the synthesis of benzoxazolones by oxidation of salicylamides with iodobenzene diacetate in methanolic potassium hydroxide. See, Prakash, O.; Batra, H.; Kaur, H.; Sharma, P. K.; Sharma, V.; Singh, S. P.; Moriarty, R. M. Hypervalent Iodine Oxidative Rearrangement of Anthranilamides, Salicylamides and Some 13-Substituted Amides: A New and Convenient Synthesis of 2-Benzimidazolones, 2-Benzoxazolones and Related Compounds. Synthesis 2001, 541-543, the disclosure of which is incorporated herein by reference. With 2,N-dihydroxybenzamide 56 in hand, 5-iodo-7-methyl-3H-benzoxazol-2-one (69) was prepared from the 2,N-dihydroxybenzamide 56 under the Mitsunobu conditions (Ph₃P-DEAD) as shown in Scheme 2. Methylation of compound 69 with methyl iodide in the presence of potassium carbonate yielded compound 70, whose structure was confirmed by X-ray crystallography, establishing that the rearrangement in the Mitsunobu reaction occurred and methylation of compound 69 took place on nitrogen and not on oxygen. The Sonagashira coupling of methyl 5-hexynoate with compound 70 resulted in intermediate 71. Vinyl stannane 72 was synthesized by hydrostannation of intermediate 71 with tributyltin hydride in the presence of tetrakis(triphenylphosphine)palladium(0). The Stille coupling of vinyl stannane 72 with different halo aromatic derivatives 58, 70, 59 in the presence of Pd(PBu^{t}₂) with CsF in toluene at reflux temperature afforded alkenyldiarylmethanes 65-67.

In another embodiment, the Stille approach was also employed for the synthesis of compounds 60-64, which have nonidentical aromatic rings, and compound 1, having identically substituted aromatic rings, as shown in Scheme 8.

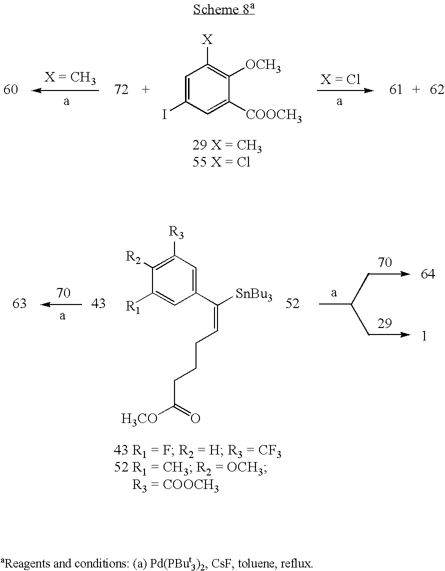

As outlined in Scheme 8, 3-Chloro-5-iodo-2-methoxybenzoic acid methyl ester (55) was synthesized as shown in Scheme 4 above, and the vinyl stannanes 43 and 52 were synthesized as shown in Scheme 3 above. The Stille coupling of vinyl stannanes 72, 43 and 52 with different halo aromatic derivatives 29 and 70 afforded alkenyldiarylmethanes 60, 63 and 64 in the presence of Pd(PBu^{t}₃)₂ with CsF in toluene at reflux temperature. The attempted Stille cross-coupling of vinylstannane 72 with the aryl iodide 55 under standard conditions led to the generation of the desired coupling product 61 along with dehalogenated product 62. The dehalogenation of aryl halides commonly occurs during metal-catalyzed cross-coupling reactions. See, Peters, D.; Hornfeldt, A. B.; Gronowitz, S. Synthesis of 5-Cyclopropylurcil and 5-Cyclopropylcytosine by the Pd(0)-Catalyzed Coupling Reaction. J. Heterocycl. Chem. 1991, 28, 1629-1631; Navarro, O.; Kaur, H.; Mahjoor, P.; Nolan, S. P. Cross-Coupling and Dehalogenation Reactions Catalyzed by (N-Heterocyclic Carbene)Pd(allyl)Cl Complexes. J. Org. Chem. 2004, 69, 3173-3180; Handy, S. T.; Bregman, H.; Lewis, J.; Zhang, X.; Y., Z. An Unusual Dehalogenation in the Suzuki Coupling of 4-Bromopyrrole-2-carboxylates. Tetrahedron Lett. 2003, 44, 427-430, the disclosures of which are incorporated herein by reference. Compound 1 may be synthesized by McMurry reaction of methyl 5-oxopentanoate with a symmetrical benzophenone, di(4-methoxy-3-methoxycarbonyl-5-methylphenyl) ketone, in the presence of low-valent titanium species in 49% yield. See, Xu, G.; Micklatcher, M.; Silvestri, M.; Hartman, T. L.; Burrier, J.; Osterling, M. C.; Wargo, H.; Turpin, J. A.; Buckheit, R. W., Jr.; Cushman, M. The Biological Effects of Structural Variation at the Meta Position of the Aromatic Rings and at the End of the Alkenyl Chain in the Alkenyldiarylmethane Series of Non-Nucleoside Reverse Transcriptase Inhibitors. J. Med. Chem. 2001, 44, 4092-4113, the disclosure of which is incorporated herein by reference. Compound 1 was also prepared using the Stille reaction in 68% yield from the vinylstannane 52 and aryl iodide 29.

In another embodiment, the Stille approach was also employed for the synthesis of compound 68, which has nonidentical aromatic rings, as shown in Scheme 9.

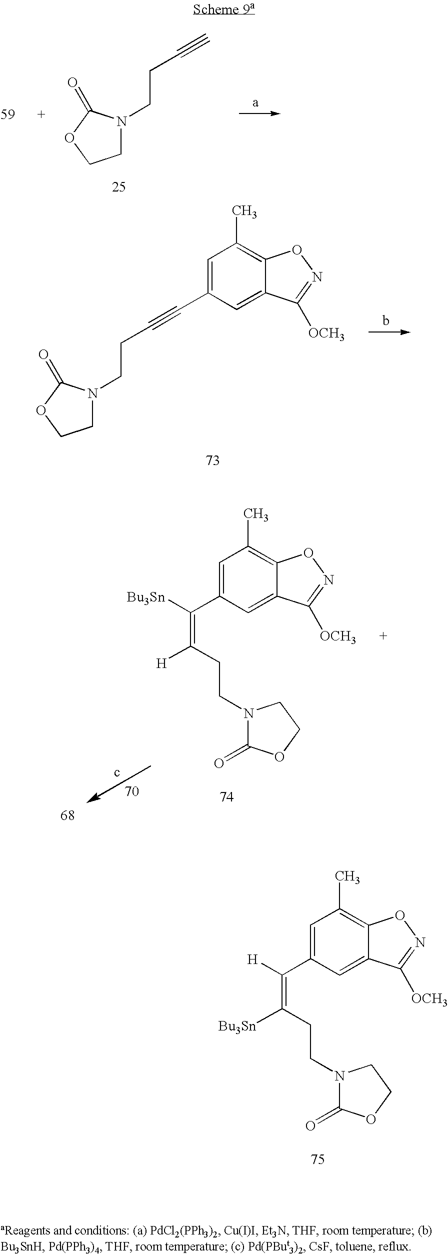

As outlined in Scheme 9, the Sonogashira coupling of compound 59 with 3-but-3-ynyl-1,3-oxazolidin-2-one (25) yielded the disubstituted alkyne 73 in a manner analogous to Schemes 7 and 8 above. The hydrostannation of 73 with tri-n-butyltin hydride in the presence of Pd(PPh₃)₄ afforded the regiochemically and stereochemically defined vinylstannane 74 and the side product 75, which were separated chromatographically. Compound 68 was then synthesized from the vinylstannane 74 and the aryl iodide 70 in the presence of Pd(PBu^{t}₃)₂ with CsF in toluene at reflux temperature.

In another embodiment, stabilized compounds of formulae I-IV are described herein. Improved stability may be evaluated by measuring the half-life of the compounds in rat blood plasma.

### EXAMPLES

NMR spectra were obtained at 300 MHz (¹H) and 75 MHz (¹³C) in CDCl₃ using CHCl₃ as internal standard. Flash chromatography was performed with 230-400 mesh silica gel. TLC was carried out using Baker-flex silica gel IB2-F plates of 2.5 mm thickness. Melting points are uncorrected. Unless otherwise stated, chemicals and solvents were of reagent grade and used as obtained from commercial sources without further purification. Tetrahydrofuran (THF) was freshly distilled from sodium/benzophenone ketyl radical prior to use. Dichloromethane was freshly distilled from calcium hydride prior to use. Lyophilized rat plasma (lot 052K7609) was obtained from Sigma Chemical Co., St. Louis, Mo. All yields given refer to isolated yields.

Methyl 4′,4″-Dimethoxy-3′,3″-di(methoxycarbonyl)-5′,5″-dimethyl-6,6-diphenyl-5-hexenoate (1). The general procedure was followed using vinylstannane 52 (246 mg, 0.413 mmol), aryl iodide 29 (167 mg, 0.547 mmol), cesium fluoride (259 mg, 1.69 mmol) and Pd(PBu^{t}₃)₂ (24 mg, 0.045 mmol) in toluene (1 mL). The mixture was stirred under argon at room temperature for 15 h, at 60° C. for 7 h and at 110° C. for 17 h. The residue was purified by column chromatography on silica gel (20 g), eluting with EtOAc-hexanes (0-30%) to afford the product 1 (136 mg) as an oil in 68% yield. ¹H NMR¹⁰ δ 7.44 (d, J=2.4 Hz, 1H), 7.38 (d, J=2.1 Hz, 1H), 7.07 (m, 2H), 5.94 (t, J=7.5 Hz, 1H), 3.88 (s, 3H), 3.87 (s, 3H), 3.85 (s, 3H), 3.79 (s, 3H), 3.61 (s, 3H), 2.30 (s, 3H), 2.28 (m, 2H), 2.23 (s, 3H), 2.09 (m, 2H), 1.74 (m, 2H).

(E)-6-[1-3,4-Dimethoxyphenyl]-4-(2-oxo-oxazolidin-3-yl)-but-1-enyl]-2-methoxy-3-methyl-benzoic Acid Methyl Ester (3). Method I: A mixture of the vinylstannane 31 (100 mg, 0.164 mmol), aryl iodide 33 (44.7 mg, 0.169 mmol), triphenylarsine (21.3 mg, 0.068 mmol), copper(I) iodide (10.0 mg, 0.053 mmol) and tris(dibenzylideneacetone)dipalladium (15 mg, 0.016 mmol) in 1-methyl-2-pyrrolidinone (NMP) (4 mL) was heated at 80° C. under argon atmosphere for 14 h, cooled to room temperature, filtered through a pad of Celite, and washed with ethyl acetate and dichloromethane. The filtrate was concentrated to afford a residue. The residue was purified by column chromatography on silica gel (50 g) using ethyl acetate in hexanes (0-30%) to afford the product 3 (32 mg) in 43% yield.

Method II: A mixture of the vinyl iodide 32 (353 mg, 0.793 mmol), palladium acetate (9.0 mg, 0.039 mmol), 2-(di-t-butylphosphine)biphenyl (24.2 mg, 0.080 mmol), potassium fluoride (147 mg, 2.48 mmol) and 3,4-dimethoxyphenylboronic acid (321 mg, 1.764 mmol) in THF (4.0 mL) was stirred at room temperature for 14 h and at 60° C. for 8.5 h. The reaction mixture was cooled to room temperature. Ethyl ether (30 mL) was added to dilute the mixture. The mixture was washed with aqueous 10% potassium hydroxide (2×20 mL). The organic phase was collected and washed with brine (30 mL). The aqueous solution was extracted with ethyl acetate (3×15 mL), washed with brine (20 mL), and then combined with the ether solution, dried over anhydrous sodium sulfate and concentrated. The residue was purified by column chromatography on silica gel (40 g) using ethyl acetate in hexanes (0-50%) to afford the product 3 (0.224 g) as an oil in 62% yield. IR (KBr film): 2926, 2851, 1751, 1729, 1599, 1580, 1513, 1481, 1438, 1378, 1262, 1142, 1025, 804, 762 cm⁻¹; ¹H NMR (300 MHz, CDCl₃) δ 7.42 (d, J=2.4 Hz, 1H), 7.12 (d, J=1.5 Hz, 1H), 6.80 (d, J=2.1 Hz, 1H), 6.74 (d, J=8.4 Hz, 1H), 6.63 (dd, J=2.1 Hz, J=8.4 Hz, 1H), 5.92 (t, J=7.5 Hz, 1H), 4.23 (t, J=8.1 Hz, 2H), 3.88 (s, 3H), 3.86 (s, 3H), 3.37 (t, J=6.9 Hz, 2H), 3.32 (t, J=8.1 Hz, 2H), 2.37 (m, 2H), 2.30 (s, 3H); ¹³C NMR (75 MHz, CDCl₃) δ 166.8, 158.5, 157.5, 148.6, 142.7, 136.3, 135.1, 135.0, 132.8, 130.2, 124.4, 120.3, 110.7, 110.5, 61.6, 56.0, 55.9, 52.2, 44.2, 44.0, 27.9, 16.1; ESIMS m/z (rel intensity) 478 (MNa⁺, 100). Anal. (C₂₅H₂₉NO₇) C, H, N.

General Procedure for Synthesis of Alkenyldiarylmethanes by the Cross-Coupling Reaction of Vinylstannanes with Aromatic Iodides or Bromides. A mixture of vinylstannane (1 equiv), iodide or bromide (1.2-1.5 equiv), cesium fluoride (3.0-4.5 equiv), and Pd(PBu^{t}₃)₂ (10 mol %) in toluene (1 mL) under argon was stirred at room temperature for 7.3-24 h and at 90-110° C. for 9.5-43 h. The reaction mixture was cooled to room temperature, filtered through a short column of silica gel (5 g), and the column washed with ethyl acetate. The organic solution was concentrated.

(Z)-5-[1-(3,4-Dimethoxyphenyl)-4-(2-oxo-oxazolidin-3-yl)-but-1-enyl]-2-methoxy-3-methyl-benzoic Acid Methyl Ester (4). The general procedure was followed using the vinylstannane derivative 35 (367.7 mg, 0.649 mmol), iodide 29 (209.6 mg, 0.685 mmol), cesium fluoride (306 mg, 2.0 mmol), and Pd(PBu^{t}₃)₂ (53 mg, 0.102 mmol) in toluene (3 mL). After the mixture was stirred at room temperature for 22 h, and at 80° C. for 5.5 h, compound 29 (103.5 mg, 0.34 mmol) and Pd(PBu^{t}₃)₂ (9.7 mg, 0.019 mmol) were added. The mixture was heated 80° C. for another 16 h. The residue was purified by column chromatography on silica gel (40 g) using ethyl acetate in hexanes (0-50%) to afford the product 4 (227 mg) as an oil in 77% yield. IR (KBr) 2932, 1751, 1579, 1580, 1513, 1481, 1437, 1320, 1257, 1138, 1027, 804, 762 cm⁻¹; ¹H NMR (300 MHz, CDCl₃) δ 7.43 (d, J=2.4 Hz, 1H), 7.17 (d, J=2.1 Hz, 1H), 6.87 (d, J=8.4 Hz, 1H), 6.71 (dd, J=2.1 Hz, J=8.1 Hz, 1H), 6.61 (d, J=1.8 Hz), 5.92 (t, J=7.5 Hz, 1H), 4.22 (t, J=8.1 Hz, 2H), 3.90 (s, 3H), 3.86 (s, 3H), 3.82 (s, 3H), 3.79 (s, 3H), 3.36 (t, J=6.9 Hz, 2H), 3.32 (t, J=7.2 Hz, 2H), 2.40 (m, 2H), 2.24 (s, 3H); ¹³C NMR (75 MHz, CDCl₃) δ 166.7, 158.1, 157.2, 148.5, 148.0, 142.5, 137.6, 133.6, 132.1, 131.5, 127.4, 124.9, 124.0, 121.8, 112.5, 110.7, 61.4, 61.2, 55.7, 55.6, 51.9, 44.0, 43.7, 27.6, 15.9; ESIMS m/z (rel intensity) 478 (MNa⁺, 100). Anal. (C₂₅H₂₉NO₇) C, H, N.

(E)-5-[1-(3-Fluoro-5-trifluoromethylphenyl)-4-(2-oxo-oxazolidin-3-yl)-but-1-enyl]-2-methoxy-3-methylbenzoic Acid Methyl Ester (7). The general procedure was followed using the vinylstannane 31 (323 mg, 0.531 mmol), bromide 38 (219 mg, 0.874 mmol), cesium fluoride (280 mg, 1.825 mmol) and Pd(PBu^{t}₃)₂ (28.4 mg, 0.054 mmol) in toluene (1 mL). The mixture was stirred at room temperature for 24 h, at 60° C. for 25 h and at 110° C. for 22 h. The residue was purified by column chromatography on silica gel (30 g), eluting with EtOAc-hexanes (0-50%) to afford the product 7 (82.5 mg) as an oil in 32% yield. IR (KBr) 2952, 1752, 1600, 1482, 1438, 1351, 1264, 1236, 1205, 1171, 1128, 1094, 1034, 1008, 939, 875, 801, 762, 700 cm⁻¹; ¹H NMR (300 MHz, CDCl₃) δ 7.40 (d, J=2.4 Hz, 1H), 7.17 (d, J=8.1 Hz, 1H), 7.09 (d, J=1.8 Hz, 1H), 7.03 (d, J=9.6 Hz, 1H), 6.08 (t, J=7.5 Hz, 1H), 4.24 (t, J=7.8 Hz, 2H), 3.89 (s, 3H), 3.84 (s, 3H), 3.40-3.31 (m, 4H), 2.38 (m, 2H), 2.31 (s, 3H); ¹³C NMR (75 MHz, CDCl₃) δ 166.5, 158.4, 157.9, 145.2, 140.9, 136.0, 133.5, 130.1, 128.8, 124.8, 119.7, 118.0, 111.9, 61.6, 52.4, 44.4, 43.8, 28.1, 16.2; ESIMS m/z (rel intensity) 504.09 (MNa⁺, 50). Anal. (C₂₄H₂₃F₄NO₅) C, H, F, N.

(E)-3-[4-(3-Fluoro-5-trifluoromethylphenyl)-4-(3,4-dimethoxyphenyl)-but-3-enyl]-oxazolidin-2-one (8). The general procedure was followed using the vinylstannane 35 (178.4 mg, 0.315 mmol), bromide 38 (130 mg, 0.519 mmol), cesium fluoride (179 mg, 1.17 mmol) and Pd(PBu^{t}₃)₂ (17.8 mg, 0.034 mmol) in toluene (1 mL). After the mixture was stirred at room temperature for 18 h and at 90° C. for 4 h. More bromide 38 (147.6 mg, 0.589 mmol) and Pd(PBu^{t}₃)₂ (8.8 mg, 0.017 mmol) were added. The mixture was heated at 90° C. for 22.5 h and at 115° C. for 26 h. The residue was purified by column chromatography on silica gel (25 g) using ethyl acetate in hexanes (0-50%) to afford the product 8 (45.2 mg) as an oil in 33% yield. IR (KBr) 2917, 2849, 1751, 1600, 1582, 1514, 1484, 1466, 1441, 1350, 1255, 1232, 1200, 1169, 1130, 1093, 1027, 971, 927, 868, 814, 763, 700 cm⁻¹; ¹H NMR (300 MHz, CDCl₃) δ 7.25 (s, 1H), 7.17 (d, J=8.1 Hz, 1H), 7.10 (d, J=9.9 Hz, 1H), 6.89 (d, J=8.4 Hz, 1H), 6.71 (dd, J=2.1 Hz, J=8.1 Hz, 1H), 6.60 (d, J=1.8 Hz, 1H), 6.064 (t, J=7.5 Hz, 1H), 4.22 (t, J=7.8 Hz, 2H), 3.91 (s, 3H), 3.82 (s, 3H), 3.38 (t, J=6.9 Hz, 2H), 3.32 (t, J=7.8 Hz, 2H), 2.43 (q, J=7.2 Hz, J=6.9 Hz, 2H); ¹³C NMR (75 MHz, CDCl₃) δ 164.0, 160.7, 158.3, 149.0, 148.6, 145.8, 141.9, 130.6, 127.7, 122.1, 119.7, 117.8, 117.5, 112.5, 111.4, 111.1, 61.5, 55.9, 55.8, 44.2, 43.8, 27.9; ESIMS m/z (rel intensity) 440 (MH⁺, 48). Anal. (C₂₂H₂₁F₄NO₄) C, H, F, N.

(E)-3-[4-(5-Fluoro-2-trifluoromethylphenyl)-4-(3,4-dimethoxyphenyl)-but-3-enyl]-oxazolidin-2-one (9). The general procedure was followed using the vinylstannane 35 (84 mg, 0.148 mmol), bromide 39 (0.032 mL, 0.224 mmol), cesium fluoride (84 mg, 0.55 mmol), and Pd(PBu^{t}₃)₂ (8.9 mg, 0.017 mmol) in toluene (1 mL). The mixture was stirred at room temperature for 19 h and at 90° C. for 22 h. The residue was purified by column chromatography on silica gel (25 g) using ethyl acetate in hexanes (0-50%) to afford the product 9 (14.7 mg) as an oil in 23% yield and the starting materials 35 (16.3 mg) in 19.4% yield. IR (KBr) 2929, 1751, 1611, 1583, 1514, 1428, 1364, 1310, 1260, 1234, 1160, 1137, 1104, 1047, 1026, 826, 763 cm⁻¹; ¹H NMR (300 MHz, CDCl₃) δ 7.62 (dd, J=5.4 Hz, J=8.7 Hz, 1H), 7.06-6.96 intensity) 440 (MH⁺, 100). Anal. (C₂₂H₂₁F₄NO₄) C, H, N.

(Z)-3-Chloro-5-[1-(5-chloro-2-methoxyphenyl)-5-methoxycarbonyl-pent-1-enyl]-2-methoxybenzoic Acid Methyl Ester (10). The general procedure was followed using the vinylstannane 51 (283.8 mg, 0.509 mmol), iodide 55 (254.4 mg, 0.779 mmol), cesium fluoride (355 mg, 2.314 mmol) and Pd(PBu^{t}₃)₂ (32.2 mg, 0.062 mmol) in toluene (1 mL). The mixture was stirred at room temperature for 7.3 h and at 110° C. for 24 h. The residue was purified by column chromatography on silica gel (25 g), eluting with EtOAc-hexanes (0-5%) to afford the product 10 (28 mg) as an oil in 12% yield. IR (KBr) 2950, 1735, 1487, 1436, 1248, 1208, 1130, 1000, 810 cm⁻¹; ¹H NMR (300 MHz, CDCl₃) δ 7.52 (d, J=2.4 Hz, 1H), 7.27 (dd, J=2.7 Hz, J=8.7 Hz, 1H), 7.25 (d, J=2.4 Hz, 1H), 6.99 (d, J=2.7 Hz, 1H), 6.85 (d, J=9.0 Hz, 1H), 6.10 (t, J=7.5 Hz, 1H), 3.88 (s, 6H), 3.67 (s, 3H), 3.60 (s, 3H), 2.25 (t, J=7.5 Hz, 2H), 2.02-1.95 (m, 2H), 1.77-1.70 (m, 2H); ¹³C NMR (75 MHz, CDCl₃) δ 173.8, 166.0, 155.6, 154.4, 138.0, 135.5, 131.6, 130.8, 129.2, 128.9, 127.1, 126.4, 125.5, 112.4, 61.9, 55.7, 52.4, 51.5, 33.3, 29.3, 24.3; ESIMS m/z (rel intensity) 466.78/468.83 (MH⁺, 85/51). Anal. (C₂₃H₂₄Cl₂O₆) C, H, Cl.

(Z)-5-[1-(5-Chloro-2-methoxyphenyl)-5-methoxycarbonyl-pent-1-enyl]-2-methoxy-3-methylbenzoic Acid Methyl Ester (11). The general procedure was followed using the vinylstannane 51 (260 mg, 0.466 mmol), iodide 29 (219 mg, 0.715 mmol), cesium fluoride (222 mg, 1.447 mmol) and Pd(PBu^{t}₃)₂ (21.7 mg, 0.042 mmol) in toluene (1 mL). The mixture was stirred at room temperature for 7.3 h and at 110° C. for 24 h. The residue was purified by column chromatography on silica gel (20 g), eluting with EtOAc-hexanes (0-5%) to afford the product 11 (29 mg) as an oil in 14% yield. IR (KBr) 2949, 1731, 1486, 1436, 1248, 1121, 1008, 884, 810 cm⁻¹; ¹H NMR (300 MHz, CDCl₃) δ 7.47 (d, J=2.4 Hz, 1H), 7.27 (dd, J=1.2 Hz, J=10.2 Hz, 1H), 7.09 (d, J=2.4 Hz, 1H), 7.00 (d, J=2.7 Hz, 1H), 6.86 (d, J=8.7 Hz, 1H), 6.08 (t, J=7.5 Hz, 1H), 3.87 (s, 3H), 3.78 (s, 3H), 3.68 (s, 3H), 3.61 (s, 3H), 2.27 (t, J=7.5 Hz, 2H), 2.23 (s, 3H), 2.02-1.94 (q, 2H), 1.78-1.68 (m, 2H); ¹³C NMR (75 MHz, CDCl₃) δ 173.9, 167.0, 157.3, 155.7, 136.6, 136.4, 132.8, 132.3, 130.8, 130.2, 129.8, 128.5, 126.7, 125.4, 124.2, 112.3, 61.5, 55.8, 52.2, 51.5, 33.4, 29.3, 24.5, 16.2; ESIMS m/z (rel intensity) 469.12 (MNa⁺, 100), 471.04 (MNa⁺, 34). Anal. (C₂₄H₂₇ClO₆) C, H, Cl.

(Z)-5-[1-(3-Fluoro-5-trifluoromethylphenyl)-5-methoxycarbonyl-pent-1-enyl]-2-methoxy-3-methylbenzoic Acid Methyl Ester (12). The general procedure was followed using the vinylstannane 43 (250 mg, 0.43 mmol), iodide 29 (201.8 mg, 0.66 mmol), cesium fluoride (230 mg, 1.5 mmol) and Pd(PBu^{t}₃)₂ (25 mg, 0.048 mmol) in toluene (1 mL). The mixture was stirred at room temperature for 7 h and at 110° C. for 24 h. The residue was purified by column chromatography on silica gel (20 g), eluting with EtOAc-hexanes (0-10%) to afford the product 12 (118 mg) as an oil in 58% yield. IR (KBr) 2953, 1734, 1599, 1481, 1437, 1375, 1320, 1264, 1234, 1209, 1170, 1130, 1090, 1008, 937, 882, 800, 770, 702 cm⁻¹; ¹H NMR (300 MHz, CDCl₃) δ 7.42 (d, J=2.4 Hz, 1H), 7.28 (m, 1H), 7.19 (s, 1H), 7.05-7.02 (m, 2H), 6.04 (t, J=7.5 Hz, 1H), 3.88 (s, 3H), 3.80 (s, 3H), 3.61 (s, 3H); 2.28 (t, J=7.5 Hz, 2H), 2.24 (s, 3H), 2.13-2.06 (q, 2H), 1.82-1.72 (m, 2H); ¹³C NMR (75 MHz, CDCl₃) δ 173.6, 166.8, 164.0, 160.7, 157.7, 142.8, 139.2, 136.6, 133.6, 132.8, 130.9, 127.5, 124.6, 122.3, 120.4, 120.1, 111.9, 111.6, 61.5, 52.2, 51.5, 33.3, 29.0, 24.8, 16.1; ESIMS m/z (rel intensity) 491.13 (MNa⁺, 100). Anal. (C₂₄H₂₄F₄O₅) C, H, F.

(E)-3-Chloro-5-[1-(3-fluoro-5-trifluoromethylphenyl)-5-methoxycarbonyl-pent-1-enyl]-2-methoxy-benzoic Acid Methyl Ester (13). The general procedure was followed using the vinylstannane 43 (337 mg, 0.58 mmol), iodide 55 (289 mg, 0.885 mmol), cesium fluoride (300 mg, 1.96 mmol) and Pd(PBu^{t}₃)₂ (31 mg, 0.059 mmol) in toluene (1 mL). The mixture was stirred at room temperature for 23 h, at 60° C. for 24 h and at 100° C. for 24 h. The residue was purified by column chromatography on silica gel (20 g), eluting with EtOAc-hexanes (0-5%) to afford the product 13 (13.2 mg) as an oil in 5% yield. IR (KBr) 2953, 1737, 1599, 1478, 1438, 1374, 1314, 1264, 1207, 1171, 1131, 1093, 1000, 929, 878, 797, 744, 702 cm⁻¹; ¹H NMR (300 MHz, CDCl₃) δ 7.46 (d, J=2.4 Hz, 1H), 7.30 (m, 2H), 7.26 (d, J=7.26, 1H), 7.18 (s, 1H), 6.09 (t, J=7.5 Hz, 1H), 3.91 (s, 3H), 3.87 (s, 3H), 3.60 (s, 3H), 2.28 (t, J=7.5 Hz, 2H), 2.14-2.07 (q, 2H), 1.82-1.72 (m, 2H); ESIMS m/z (rel intensity) 511.18 (MNa⁺, 100). Anal. (C₂₃H₂₁ClF₄O₅) C, H, Cl, F.

(E)-5-[1-(5-Chloro-2-methoxyphenyl)-5-methoxycarbonyl-pent-1-enyl]-2-methoxy-3-methylbenzoic Acid Methyl Ester (14). The general procedure was followed using the vinyl tributylstannane 52 (166 mg, 0.279 mmol), bromide 47 (111 mg, 0.50 mmol), cesium fluoride (122 mg, 0.787 mmol) and Pd(PBu^{t}₃)₂ (13.2 mg, 0.025 mmol) in toluene (1 mL). The mixture was stirred at room temperature for 2 h and at 110° C. for 27.5 h. The residue was purified by column chromatography on silica gel (25 g), eluting with EtOAc-hexanes (0-5%) to afford the product 14 (50.1 mg) as an oil in 40% yield. IR (KBr) 2950, 1732, 1591, 1484, 1436, 1366, 1291, 1252, 1231, 1195, 1170, 1124, 1010, 883, 808 cm⁻¹; ¹H NMR (300 MHz, CDCl₃) δ 7.39 (d, J=2.1 Hz, 1H), 7.16 (dd, J=2.7 Hz, J=8.7 Hz, 1H), 7.10 (d, J=2.7 Hz, 1H), 7.07 (d, J=2.1 Hz, 1H), 6.72 (d, J=8.7 Hz, 1H), 3.87 (s, 3H), 3.79 (s, 3H), 3.61 (s, 3H), 3.55 (s, 3H), 2.29 (m, 2H), 2.26 (s, 3H), 2.19 (m, 2H), 1.75 (m, 2H); ESIMS m/z (rel intensity) 469 (MNa⁺, 100), 471 (MNa⁺, 39). Anal. (C₂₄H₂₇ClO₆) C, H, Cl.

(E)-5-[1-(3-Fluoro-5-trifluoromethylphenyl)-5-methoxycarbonyl-pent-1-enyl]-2-methoxy-3-methylbenzoic Acid Methyl Ester (15). The general procedure was followed using the vinylstannane 52 (365 mg, 0.613 mmol), bromide 38 (268 mg, 1.07 mmol), cesium fluoride (358 mg, 2.33 mmol) and Pd(PBu^{t}₃)₂ (34.2 mg, 0.066 mmol) in toluene (1 mL). The mixture was stirred at room temperature for 5.5 h, at 60° C. for 16 h and at 100° C. for 26 h. The residue was purified by column chromatography on silica gel (25 g), eluting with EtOAc-hexanes (0-5%) to afford the product 15 (210.5 mg) as an oil in 73% yield. IR (KBr) 2952, 1734, 1600, 1437, 1350, 1257, 1201, 1169, 1128, 1094, 1009, 873 cm⁻¹; ¹H NMR (300 MHz, CDCl₃) δ 7.38 (d, J=2.1 Hz, 1H), 7.28 (s, 1H), 7.16 (d, J=8.1 Hz, 1H), 7.07 (d, J=2.1 Hz, 1H), 6.96 (d, J=9.9 Hz, 1H), 6.09 (d, J=7.5 Hz, 1H), 3.89 (s, 3H), 3.86 (s, 3H), 3.61 (s, 3H), 2.31 (s, 3H), 2.27 (m, 2H), 2.14 (m, 2H), 1.77 (m, 2H); ESIMS m/z (rel intensity) 491 (MNa⁺, 100). Anal. (C₂₄H₂₄F₄O₅) C, H, F.

(E)-5-[5-Carboxy-1-(3-cyanophenyl)-pent-1-enyl]-2-methoxy-3-methyl benzoic Acid Methyl Ester (16). The general procedure was followed using the vinylstannane 44 (212 mg, 0.409 mmol), iodide 29 (197.5 mg, 0.645 mmol), cesium fluoride (190 mg, 1.24 mmol) and Pd(PBu^{t}₃)₂ (23 mg, 0.044 mmol) in toluene (1 mL). The mixture was stirred at room temperature for 65 h, at 65° C. for 8.5 h and at 110° C. for 22 h. The residue was purified by column chromatography on silica gel (25 g), eluting with EtOAc-hexanes (0-10%) to afford the product 16 (98 mg) as an oil in 59% yield. IR (KBr) 2951, 2230, 1731, 1645, 1480, 1436, 1318, 1263, 1201, 1124, 1007, 770 cm⁻¹; ¹H NMR (300 MHz, CDCl₃) δ 7.58 (d, J=7.8 Hz, 1H), 7.46 (t, J=7.8 Hz, 1H), 7.40-7.34 (m, 3H), 7.03 (d, J=2.4 Hz, 1H), 6.02 (t, J=7.5 Hz, 1H), 3.84 (s, 3H), 3.77 (s, 3H), 3.58 (s, 3H), 2.25 (t, J=7.5 Hz, 2H), 2.21 (s, 3H), 2.06 (q, J=7.5 Hz, 2H), 1.78-1.69 (m, 2H); ¹³C NMR (75 MHz, CDCl₃) δ 173.5, 166.6, 157.5, 140.7, 139.2, 136.8, 134.2, 133.5, 133.1, 132.6, 130.8, 130.5, 129.2, 127.4, 126.6, 124.4, 118.6, 112.5, 61.4, 52.1, 51.4, 33.2, 28.9, 24.7, 16.0; ESIMS m/z (rel intensity) 407.58 (MH⁺, 46). Anal. (C₂₄H₂₅NO₅) C, H, N.

(E)-3-Chloro-5-[1-(3-cyanophenyl)-5-methoxycarbonyl-pent-1-enyl]-2-methoxybenzoic Acid Methyl Ester (17). The general procedure was followed using the vinylstannane 44 (247 mg, 0.477 mmol), iodide 55 (246.7 mg, 0.756 mmol), cesium fluoride (220 mg, 1.45 mmol) and Pd(PBu^{t}₃)₂ (25.2 mg, 0.048 mmol) in toluene (1 mL). The mixture was stirred at room temperature for 18 h, at 60° C. for 23.5 h and at 110° C. for 23 h. The residue was purified by column chromatography on silica gel (15 g), eluting with EtOAc-hexanes (0-5%) to afford the product 17 (60 mg) as an oil in 29% yield. IR (KBr) 2952, 2230, 1736, 1597, 1576, 1477, 1436, 1403, 1362, 1303, 1264, 1201, 1162, 1095, 999, 969, 884, 849, 800, 744, 706, 634 cm⁻¹; ¹H NMR (300 MHz, CDCl₃) δ 7.58 (dt, J=1.5 Hz, J=7.8 Hz, 1H), 7.45 (t, J=7.6 Hz, 1H), 7.40-7.37 (m, 2H), 7.32 (dt, J=1.5 Hz, J=7.8 Hz, 1H), 7.20 (m, 1H), 6.04 (t, J=7.5 Hz, 1H), 3.86 (s, 3H), 3.84 (s, 3H), 3.57 (s, 3H), 2.23 (t, J=7.2 Hz, 2H), 2.04 (q, J=7.5 Hz, 2H), 1.76-1.66 (m, 2H); ¹³C NMR (75 MHz, CDCl₃) δ 173.5, 165.7, 154.9, 140.0, 138.3, 138.1, 134.2, 133.1, 132.3, 132.0, 131.2, 129.5, 128.0, 126.7, 118.5, 112.9, 62.0, 52.5, 51.6, 33.3, 29.1, 24.7; ESIMS m/z (rel intensity) 450.13/452.11 (MNa⁺, 86/33), 427.94/429.94 (M⁺, 37/12). Anal. (C₂₃H₂₂ClNO₅) C, H, Cl, N.

(E)-6-(5-Chloro-2-methoxyphenyl)-6-(3-cyanophenyl)-hex-5-enoic Acid Methyl Ester (18). The general procedure was followed using the vinylstannane 44 (250 mg, 0.48 mmol), bromide 47 (185 mg, 0.835 mmol), cesium fluoride (235 mg, 1.532 mmol) and Pd(PBu^{t}₃)₂ (27.6 mg, 0.053 mmol) in toluene (1 mL). The mixture was stirred at room temperature for 13 h, at 60° C. for 23.5 h and at 110° C. for 9.5 h. The residue was purified by column chromatography on silica gel (15 g), eluting with EtOAc-hexanes (0-5%) to afford the product 18 (67 mg) as an oil in 38% yield. IR (KBr) 2949, 2230, 1735, 1644, 1484, 1319, 1240, 1127, 1027, 807, 707 cm⁻¹; ¹H NMR (300 MHz, CDCl₃) δ 7.52-7.49 (m, 1H), 7.42-7.37 (m, 3H), 7.21 (dd, J=2.4 Hz, J=8.4 Hz, 1H), 7.15 (d, J=2.4 Hz, 1H), 6.71 (d, J=8.7 Hz, 2H), 5.84 (t, J=7.5 Hz, 1H), 3.62 (s, 3H), 3.52 (s, 3H), 2.30 (t, J=7.5 Hz, 2H), 2.23-2.15 (q, J=7.2-7.8 Hz, 2H), 1.82-1.72 (m, 2H); ¹³C NMR (75 MHz, CDCl₃) δ 173.6, 155.5, 141.4, 137.4, 133.4, 132.5, 130.3, 128.6, 125.4, 118.9, 112.5, 111.9, 55.6, 51.5, 33.3, 28.5, 24.8; ESIMS m/z (rel intensity) 391.95/393.96 (MNa⁺, 39/12). Anal. (C₂₁H₂₀ClNO₃) C, H, Cl, N.

(Z)-6-(3-Fluoro-5-trifluoromethylphenyl)-6-(3-methoxy-7-methylbenzo[d] isoxazol-5-yl)-hex-5-enoic Acid Methyl Ester (19). The general procedure was followed using the vinylstannane 43 (305 mg, 0.526 mmol), iodide 59 (200 mg, 0.692 mmol), cesium fluoride (245 mg, 1.6 mmol) and Pd(PBu^{t}₃)₂ (28 mg, 0.054 mmol) in toluene (1 mL). The mixture was stirred at room temperature for 19 h, at 60° C. for 8 h and at 100° C. for 22 h. The residue was purified by column chromatography on silica gel (20 g), eluting with EtOAc-hexanes (0-2%) to afford the product 19 (112 mg) as an oil in 47% yield. IR (KBr) 2949, 1736, 1599, 1549, 1496, 1438, 1376, 1329, 1220, 1171, 1130, 1047, 934, 874, 765, 701 cm⁻¹; ¹H NMR (300 MHz, CDCl₃) δ 7.29 (d, J=7.8 Hz, 1H), 7.20 (s, 1H), 7.13 (d, J=8.4 Hz, 2H), 7.05 (d, J=8.7 Hz, 1H), 6.06 (t, J=7.5 Hz, 1H), 4.12 (s, 3H), 3.62 (s, 3H), 2.45 (s, 3H), 2.30 (t, J=7.5 Hz, 2H), 2.17-2.09 (q, J=7.2-7.5 Hz, 2H), 1.84-1.74 (m, 2H); ¹³C NMR (75 MHz, CDCl₃) δ 173.6, 167.3, 164.0, 162.8, 160.7, 143.3, 139.7, 137.3, 132.8, 130.9, 130.2, 122.3, 120.8, 120.4, 120.1, 116.6, 113.8, 111.9, 111.6, 57.3, 51.5, 33.3, 29.1, 24.8, 14.7; ESIMS m/z (rel intensity) 451.97 (MH⁺, 100). Anal. (C₂₃H₂₁F₄NO₄) C, H, F, N.

(E)-6-(3-Cyanophenyl)-6-(3-methoxy-7-methylbenzo[d]isoxazol-5-yl)-hex-5-enoic Acid Methyl Ester (20). The general procedure was followed using the vinylstannane 44 (232 mg, 0.448 mmol), iodide 59 (195 mg, 0.675 mmol), cesium fluoride (245 mg, 1.6 mmol) and Pd(PBu^{t}₃)₂ (22 mg, 0.043 mmol) in toluene (1 mL). The mixture was stirred at room temperature for 19 h, at 60° C. for 8 h and at 100° C. for 43 h. The residue was purified by column chromatography on silica gel (20 g), eluting with EtOAc-hexanes (0-20%) to afford the product 20 (72 mg) as an oil in 41% yield. IR (KBr) 2949, 2230, 1738, 1615, 1548, 1495, 1394, 1315, 1169, 1047, 910, 804, 765, 695 cm⁻¹; ¹H NMR (300 MHz, CDCl₃) δ 7.61 (dt, J=1.5 Hz, J=7.8 Hz, 1H), 7.48 (t, J=7.48 Hz, 1H), 7.43 (br, 1H), 7.38 (dt, J=1.5 Hz, J=7.8 Hz, 1H), 7.13 (br, 1H), 7.09 (br, 1H), 6.06 (t, J=7.5 Hz, 1H), 4.11 (s, 3H), 3.61 (s, 3H), 2.43 (s, 3H), 2.29 (t, J=7.5 Hz, 2H), 2.14-2.07 (q, J=7.2-7.8 Hz, 2H), 1.82-1.72 (m, 2H); ¹³C NMR (75 MHz, CDCl₃) δ 173.6, 167.3, 162.8, 141.2, 139.9, 137.5, 134.3, 133.2, 130.9, 130.6, 130.2, 129.3, 120.8, 118.6, 116.6, 113.7, 112.7, 57.3, 51.6, 33.3, 29.1, 24.9, 14.7; ESIMS m/z (rel intensity) 391.03 (MH⁺, 100). Anal. (C₂₃H₂₂N₂O₄) C, H, N.

(Z)-6-(5-Chloro-2-methoxyphenyl)-6-(2,3-dihydro-2,7-dimethyl-3-oxo-benzo[d]isoxazol-5-yl)-hex-5-enoic Acid Methyl Ester (21). The general procedure was followed using the vinylstannane 51 (360 mg, 0.645 mmol), iodide 58 (272 mg, 0.941 mmol), cesium fluoride (349 mg, 2.27 mmol) and Pd(PBu^{t}₃)₂ (36 mg, 0.07 mmol) in toluene (1 mL). The mixture was stirred at room temperature for 22 h, at 60° C. for 23.5 h and at 110° C. for 23.4 h. The residue was purified by column chromatography on silica gel (25 g), eluting with EtOAc-hexanes (0-20%) to afford the product 21 (91 mg) as an oil in 33% yield. ¹H NMR (300 MHz, CDCl₃) δ 7.17 (d, 2H), 7.13 (dd, J=2.7 Hz, J=9.0 Hz, 1H), 6.87 (d, J=2.7 Hz, 1H), 6.72 (d, J=9.0 Hz, 1H), 5.97 (t, J=7.5 Hz, 1H), 3.53 (s, 3H), 3.50 (s, 3H), 3.48 (s, 3H), 2.19 (s, 3H), 2.12 (t, J=7.5 Hz, 2H), 1.90-1.84 (m, 2H), 1.66-1.56 (m, 2H); ESIMS m/z (rel intensity) 452.09 (MNa⁺, 88), 454.09 (MNa⁺, 28). Anal. (C₂₃H₂₄ClNO₅) C, H, Cl, N.

(Z)-6-(2,3-Dihydro-2,7-dimethyl-3-oxo-benzo[d]isoxazol-5-yl)-6-(3-fluoro-5-trifluoromethylphenyl)-hex-5-enoic Acid Methyl Ester (22). The general procedure was followed using the vinylstannane 43 (310 mg, 0.535 mmol), the iodide 58 (239 mg, 0.827 mmol), cesium fluoride (262 mg, 1.71 mmol) and Pd(PBu^{t}₃)₂ (29 mg, 0.056 mmol) in toluene (1 mL). The mixture was stirred at room temperature for 18 h, at 60° C. for 8 h and at 100° C. for 45 h. The residue was purified by column chromatography on silica gel (20 g), eluting with EtOAc-hexanes (0-20%) to afford the product 22 (101 mg) as a white solid in 42% yield. The solid was recrystallized with ethyl acetate and hexanes to afford a colorless needle crystal for X-ray crystallography: mp 97.5-98.5° C. IR (KBr) 2952, 1738, 1694, 1616, 1599, 1492, 1470, 1438, 1377, 1318, 1228, 1171, 1131, 1090, 1001, 934, 875, 854, 772, 786, 712, 698, 631 cm⁻¹; ¹H NMR (300 MHz, CDCl₃) δ 7.35 (d, J=1.2 Hz, 2H), 7.28 (dt, J=8.4 Hz, 1H), 7.21 (br, 1H), 7.17 (br, 1H), 7.02 (dt, J=8.7 Hz, 1H), 6.54 (s, 1H), 6.06 (t, J=7.5 Hz, 1H), 3.64 (s, 3H), 3.60 (s, 3H), 2.33 (s, 3H), 2.29 (t, J=7.5 Hz, 2H), 2.15-2.08 (q, J=7.2-7.5 Hz, 2H), 1.82-1.72 (m, 2H); ¹³C NMR (75 MHz, CDCl₃) δ 173.6, 164.1, 162.9, 158.3, 143.0, 139.3, 137.5, 132.9, 131.2, 122.3, 120.3, 120.0, 116.1, 111.7, 51.5, 33.3, 32.7, 29.1, 24.8, 14.1; ESIMS m/z (rel intensity) 451.99 (MH⁺, 100). Anal. (C₂₃H₂₁F₄NO₄) C, H, F, N.

Determination of the Structure of (Z)-6-(2,3-Dihydro-2,7-dimethyl-3-oxo-benzo[d]isoxazol-5-yl)-6-(3-fluoro-5-trifluoromethylphenyl)-hex-5-enoic Acid Methyl Ester (22) by X-ray Crystallography. DATA COLLECTION: A colorless needle of C₂₃H₂₁F₄NO₄ having approximate dimensions of 0.44×0.29×0.13 mm was mounted on a glass fiber in a random orientation. Preliminary examination and data collection were performed Mo K_{α} radiation (λ=0.71073 Å on a Nonius KappaCCD equipped with a graphite crystal, incident beam monochromator.

Cell constants for data collection were obtained from least-squares refinement, using the setting angles of 12884 reflections in the range 2<θ<27°. The triclinic cell parameters and calculated volume are: a=8.8822(7), b=9.7444(9), c=12.9675(17) Å, a=76.306(4), b=72.480(6), g=89.873(5)°, V=1036.98(19) Å³. For Z=2 and F.W.=451.42 the calculated density is 1.45 g/cm³. The refined mosaicity from DENZO/SCALEPACK was 0.39° indicating good crystal quality. The space group was determined by the program ABSEN. See, McArdle, P. “ABSEN-a PC Computer Program for Listing Systematic Absences and Apace-Group Determination” J. Appl. Cryst., 1996, 29(3), 306, the disclosure of which is hereby incorporated herein by reference. There were no systematic absences; the space group was determined to be P-1 (#2). The data were collected at a temperature of 150 (1)K. Data were collected to a maximum 20 of 55.80.

DATA REDUCTION: A total of 12884 reflections were collected, of which 4884 were unique. Lorentz and polarization corrections were applied to the data. The linear absorption coefficient is 1.2/cm for Mo Kₐ radiation. An empirical absorption correction using SCALEPACK was applied. See, Otwinowski, Z.; Minor, W. “Processing of X-Ray Diffraction Data Collected in Oscillation Mode” Methods Enzymol., 1997, 276, 307-326, the disclosure of which is incorporated herein by reference. Transmission coefficients ranged from 0.940 to 0.986. Intensities of equivalent reflections were averaged. The agreement factor for the averaging was 3.8% based on intensity.

STRUCTURE SOLUTION AND REFINEMENT: The structure was solved by direct methods using SIR2002. See, Beurskens, P. T.; Beurskens, G.; deGelder, S.; Garcia-Granda, R.; Gould, R. O.; Israel R.; Smits, J. M. M. The DIRDIF-99 Program System. Crystallography Laboratory, Univ. of Nijmegen, The Netherlands, 1999, the disclosure of which is incorporated herein by reference. The remaining atoms were located in succeeding difference Fourier syntheses. Hydrogen atoms were included in the refinement but restrained to ride on the atom to which they are bonded. The structure was refined in full-matrix least-squares where the function minimized was Σw(|Fo|²−|Fc|²)² and the weight w is defined as 1/[σ²(Fo²)+(0.0722P)²+0.0000P] where P=(Fo²+2Fc²)/3. Scattering factors were taken from the “International Tables for Crystallography”.³² 4884 reflections were used in the refinements. However, only the 3461 reflections with Fₒ²>2σ(Fₒ²) were used in, calculating R1. The final cycle of refinement included 292 variable parameters and converged (largest parameter shift was <0.01 times its su) with unweighted and weighted agreement factors of:

R1=Σ|Fo−Fc|/ΣFo=0.046

R2=SQRT(Σw(Fo²−Fc²)²/Σw(Fo²)₂)=0.117

The standard deviation of an observation of unit weight was 1.03. The highest peak in the final difference Fourier had a height of 0.27 e/A³. The minimum negative peak had a height of −0.36 e/A³. Refinement was performed on a LINUX PC using SHELX-97. See, Sheldrick, G. M. SHELEX97, Program for Crystal Structure Refinement.; University of Göttingen: Germany, 1997, the disclosure of which is incorporated herein by reference. Crystallographic drawings were done using programs ORTEP (See, C. K. Johnson, ORTEPII, Report ORNL-5138, Oak Ridge National Laboratory, Tennessee, USA, 1976, the disclosure of which is incorporated herein by reference.) and PLUTON (See, A. L. Spek, PLUTON. Molecular Graphics Program. Univ. of Ultrecht, The Netherlands, 1991, the disclosure of which is incorporated herein by reference.).

Butynyl-1-tosylate (24). Method I: A mixture of p-toluenesulfonyl chloride (62.21 g, 0.323 mol) and pyridine (31 mL, 0.383 mol) was warmed to get a colorless solution, and then cooled to get small crystals. 3-Butyn-1-ol (23) (23 mL, 0.29 mol) was added dropwise by syringe during about 20 min with stirring at 15° C. The resulting mixture was stirred below 20° C. under nitrogen atmosphere for 20 h. Water was added with cooling. The mixture was extracted with ethyl acetate (4×120 mL). The organic solution was washed with 5% aqueous sulfuric acid (3×120 mL), water (100 mL), 10% aqueous sodium hydrogen carbonate, brine, dried over sodium sulfate and concentrated. The crude product was purified by flash column chromatography on silica gel (800 g), eluting with ethyl acetate-hexanes (0-10%) to afford the tosylate 24 (56.13 g) as colorless oil in 85% yield.^{11 1}H NMR (300 MHz, CDCl₃) δ 7.79 (d, J=8.4 Hz, 2H), 7.33 (d, J=8.1 Hz, 2H), 4.08 (t, J=6.9-7.2 Hz, 2H), 2.54 (dt, J=2.7 Hz, J=6.9-7.2 Hz, 2H), 2.43 (s, 3H), 1.94 (t, J=2.7 Hz, 1H).

Method II: A solution of sodium hydroxide (22.53 g, 0.563 mol) in water (200 mL) was added to a mixture of 3-butyn-1-ol (23) (26.76 g, 0.370 mol) and p-toluenesulfonyl chloride (86.3 g, 0.448 mol) in THF (500 mL). The resulting mixture was stirred at room temperature for 50.5 h and concentrated to remove the organic solvent. The residue was extracted with ethyl acetate (3×100 mL). The combined organic solution was washed with brine, dried over Na₂SO₄ and concentrated to afford a residue. The residue was purified by column chromatography on silica gel (200 g), eluting with 0-10% EtOAc in hexanes, to afford the tosylate 24 (68.70 g) as an oil in 77% yield.

3-But-3-ynyl-1,3-oxazolidin-2-one (25). A flask was charged with 2-oxazolidinone (1.721 g, 19.17 mmol), tetrabutylammonium bromide (715 mg, 2.20 mmol), potassium carbonate (19.09 g, 138 mmol) and toluene (90 mL). 3-Butynyl-1-tosylate (24) (11.72 g, 52.26 mmol) was added. After the resulting mixture was stirred at 100° C. for 4 h, more tosylate 24 (10.62 g, 47.35 mmol) was added. After stirring overnight, more potassium carbonate (7.52 g, 54.41 mmol) and tosylate 24 (11.94 g, 53.24 mmol) were added. The resulting mixture was stirred at 105° C. for 5.5 h, and potassium carbonate (10.00 g, 72.35 mmol) and tosylate 24 (11.312 g, 50.44 mmol) were added. The mixture was heated at 105° C. overnight, and more tosylate 24 (10.70 g, 47.71 mmol) and potassium carbonate (9.29 g, 67.22 mmol) were added. The mixture was heated to reflux for another 5 h and then cooled to room temperature. Water was added to quench the reaction. The mixture was extracted ethyl acetate (4×200 mL). The organic solution was washed with brine, dried over Na₂SO₄ and concentrated to afford a residue, which was purified by column chromatography on silica gel (40 g), eluting with EtOAc-hexanes (0-30%), to afford the product 25 as an oil (2.197 g) in 83% yield. IR (KBr film) 3286, 2919, 2118, 1744, 1485, 1429, 1366, 1271, 1236, 1093, 1042, 970, 763 cm⁻¹; ¹H NMR (300 MHz, CDCl₃) δ 4.32 (t, J=7.8 Hz, 2H), 3.70 (t, J=7.8 Hz, 2H), 3.42 (t, J=6.6 Hz, 2H), 2.45 (dt, J=2.7 Hz, J=6.6 Hz, 2H), 2.00 (t, J=2.7 Hz, 1H); ¹³C NMR (75 MHz, CDCl₃) δ 157.9, 80.6, 70.0, 61.5, 44.5, 42.5, 17.4; ESIMS m/z (rel intensity) 139.98 (100). Anal. (C₇H₉NO₂) C, H, N.

2-Hydroxy-5-iodo-3-methylbenzoic Acid Methyl Ester (28). Tetrabutylammonium bromide (0.9 g, 2.764 mmol) was added to a stirred solution of 3-methylsalicylic acid 26 (4.634 g, 29.85 mmol) in dichloromethane (50 mL). A solution of potassium carbonate (13.433 g, 97.34 mmol) in water (25 mL) was added. Dimethyl sulfate (6.0 mL, 62.77 mmol) was added to afford a clear solution. The resulting solution was stirred at room temperature for 4 h. The organic layer was separated and the aqueous layer was diluted with water (40 mL) and extracted with dichloromethane (2×20 mL). The combined organic solutions were washed with sat. ammonium chloride (30 mL), brine (2×30 mL), dried over sodium sulfate and concentrated to afford crude methyl ester 27 as an oil. ¹H NMR (300 MHz, CDCl₃) δ 10.99 (s, 1H), 7.67 (dd, J=1.5 Hz, J=8.1 Hz, 1H), 7.30 (d, J=7.5 Hz, 1H), 6.76 (t, J=7.5 Hz, 1H), 3.94 (s, 3H), 2.25 (s, 3H). The crude methyl ester 27 was dissolved in methanol (80 mL), sodium iodide (5.526 g, 36.86 mmol) and sodium hydroxide (1.489 g, 36.86 mmol) were added, and the solution was cooled to 0° C. Aqueous sodium hypochlorite (62.5 mL, 36.86 mmol, 24%) was added dropwise. The resulting brown mixture was stirred for 4.5 h at 0-3° C. and then treated with 10% sodium thiosulfate (60 mL). The pH of the mixture was adjusted to 5-6 using 1 N HCl. Ether (200 mL) was added and the layers were separated. The aqueous layer was extracted with ether (3×200 mL). The combined organic solution was washed with brine (300 mL), dried over anhydrous Na₂SO₄ and concentrated to afford crude 28 (8.52 g) as colorless crystals in 98% yield: mp 94-95.5° C. (lit. mp 82-84° C.). ¹H NMR (300 MHz, CDCl₃) δ 10.80 (s, 1H), 7.83 (d, J=2.4 Hz, 1H), 7.43 (d, J=2.4 Hz, 1H), 3.80 (s, 3H), 2.07 (s, 3H).

Methyl 5-Iodo-2-methoxy-3-methylbenzoate (29). The methyl ester (28) (8.52 g, 29.17 mmol) was dissolved in dichloromethane (100 mL). Then tetrabutylammonium bromide (958 mg, 2.94 mmol) was added. A solution of sodium hydroxide (3.4 g, 85 mmol) in water (50 mL) was added, followed by dimethyl sulfate (5.5 mL, 57.55 mmol). The resulting solution was stirred at room temperature overnight and quenched with solid ammonium chloride (6 g), and the pH was adjust to 5-6 with 1 N HCl. The organic layer was separated and the aqueous layer was extracted with dichloromethane (3×100 mL). The combined organic solution was washed with brine (200 mL), dried over sodium sulfate and concentrated to afford a solid, which was purified by column chromatography on silica gel (40 g) using hexanes and 5% ethyl acetate in hexanes to afford 29 (7.69 g) as white crystals in 86% yield: mp 66-67.5° C. (lit. mp 55-57° C.). ¹H NMR (300 MHz, CDCl₃) δ 7.93 (d, J=2.4 Hz, 1H), 7.66 (d, J=2.4 Hz, 1H), 3.90 (s, 3H), 3.81 (s, 3H), 2.27 (s, 3H).

2-Methoxy-3-methyl-5-[4-(2-oxo-oxazolidin-3-yl)-but-1-ynyl]benzoic Acid Methyl Ester (30). Triethylamine (1.35 mL, 9.69 mmol) and Pd(PPh₃)Cl₂ (139 mg, 0.19 mmol) were added to a mixture of the iodide 29 (1.310 g, 4.28 mmol) and the alkyne 25 (536 mg, 3.86 mmol) in THF (25 mL) at room temperature, and then Cu(I)I (75 mg, 0.40 mmol) was added. The resulting mixture was stirred at room temperature for 22 h. The reaction was quenched with water (20 mL) and concentrated to remove the organic solvents. The residue was extracted with ethyl acetate (3×50 mL). The organic layer was separated and washed with brine (60 mL), dried over sodium sulfate and concentrated. The residue was purified by column chromatography on silica gel (35 g), eluting with EtOAc-hexanes (0-50%) to afford the product 30 (981 mg) as brown oil in 80% yield. ¹H NMR (300 MHz, CDCl₃) δ 7.63 (d, J=2.1 Hz, 1H), 7.34 (d, J=1.5 Hz, 1H), 4.33 (t, J=7.8 Hz, 2H), 3.89 (s, 3H), 3.80 (s, 3H), 3.73 (t, J=7.8 Hz, 2H), 3.50 (t, J=6.6 Hz, 2H), 2.66 (t, J=6.6 Hz, 2H), 2.26 (s, 3H); ESIMS m/z (rel intensity) 340.10 (MNa⁺, 100). Anal. Calcd for (C₁₇H₁₉NO₅) C, H, N.

2-Methoxy-3-methyl-5-[1-(tributylstannanyl)-4-(2-oxo-oxazolidin-3-yl)-but-1-enyl]benzoic Acid Methyl Ester (31). Compound 30 (945 mg, 0.945 mmol) was dissolved in THF (20 mL), and then tetrakis(triphenylphosphine)palladium (7.0 mg, 6.05 μmol) was added. The mixture was cooled to 0° C., degassed by gently bubbling argon through for 15 min, and then tributyltin hydride (0.4 mL, 1.44 mmol) was added dropwise over 60 min. After the mixture was stirred at room temperature for 3 h, it was concentrated to yield a residue. The residue was purified by column chromatography on silica gel (40 g) using hexanes and EtOAc-hexanes (5-15%) to afford the vinylstannanes 31 (437 mg) as an oil in 76% yield and 36 (53 mg) in 9% yield. Spectral data of 31: IR (KBr) 2926, 1756, 1435, 1257, 1197, 1126 cm⁻¹; ¹H NMR (300 MHz, CDCl₃) δ 7.15 (d, J=2.4 Hz, 1H), 6.86 (d, J=2.1 Hz, 1H), 5.71 (t, J=6.9 Hz, 1H), 4.22 (t, J=7.8 Hz, 2H), 3.87 (s, 3H), 3.79 (s, 3H), 3.34 (t, J=7.8 Hz, 2H), 3.27 (t, J=7.2 Hz, 2H), 2.26 (m, 5H), 1.42-1.34 (m, 6H), 1.29-1.17 (m, 6H), 0.88-0.78 (m, 9H); ESIMS m/z (rel intensity) 633.33 (MNa⁺, 77), 632.20 (MNa⁺, 100). Anal. (C₂₉H₄₇NO₅Sn) C, H, N, Sn.

2-Methoxy-3-methyl-5-[2-(tributylstannanyl)-4-(2-oxo-oxazolidin-3-yl)-but-1-enyl]benzoic Acid Methyl Ester (36). IR (KBr) 2955, 2926, 2871, 1756, 1732, 1480, 1424, 1377, 1360, 1315, 1263, 1232, 1201, 1128, 1102, 1051, 1010, 881, 799, 762, 695 cm¹; ¹H NMR (300 MHz, CDCl₃) δ 7.47 (d, J=2.1 Hz, 1H), 7.24 (d, J=2.1 Hz, 1H), 6.61 (s, 1H), 4.21 (t, J=7.8 Hz, 2H), 3.90 (s, 3H), 3.81 (s, 3H), 3.42 (t, J=7.8 Hz, 2H), 3.30 (t, J=7.2 Hz, 2H), 2.70 (t, J=7.2 Hz, 2H), 2.31 (s, 3H), 1.58-1.47 (m, 6H), 1.39-1.27 (m, 6H), 1.00-0.96 (m, 6H), 0.89 (t, J=7.2 Hz, 9H); ESIMS m/z (rel intensity) 552.17 ([M-Bu]⁺, 38.5), 608.09 (M⁺, 12), 610.07 (M⁺, 18). Anal. (C₂₉H₄₇NO₅Sn) C, H, N, Sn.

5-[1-Iodo-4-(2-oxo-oxazolidin-3-yl)-but-1-enyl]-2-methoxy-3-methylbenzoic acid Methyl Ester (32). The vinyl tributylstannane 31 (157 mg, 0.258 mmol) was dissolved in dry CH₂Cl₂ (6 mL). Finely divided 12 (80 mg, 0.315 mmol) was added, and the mixture was stirred vigorously at room temperature for 50 min. Saturated aqueous Na₂S₂O₃ (15 mL) was added, the phases were separated, and the aqueous phase was extracted with CH₂Cl₂ (3×10 mL). The combined organic extracts were dried (Na₂SO₄) and concentrated. The residue was purified by flash chromatography on silica gel (20 g, 30% EtOAc-hexanes as eluent) to afford the vinyl iodide as an oil (114 mg, 99%): ¹H NMR (CDCl₃) δ 7.46 (d, J=2.1 Hz, 1H), 7.20 (d, J=1.5 Hz, 1H), 6.40 (t, J=7.5 Hz, 1H), 4.23 (t, J=7.8 Hz, 2H), 3.86 (s, 3H), 3.78 (s, 3H), 3.36 (t, J=7.8 Hz, 2H), 3.24 (t, J=6.9 Hz, 2H), 2.25 (s, 3H), 2.19 (t, J=7.2 Hz, 2H); ¹³C NMR (CDCl₃) δ 166.1, 158.2, 158.0, 139.4, 136.5, 134.9, 133.1, 128.8, 124.1, 95.5, 61.5, 61.4, 52.2, 44.5, 43.1, 30.1, 16.0; ESIMS m/z (rel intensity) 467.85 (MNa⁺, 100). Anal. (C₁₇H₂₀INO₅) C, H, I, N.

3-[4-(3,4-Dimethoxyphenyl)-but-3-ynyl]-1,3-oxazolidin-2-one (34). Triethylamine (0.08 mL, 0.574 mmol) and Pd(PPh₃)Cl₂ (8.0 mg, 0.011 mmol) were added to a mixture of 3,4-dimethoxyphenyl iodide (33) (64 mg, 0.242 mmol) and alkyne 25 (34 mg, 0.245 mmol) in THF (3 mL) at room temperature. Then Cu(I)I (5 mg, 0.026 mmol) was added. The resulting mixture was stirred at room temperature for 4 h. The reaction was quenched with water (10 mL) and concentrated to remove the organic solvents. The residue was diluted with ethyl acetate (15 mL). The organic layer was separated and washed with brine (2×10 mL), dried over sodium sulfate and concentrated. The residue was purified by column chromatography on silica gel (25 g), eluting with EtOAc-hexanes (10-50%) to afford the product 34 (54 mg) as solid in 80% yield: mp 78-79° C. ¹H NMR (300 MHz, CDCl₃) δ 6.95 (dd, J=2.1 Hz, J=8.4 Hz, 1H), 6.88 (d, J=1.8 Hz, 1H), 6.76 (d, J=8.4 Hz, 1H), 4.32 (t, J=7.8 Hz, 2H), 3.86 (s, 3H), 3.85 (s, 3H), 3.73 (t, J=7.8 Hz, 2H), 3.51 (t, J=6.6 Hz, 2H), 2.67 (t, J=6.6 Hz, 2H); ESIMS m/z (rel intensity) 276.05 (MH⁺, 94), 298.05 (MNa⁺, 98). Anal. (C₁₅H₁₇NO₄) C, H, N.

3-[4-(Tributylstannanyl)-4-(3,4-dimethoxyphenyl)-but-3-enyl]-1,3-oxazolidin-2-one (35). The intermediate 34 (254 mg, 0.923 mmol) was dissolved in THF (20 mL), and then tetrakis(triphenylphosphine)palladium (9.4 mg, 8.13 μmol) was added. The mixture was cooled to 0° C., degassed by gently bubbling argon through for 15 min and then tributyltin hydride (0.4 mL, 1.44 mmol) was added dropwise over 70 min. After the mixture was stirred at room temperature for 3.5 h, it was concentrated to yield a residue. The residue was purified by column chromatography on silica gel (30 g) using hexane and EtOAc-hexane (0-30%) to afford the vinylstannanes 35 (415 mg) in 79% yield and 37 as an oil (39.2 mg) in 8% yield. Spectral data of 35: IR (KBr) 2954, 2926, 1755, 1508, 1463, 1254, 1233, 1136, 1028, 865, 761 cm⁻¹; ¹H NMR (300 MHz, CDCl₃) δ 6.77 (m, 1H), 6.45-6.42 (m, 2H), 5.68 (t, J=6.9 Hz, 1H), 4.19 (t, J=8.1 Hz, 2H), 3.84 (s, 3H), 3.82 (s, 3H), 3.32 (t, J=8.1 Hz, 2H), 3.27 (t, J=7.2 Hz, 2H), 2.32 (m, 2H), 1.49-1.38 (m, 6H), 1.34-1.18 (m, 6H), 0.95-0.76 (m, 14H); ¹³C NMR (75 MHz, CDCl₃) δ 158.1, 148.6, 148.4, 146.4, 137.1, 136.8, 118.5, 111.0, 110.1, 61.4, 55.7, 55.6, 44.1, 43.8, 28.8, 27.1, 13.5, 9.8; ESIMS m/z (ret intensity) 588.45 (MNa⁺, 80.4), 590.19 (MNa⁺, 100). Anal. (C₂₇H₄₅NO₄Sn) C, H, N, Sn.

3-[3-(Tributylstannanyl)-4-(3,4-dimethoxyphenyl)-but-3-enyl]-1,3-oxazolidine-2-one (37). ¹H NMR (300 MHz, CDCl₃) δ 6.83 (m, 2H), 6.74 (s, 1H), 6.64 (s, 1H), 4.20 (t, J=7.5-8.4 Hz, 2H), 3.89 (s, 3H), 3.87 (s, 3H), 3.38 (t, J=7.5-8.4 Hz, 2H), 3.31 (t, J=7.5-8.1 Hz, 2H), 2.74 (t, J=7.5-8.1 Hz, 2H), 1.59-1.49 (m, 6H), 1.40-1.28 (m, 6H), 1.02-0.96 (m, 6H), 0.090 (t, J=7.1 Hz, 9H); ESIMS m/z (rel intensity) 564.30 (M⁺, 12), 566.25 (M⁺, 23), 568.26 (M⁺, 28), 586.32 (MNa⁺, 42), 588.31 (MNa⁺, 77), 590.26 (MNa⁺, 100).

6-(3-Fluoro-5-trifluoromethylphenyl)-hex-5-ynoic Acid Methyl Ester (41). Pd(PPh₃)₂Cl₂ (236 mg, 0.336 mmol) was added to a mixture of bromide 38 (1.677 g, 6.70 mmol) and methyl 5-hexynoate (1.025 g, 8.12 mmol) in triethylamine (5.0 mL) at room temperature. Cu(I)I (136 mg, 0.714 mmol) was added. The resulting mixture was stirred at room temperature for 1.5 h and at 80° C. for 22.5 h. The reaction mixture was cooled to room temperature, filtered through a short column of silica gel (5 g), and the column was washed with ethyl acetate. The organic solution was concentrated. The residue was purified by column chromatography on silica gel (40 g), eluting with EtOAc-hexanes (2%) to afford the product 41 (1.564 g) as a white solid in 81% yield: mp 44-45° C. IR (KBr) 3084, 2955, 2848, 2238, 1740, 1619, 1599, 1467, 1439, 1363, 1253, 1240, 1224, 1171, 1133, 1093, 1046, 995, 973, 924, 911, 875, 695 cm⁻¹; ¹H NMR (300 MHz, CDCl₃) δ 7.40 (s, 1H), 7.21 (m, 2H), 3.66 (s, 3H), 2.47 (t, J=7.2 Hz, 4H), 1.90 (m, 2H); ¹³C NMR (75 MHz, CDCl₃) δ 173.3, 163.7, 160.4, 132.8, 132.3, 126.8, 126.7, 124.7, 124.3, 121.8, 121.5, 112.3, 111.9, 79.1, 51.6, 32.7, 23.5, 18.7; ESIMS m/z (rel intensity) 288.96 (MH⁺, 51). Anal. (C₁₄H₁₂F₄O₂) C, H, F.

6-(3-Cyanophenyl)-hex-5-ynoic Acid Methyl Ester (42). Pd(PPh₃)₂Cl₂ (223 mg, 0.315 mmol) was added to a mixture of 3-bromobenzonitrile (40) (832 mg, 6.43 mmol) and methyl 5-hexynoate (970 mg, 7.69 mmol) in triethylamine (4.5 mL) at room temperature, and then Cu(I)I (122 mg, 0.64 mmol) was added. The resulting mixture was stirred at room temperature for 1 h and at 80° C. for 22 h. The reaction mixture was cooled to room temperature, filtered through a short column of silica gel (5 g), and the column washed with ethyl acetate. The organic solution was concentrated. The residue was purified by column chromatography on silica gel (30 g), eluting with EtOAc-hexanes (3-5%) to afford the product 42 (1.117 g) as an oil in 76% yield. IR (KBr) 3069, 2952, 2232, 1737, 1597, 1572, 1479, 1436, 1416, 1370, 1316, 1222, 1160, 1057, 894, 799, 684 cm⁻¹; ¹H NMR (300 MHz, CDCl₃) δ7.64 (m, 1H), 7.58 (dt, J=1.2 Hz, J=7.8 Hz, 1H), 7.53 (dt, J=1.2 Hz, J=7.8 Hz, 1H), 7.38 (t, J=7.5 Hz, 1H), 3.68 (s, 3H), 2.48 (m, 4H), 1.92 (m, 2H); ¹³C NMR (75 MHz, CDCl₃) δ 173.4, 135.6, 134.9, 130.9, 129.1, 125.3, 118.1, 112.6, 91.7, 79.3, 51.6, 32.8, 23.6, 18.8; EIMS m/z (rel intensity) 227 (M⁺, 29), CIMS m/z (rel intensity) 228 (MH⁺, 100). Anal. (C₁₄H₁₃NO₂) C, H, N.

6-Tributylstannanyl-6-(3-fluoro-5-trifluoromethylphenyl)-hex-5-enoic Acid Methyl Ester (43). Alkyne 41 (1.545 g, 5.36 mmol) was dissolved in THF (220 mL), and then tetrakis(triphenylphosphine)palladium (56 mg, 48 μmol) was added. The mixture was cooled to 0° C., degassed by gently bubbling argon through for 20 min, and then tributyltin hydride (2.2 mL, 7.93 mmol) was added dropwise over 120 min. After the mixture was stirred at room temperature for 3 h, it was concentrated to yield a residue. The residue was purified by column chromatography on silica gel (50 g) using hexanes and EtOAc-hexanes (0-1%) to afford the vinylstannane 43 (2.651 g) as an oil in 90% yield. IR (KBr) 2957, 2928, 2873, 2854, 1743, 1617, 1593, 1464, 1434, 1369, 1327, 1246, 1224, 1170, 1133, 1090, 978, 903, 877, 865, 695, 706 cm⁻¹; ¹H NMR (300 MHz, CDCl₃) δ 7.05 (d, J=8.7 Hz, 1H), 6.92 (s, 1H), 6.76 (t, J=9.3 Hz, 1H), 5.76 (t, J=7.2 Hz, 1H), 3.59 (s, 3H), 2.23 (t, J=7.5 Hz, 2H), 2.01 (m, 2H), 1.68 (m, 2H), 1.45-1.35 (m, 6H), 1.30-1.18 (m, 6H), 0.89-0.81 (m, 9H); ¹³C NMR (75 MHz, CDCl₃) δ 173.7, 163.9, 160.6, 148.8, 148.7, 144.6, 142.2, 132.4, 132.0, 119.4, 117.0, 116.7, 109.2, 108.8, 51.4, 33.2, 29.4, 28.9, 27.2, 24.6, 13.5, 10.0; ESIMS m/z (rel intensity) 523.15 (M-Bu⁺, 64). Anal. (C₂₆H₄₀F₄O₂Sn) C, H, F, Sn.

6-(Tributylstannanyl)-6-(3-cyanophenyl)-hex-5-enoic Acid Methyl Ester (44). Alkyne 42 (787 mg, 3.46 mmol) was dissolved in THF (150 mL) and then tetrakis(triphenylphosphine)palladium (40 mg, 34.6 mmol) was added. The mixture was cooled to 0° C., degassed by gently bubbling argon through for 15 min, and then tributyltin hydride (1.5 mL, 5.41 mmol) was added dropwise over 60 min. After the mixture was stirred at 0° C. for 15 min and at room temperature for 6 h, the mixture was concentrated to yield a residue. The residue was purified by column chromatography on silica gel (20 g) using hexanes and EtOAc-hexanes (5%) to afford the vinylstannane 44 (1.696 g) as an oil in 95% yield. IR (KBr) 2955, 2927, 2871, 2853, 2230, 1740, 1607, 1590, 1571, 1474, 1457, 1436, 1417, 1376, 1313, 1292, 1247, 1217, 1161, 1074, 1048, 1074, 1022, 1000, 960, 911, 875, 841, 804, 770, 697, 677 cm⁻¹; ¹H NMR (300 MHz, CDCl₃) δ 7.37 (dt, J=1.5 Hz, J=7.8 Hz, 1H), 7.34 (q, J=7.8 Hz, 1H), 7.14 (m, 1H), 7.08 (dt, J=1.5 Hz, J=7.5 Hz, 1H), 5.75 (t, J=7.2 Hz, 1H), 3.61 (s, 3H), 2.20 (t, J=7.5 Hz, 2H), 1.97 (q, J=7.5 Hz, 2H), 1.64 (m, 2H), 1.47-1.35 (m, 6H), 1.28-1.16 (m, 6H), 0.87-0.79 (m, 9H); ¹³C-NMR (75 MHz, CDCl₃) δ 173.7, 146.4, 144.6, 142.1, 131.3, 130.0, 128.9, 128.5, 119.0, 112.1, 51.4, 33.2, 29.3, 28.8, 27.2, 24.6, 13.6, 9.9; ESIMS m/z (rel intensity) 515.96 (M⁺, 31), 517.99 (M⁺, 49), 519.98 (M⁺, 61). Anal. (C₂₆H₄₁NO₂Sn) C, H, N, Sn.

2-Bromo-4-chloro-1-methoxybenzene (47). A solution of sodium hydroxide (836 mg, 20.9 mmol) in water (8.0 mL) was added to a solution of 2-bromo-4-chlorophenol (45) (2.1 g, 9.92 mmol) and tetrabutylammonium bromide (336.4 mg, 1.03 mmol) in dichloromethane. Dimethyl sulfate (1.5 mL, 15.69 mmol) was added. The resulting mixture was stirred at room temperature for 22 h and then 1 N aq HCl (about 2 mL) was added to quench the reaction. The organic phase was separated and the aqueous phase was extracted with dichloromethane (2×15 mL). The combined organic phase was washed with brine, dried over sodium sulfate and concentrated to afford an oil. The crude product was purified by column chromatography on silica gel (10 g) using EtOAc-hexanes (5%) to afford the product 47 as a colorless oil (2.17 g) in 99% yield. See, Dischino, D. D.; Gribkoff, V. K.; Hewawasam, P.; Luke, G. M.; Rinehart, J. K.; Spears, T. L.; Starrett Jr, J. E. Synthesis of 3H and 14 C Labeled (S)-3-(5-Chloro-2-methoxyphenyl)-1,3-dihydro-3-fluoro-6-(trifluoromethyl)-2H-indol-2-one, Maxipost™. An Agent for Post-Stroke Neuroprotection. J. Label. Compd. Radiopharm. 2003, 46, 139-149, the disclosure of which is incorporated herein by reference. ¹H NMR (300 MHz, CDCl₃) δ 7.51 (d, J=2.4 Hz, 1H), 7.22 (dd, J=2.4 Hz, J=8.7 Hz, 1H), 6.80 (d, J=8.7 Hz, 1H), 3.86 (s, 3H).

Methyl 5-Bromo-2-methoxy-3-methylbenzoate (48). 5-Bromo-3-methylsalicylic acid (46) (6.6 g, 28.57 mmol), potassium carbonate (19.2 g) and acetone (150 mL) were added to an oven-dried 250 mL round-bottom flask equipped with a stirring bar. Dimethyl sulfate (8.55 mL, 88.94 mmol) was then added to the flask via syringe. The reaction mixture was heated at reflux temperature for 26 h. The mixture was cooled to room temperature, filtered, and the inorganic salts were washed with methylene chloride (20 mL). The solution was evaporated to yield the crude product. The crude product was purified by flash column chromatography on silica gel (60 g) using 3-30% ethyl acetate in hexanes as the eluent to yield the product 48 as a crystallizing oil (7.4 g) in quantitative yield: mp 57-58.5° C. (lit. mp 59-60° C.). ¹H NMR (300 MHz, CDCl₃) δ 7.76 (d, J=2.58 Hz, 1H), 7.47 (d, J=2.58 Hz, 1H), 3.91 (s, 3H), 3.81 (s, 3H), 2.30 (s, 3H).

6-(5-Chloro-2-methoxyphenyl)-hex-5-ynoic Acid Methyl Ester (49). Pd(PPh₃)₂Cl₂ (76 mg, 0.106 mmol) was added to a mixture of bromide 47 (498 mg, 2.25 mmol) and methyl 5-hexynoate (440 mg, 3.48 mmol) in triethylamine (3.0 mL) at room temperature, and then Cu(I)I (45 mg, 0.235 mmol) was added. The resulting mixture was stirred at room temperature for 8 h, and at 80° C. for 21 h. The reaction mixture was cooled to room temperature, filtered through a short column of silica gel (5 g), and the column washed with ethyl acetate. The organic solution was concentrated. The residue was purified by column chromatography on silica gel (25 g), eluting with EtOAc-hexanes (3-5%) to afford the product 49 (589 mg) as a slightly yellow oil in 98% yield. IR (KBr) 2950, 2844, 2234, 1736, 1591, 1490, 1460, 1439, 1397, 1370, 1313, 1287, 1266, 1229, 1180, 1161, 1138, 1095, 1026, 935, 882, 807, 709, 647 cm⁻¹; ¹H NMR (300 MHz, CDCl₃) δ 7.30 (d, J=2.4 Hz, 1H), 7.17 (dd, J=2.7 Hz, J=8.7 Hz, 1H), 6.74 (d, J=8.0 Hz, 1H), 3.83 (s, 3H), 3.67 (s, 3H), 2.52 (m, 4H), 1.92 (m, 2H); ESIMS m/z (rel intensity) 289.04 (MNa⁺, 4.5). Anal. (C₁₄H₁₅ClO₃) C, H, Cl.

6-Tributylstannanyl-6-(5-chloro-2-methoxyphenyl)-hex-5-enoic Acid Methyl Ester (51). Alkyne 49 (1.217 g, 4.63 mmol) was dissolved in THF (200 mL), and then tetrakis(triphenylphosphine)palladium (45 mg, 39 μmol) was added. The mixture was cooled to 0° C., degassed by gently bubbling argon through for 15 min, and then tributyltin hydride (1.9 mL, 6.85 mmol) was added dropwise over 60 min. After the mixture was stirred at 0° C. for 15 min and at room temperature for 2 h, it was concentrated to yield a residue. The residue was purified by column chromatography on silica gel (45 g) using hexanes and EtOAc-hexanes (3%) to afford the vinylstannane 51 (2.269 g) as an oil in 88% yield. IR (KBr) 2954, 2926, 2871, 2852, 1740, 1481, 1462, 1438, 1395, 1375, 1290, 1240, 1173, 1128, 1079, 1030, 878, 804 cm⁻¹; ¹H NMR (300 MHz, CDCl₃) δ 7.04 (dd, J=2.4 Hz, J=8.7 Hz, 1H), 6.80 (d, J=2.4 Hz, 1H), 6.68 (d, J=8.7 Hz, 1H), 5.70 (t, J=6.9 Hz, 1H), 3.70 (s, 3H), 3.59 (s, 3H), 2.23 (t, J=7.5 Hz, 2H), 2.04 (m, 2H), 1.66 (m, 5H), 1.40 (m, 6H), 1.29-1.17 (m, 6H), 0.85-0.76 (m, 9H); ¹³C NMR (75 MHz. CDCl₃) δ 174.1, 153.9, 142.0, 141.0, 134.9, 127.7, 125.8, 125.0, 110.9, 55.2, 51.4, 33.3, 29.5, 28.9, 27.7, 27.3, 26.9, 24.7, 13.7, 10.3; ESIMS m/z (rel intensity) 501.14 (M-Bu⁺, 100). Anal. (C₂₆H₄₃ClO₃Sn) C, H, Cl, Sn.

Methyl 6-(Tributylstannyl)-6-[4-methoxy-5-methoxycarbonyl-3-methylphenyl]-hex-5-enoate (52). Aryl bromide 48 (2.25 g, 8.68 mmol), methyl 5-hexynoate (1.329 g, 10.54 mmol) and dichlorobis(triphenylphosphine)palladium(II) (307 mg, 0.437 mmol) were added to a flask under an argon atmosphere. Triethylamine (6.5 mL) was added to the flask. Cuprous iodide (168 mg, 0.88 mmol) was then added to the flask. The reaction mixture was stirred at room temperature for 0.5 h and heated at 80° C. for 21.5 h. The reaction mixture was cooled to room temperature, filtered through a short column of silica gel (5 g), and the column was washed with ethyl acetate. The organic solution was concentrated. The residue was purified by column chromatography on silica gel (40 g), eluting with EtOAc-hexanes (2%) to afford the alkyne 50 as a crude red oil. The crude oil was dissolved in dry THF (400 mL) and added to a flask under an argon atmosphere. Tetrakis(triphenylphosphine)palladium(0) (100 mg, 0.0865 mmol) was added to the flask. The mixture was cooled to 0° C., degassed by gently bubbling argon through for 20 min, and then tributyltin hydride (3.6 mL, 12.98 mmol) was added dropwise over 110 min. After the mixture was stirred at room temperature for 2 h, it was concentrated to yield a residue. Solvent was removed in vacuo to yield a crude black oil. The oil was purified by flash column chromatography using silica gel (60 g) and a gradient eluant from 0 to 2% ethyl acetate in hexanes to yield the product 52 as a colorless oil (4.64 g, 90%, over two steps). ¹H NMR (500 MHz, CDCl₃) δ 7.15 (d, J=2.06 Hz, 1H), 6.85 (d, J=2.06 Hz, 1H), 5.69 (t, J=6.91 Hz, 1H), 3.87 (s, 3H), 3.79 (s, 3H), 3.60 (s, 3H), 2.26 (s, 3H), 2.23 (t, J=7.68 Hz, 2H), 2.04 (q, J=7.17 Hz, 2H), 1.66 (q, J=7.46 Hz, 2H), 1.45-1.20 (m, 11H), 0.86-0.69 (m, 16H).

Methyl 5-Iodo-2-methoxybenzoate (54). To a solution of 5-iodosalicyclic acid (53) (25.20 g, 90.67 mmol) in dichloromethane (200 mL) was added tetrabutylammonium bromide (3.1 g, 9.52 mmol). A solution of sodium hydroxide (14.66 g, 0.367 mol) in water (60 mL) was added, followed by an addition of dimethyl sulfate (26 mL, 0.272 mol). The resulting mixture was stirred at room temperature for 53 h. 1 N HCl was added until the pH was about 5 to quench the reaction. The organic phase was separated and the aqueous phase was extracted with dichloromethane (60 mL). The combined organic solution was washed with brine (80 mL), dried over anhydrous sodium sulfate and concentrated. The residue was purified by column chromatography on silica gel (100 g) using hexanes and 1% ethyl acetate in hexanes to afford the product 54 (20.02 g) as a white solid in 76% yield: mp 57-57.5° C. (lit.²⁵ mp 48-50° C.). ¹H NMR (300 MHz, CDCl₃) δ 8.04 (d, J=2.1 Hz, 1H), 7.70 (dd, J=8.7, 2.4 Hz, 1H), 6.73 (d, J=8.7 Hz, 1H), 3.86 (s, 6H).

Methyl 3-Chloro-5-iodo-2-methoxybenzoate (55). A mixture of the iodide 54 (4.331 g, 14.83 mmol) and SO₂Cl₂ (5.1 mL, 61.58 mmol) was heated at 50° C. for 20 h and then cooled to room temperature. It was poured into ice (20 g) and extracted with CH₂Cl₂ (3×80 mL). The CH₂Cl₂ extracts were combined, washed with brine, dried over Na₂SO₄ and evaporated in vacuo. The residue was further purified by flash chromatography on silica gel (50 g, hexanes and 3% EtOAc in hexanes as eluent) and was recrystallized with ethyl acetate and hexanes to afford white crystals (4.60 g, 95%): mp 48-48.5° C. (lit.⁷ mp 41-43° C.); ¹H NMR (CDCl₃) δ 7.97 (d, J=2.1 Hz, 1H), 7.83 (d, J=2.1 Hz, 1H), 3.91 (s, 6H).

2,N-Dihydroxy-5-iodo-3-methylbenzamide (56). A mixture of ester 28 (2.0 g, 6.86 mmol), hydroxylamine hydrochloride (967 mg, 13.74 mmol) and potassium hydroxide (1.91 g, 30.0 mmol) in methanol (40 mL) was heated to reflux for 6 h, and then cooled to room temperature. The mixture was acidified with acetic acid until the pH was about 6, and concentrated to remove the solvents. The residue was mixed with EtOAc (100 mL), and water (80 mL) was added to get a clear solution. The organic solution was separated and the aqueous solution was extracted with EtOAc (2×50 mL). The combined organic solution was washed with brine, dried over anhydrous sodium sulfate and concentrated to afford a white solid residue, which was purified by column chromatography on silica gel (35 g) using EtOAc-hexanes (0-20%) to afford the product 56 (1.688 g) in 84% yield: mp 151-152.5° C. (dec). ¹H NMR (300 MHz, acetone-d₆) δ12.60 (s, 1H), 11.12 (s, 1H), 8.61 (s, 1H), 7.81 (d, J=1.8 Hz, 1H), 7.59 (d, J=1.8 Hz, 1H), 2.20 (s, 3H); ESIMS m/z (rel intensity) 293.87 (MH⁺, 45); negative ion ESIMS m/z (rel intensity) 292.03 (M−H⁺, 100). Anal. (C₈H₈INO₃) C, H, I, N.

5-Iodo-7-methylbenzo[d]isoxazol-3-one (57). A solution of carbonyldiimidazole (2.243 g, 13.83 mmol) in THF (40 mL) was added to a boiling solution of 56 (2.022 g, 6.9 mmol) in THF (20 mL). The resulting solution was then heated under reflux for 2 h, cooled and evaporated. The residue was mixed with water to afford a precipitate. The precipitate was collected and washed with water, and then dissolved in ethyl acetate (150 mL), washed with brine (3×50 mL) dried over Na₂SO₄, and concentrated. The residue was recrystallized with ethyl acetate to afford the product 57 (1.879 g) as a white solid in 99% yield: mp 232-233° C. IR (KBr) 2927, 2760, 2672, 2613, 1726, 1702, 1608, 1564, 1510, 1465, 1384, 1345, 1296, 1260, 1198, 1176, 1122, 1019, 967, 742, 864, 806, 763, 721, 612, 559, 503 cm⁻¹; ¹H NMR (300 MHz, DMSO-d₆) δ 12.22 (bs, 1H), 7.90 (s, 1H), 7.71 (s, 1H), 2.39 (s, 3H); ESIMS m/z (rel intensity) 275.95 (MH⁺, 28); negative ion ESIMS m/z (rel intensity) 274.10 (M−H⁺, 100). Anal. (C₈H₆₁NO₂) C, H, I, N.

5-Iodo-2,7-dimethyl-benzo[d]isoxazol-3-one (58) and 5-Iodo-3-methoxy-7-methylbenzo[d]isoxazole (59). Iodomethane (0.34 mL, 5.46 mmol) was added to a mixture of 57 (746 mg, 2.7 mmol) and potassium carbonate (1.225 g, 8.86 mmol) in DMSO (5 mL). The resulting mixture was stirred at room temperature for one day. Water (50 mL) was added to quench the reaction. The mixture was extracted with ethyl acetate (3×40 mL). The organic solution was washed with brine (2×50 mL), dried over anhydrous sodium sulfate and concentrated to afford a residue. The residue was purified by column chromatography on silica gel (25 g) using ethyl acetate in hexanes (0-20%) to afford 58 (350 mg) as a white solid in 45% yield and 59 (298 mg) as white solid in 38% yield. The product 58 was recrystallized with ethyl acetate and hexanes to afford crystals for X-ray crystallography: mp 107-108° C. IR (KBr) 1694, 1398, 1368, 1297, 1267, 1218, 1007, 966, 871, 850, 831, 752, 705, 649, 595, 561, 549, 483 cm⁻¹; ¹H NMR (300 MHz, CDCl₃) δ 7.91 (s, 1H), 7.61 (s, 1H), 3.63 (s, 3H); ¹³C NMR (75 MHz, CDCl₃) δ 161.1, 158.2, 142.0, 130.2, 122.7, 118.2, 86.1, 32.6, 13.7; ESIMS m/z (rel intensity) 289.94 (MH⁺, 100). Anal. (C₉H₈INO₂) C, H, I, N.

5-Iodo-3-methoxy-7-methylbenzo[d]isoxazole (59): mp 79° C. IR (KBr) 2921, 1603, 1546, 1481, 1453, 1422, 1407, 1366, 1311, 1260, 1228, 1205, 1038, 986, 951, 908, 866, 760 cm⁻¹; ¹H NMR (300 MHz, CDCl₃) δ 7.76 (s, 1H), 7.56 (s, 1H), 4.13 (s, 3H); ¹³C NMR (75 MHz, CDCl₃) δ 166.1, 162.8, 139.1, 127.0, 123.2, 115.9, 85.7, 57.5, 14.3; ESIMS m/z (rel intensity) 289.94 (MH⁺, 24). Anal. (C₉H₈INO₂) C, H, I, N.

Determination of the Structure of 5-Iodo-2,7-dimethyl-benzo[d]isoxazol-3-one (58) by X-ray Crystallography. DATA COLLECTION: A colorless needle of C₉H₈INO₂ having approximate dimensions of 0.50×0.19×0.10 mm was mounted on a glass fiber in a random orientation. Preliminary examination and data collection were performed Mo K_{□} radiation (λ=0.71073 Å) on a Nonius KappaCCD equipped with a graphite crystal, incident beam monochromator. Cell constants for data collection were obtained from least-squares refinement, using the setting angles of 6370 reflections in the range 2<θ<27°. The monoclinic cell parameters and calculated volume are: a=4.1515(5), b=16.0162(12); c=14.1406(11) Å, □=97.411(5)°, V=932.37(15) Å³. For Z=4 and F.W.=289.07 the calculated density is 2.06 g/cm³. The refined mosaicity from DENZO/SCALEPACK was 0.70° indicating moderate crystal quality. The space group was determined by the program ABSEN. See, McArdle, P. “ABSEN-a PC Computer Program for Listing Systematic Absences and Apace-Group Determination” J. Appl. Cryst., 1996, 29(3), 306, the disclosure of which is incorporated herein by reference. From the systematic presences of: h011=2n; 0k0 k=2n, and from subsequent least-squares refinement, the space group was determined to be P2₁/c(# 14). The data were collected at a temperature of 150(1)K. Data were collected to a maximum 2θ of 55.7°.

DATA REDUCTION: A total of 6370 reflections were collected, of which 2194 were unique. Lorentz and polarization corrections were applied to the data. The linear absorption coefficient is 33.6/cm for Mo K_{□} radiation. An empirical absorption correction using SCALEPACK was applied. See, Otwinowski, Z.; Minor, W. “Processing of X-Ray Diffraction Data Collected in Oscillation Mode” Methods Enzymol., 1997, 276, 307-326, the disclosure of which is incorporated herein by reference. Transmission coefficients ranged from 0.623 to 0.715. Intensities of equivalent reflections were averaged. The agreement factor for the averaging was 3.7% based on intensity.

STRUCTURE SOLUTION AND REFINEMENT: The structure was solved using the structure solution program PATTY in DIRDIF99. See, Burla, M. C.; Camalli, M.; Carrozzini, B.; Cascarano, G. L.; Giacovazzo, C.; Polidori, G.; Spagna, R. SIR2002: the Program J. Appl. Cryst., 2003, 36, 1103, the disclosure of which is incorporated herein by reference. The remaining atoms were located in succeeding difference Fourier syntheses. Hydrogen atoms were included in the refinement but restrained to ride on the atom to which they are bonded. The structure was refined in full-matrix least-squares where the function minimized was Σw(|Fo|²−|Fc|²)² and the weight w is defined as 1/[σ²(Fo²)+(0.0384P)²+0.751 P] where P=(Fo²+2Fc²)/3. Scattering factors were taken from the “International Tables for Crystallography”. See, “International Tables for Crystallography”, Vol. C, Kluwer Academic Publishers, Dordrecht, The Netherlands, 1992, Tables 4.2.6.8 and 6.1.1.4, the disclosure of which is incorporated herein by reference. 2194 reflections were used in the refinements. However, only the 1876 reflections with Fₒ²>2σ(Fₒ²) were used in, calculating R1. The final cycle of refinement included 120 variable parameters and converged (largest parameter shift was <0.01 times its su) with unweighted and weighted agreement factors of:

R1=Σ|Fo−Fc|√ΣFo=0.029

R2=SQRT(Σw(Fo²−Fc²)²/Σw(Fo²)²)=0.071

The standard deviation of an observation of unit weight was 1.07. The highest peak in the final difference Fourier had a height of 0.62 e/A³. The minimum negative peak had a height of −1.58 e/A³. Refinement was performed on a LINUX PC using SHELX-97. G. M. Sheldrick, SHELXL97. A Program for Crystal Structure Refinement. Univ. of Gottingen, Germany, 1997, the disclosure of which is incorporated herein by reference. Crystallographic drawings were done using programs ORTEP (See, C. K. Johnson, ORTEPII, Report ORNL-5138, Oak Ridge National Laboratory, Tennessee, USA, 1976, the disclosure of which is incorporated herein by reference.) and PLUTON (See, A. L. Spek, PLUTON. Molecular Graphics Program. Univ. of Ultrecht, The Netherlands, 1991, the disclosure of which is incorporated herein by reference.).

(Z)-5-[1-(3,7-Dimethyl-2-oxo-2,3-dihydro-benzoxazol-5-yl)-5-methoxycarbonyl-pent-1-enyl]-2-methoxy-3-methylbenzoic Acid Methyl Ester (60). The general procedure was followed using vinylstannane 72 (381 mg, 0.658 mmol), 5-iodo-2-methoxy-3-methylbenzoic acid methyl ester (29) (245 mg, 0.800 mmol), cesium fluoride (395 mg, 2.57 mmol) and Pd(PBu^{t}₃)₂ (36 mg, 0.069 mmol) in toluene (1 mL). The mixture was stirred under argon at room temperature for 22 h, at 60° C. for 31.5 h, and at 110° C. for 13.5 h. The residue was purified by column chromatography on silica gel (20 g), eluting with EtOAc-hexanes (0-30%) to afford the product 60 (202 mg) as an oil in 66% yield. IR (KBr) 2950, 1780, 1732, 1618, 1475, 1436, 1352, 1232, 1196, 1137, 1062, 1007, 880, 750, 621 cm⁻¹; ¹H NMR δ 7.38 (d, J=2.4 Hz, 1H), 7.05 (d, J=2.4 Hz, 1H), 6.64 (s, 1H), 6.51 (s, 1H), 5.93 (t, J=7.5 Hz, 1H), 3.78 (s, 3H), 3.72 (s, 3H), 3.53 (s, 3H), 3.30 (s, 3H), 2.29 (s, 3H), 2.24 (t, J=7.5 Hz, 2H), 2.17 (s, 3H), 2.05 (q, J=7.5 Hz, 2H), 1.70 (dt, J=7.5 Hz, 2H); ¹³C NMR (75 MHz, CDCl₃) δ 173.48, 166.71, 157.11, 154.70, 140.57, 140.11, 137.67, 135.05, 133.36, 132.12, 131.27, 129.17, 127.17, 125.30, 124.10, 119.92, 106.71, 61.22, 51.93, 51.18, 33.14, 28.95, 27.98, 24.69, 15.86, 14.24; ESIMS m/z (rel intensity) 467.77 (MH⁺, 30), 436.18 (M-OCH₃⁺, 100). Anal. Calcd for (C₂₆H₂₉NO₇) C, H, N.

(E)-3-Chloro-5-[1-(3,7-dimethyl-2-oxo-2,3-dihydro-benzoxazol-5-yl)-5-methoxycarbonyl-pent-1-enyl]-2-methoxybenzoic Acid Methyl Ester (61). The general procedure was followed using vinylstannane 72 (352 mg, 0.608 mmol), aryl iodide 55 (226 mg, 0.815 mmol), cesium fluoride (350 mg, 2.28 mmol) and Pd(PBu^{t}₃)₂ (34 mg, 0.064 mmol) in toluene (1 mL). The mixture was stirred under argon at room temperature for 17.5 h, at 60° C. for 24.5 h, and then at 110° C. for 24 h. The residue was purified by column chromatography on silica gel (15 g), eluting with EtOAc-hexanes (0-30%) to afford the product 61 (54.7 mg) as an oil in 18% yield and 62 (35 mg) as solid in 13% yield. Spectra of 61: ¹H NMR δ 7.48 (d, J=1.8 Hz, 1H), 7.26 (d, J=2.1 Hz, 1H), 6.66 (s, 1H), 6.52 (s, 1H), 6.00 (t, J=7.2 Hz, 1H), 3.88 (s, 3H), 3.86 (s, 3H), 3.58 (s, 3H), 2.34 (s, 3H), 2.26 (t, J=7.2 Hz, 2H), 2.09 (q, J=7.5 Hz, 2H), 1.74 (dt, J=7.5 Hz, 2H); ESIMS m/z (rel intensity) 487.67/489.73 (MH⁺, 57/25). Anal. Calcd for (C₂₅H₂₆ClNO₇) C, H, Cl, N.

(Z)-5-[1-(3,7-Dimethyl-2-oxo-2,3-dihydrobenzoxazol-5-yl)-5-methoxycarbonyl-pent-1-enyl]-2-methoxy-benzoic Acid Methyl Ester (62). Compound 62 was obtained as described above: mp 134-135° C. IR (KBr) 2949, 1778, 1732, 1618, 1499, 1463, 1436, 1353, 1306, 1266, 1192, 1157, 1083, 1028, 750 cm⁻¹; ¹H NMR δ 7.62 (d, J=2.4 Hz, 1H), 7.19 (dd, J=2.4 Hz and 8.7 Hz, 1H), 6.83 (d, J=8.7 Hz, 1H), 6.69 (s, 1H), 6.53 (s, 1H), 5.96 (t, J=7.5 Hz, 1H), 3.85 (s, 3H), 3.84 (s, 3H), 3.59 (s, 3H), 3.35 (s, 3H), 2.35 (s, 3H), 2.27 (t, J=7.5 Hz, 2H), 2.10 (q, J=7.5 Hz, 2H), 1.75 (dt, J=7.5 Hz, 2H); ¹³C NMR δ 173.76, 166.78, 158.08, 154.97, 140.54, 140.31, 135.39, 134.70, 132.08, 131.43, 129.85, 128.43, 125.57, 120.20, 119.87, 111.69, 106.86, 56.07, 52.08, 51.44, 33.40, 29.16, 28.18, 24.95, 14.47; ESIMS m/z (rel intensity) 453.78 (MH⁺, 21), 476.02 (MNa⁺, 17). Anal. Calcd for (C₂₅H₂₇NO₇) C, H, N.

(Z)-6-(3,7-Dimethyl-2-oxo-2,3-dihydrobenzoxazol-5-yl)-6-(3-fluoro-5-trifluoromethylphenyl)-hex-5-enoic Acid Methyl Ester (63). The general procedure was followed using vinylstannane 43 (325 mg, 0.561 mmol), aryl iodide 70 (245 mg, 0.847 mmol), cesium fluoride (275 mg, 1.792 mmol), and Pd(PBu^{t}₃)₂ (30 mg, 0.053 mmol) in toluene (1 mL). The mixture was stirred under argon at room temperature for 19 h, at 60° C. for 24 h, and then at 110° C. for 9 h. The residue was purified by column chromatography on silica gel (25 g), eluting with EtOAc-hexanes (0-5%) to afford the product 63 (98 mg) as an oil in 39% yield. IR (KBr) 2952, 1778, 1737, 1560, 1438, 1316, 1214, 1169, 1129, 936, 878, 750 cm⁻¹; ¹H NMR δ 7.29 (d, J=7.8 Hz, 2H), 7.20 (s, 1H), 7.05 (d, J=8.4 Hz, 1H), 6.68 (s, 1H), 6.54 (s, 1H), 6.03 (t, J=7.5 Hz, 1H), 3.62 (s, 3H), 3.33 (s, 3H), 2.31 (s, 3H), 2.30 (t, J=7.5 Hz, 2H), 2.16-2.08 (q, J=7.2-7.8 Hz, 2H), 1.84-1.74 (m, 2H); ¹³C NMR δ 173.57, 163.98, 160.67, 154.89, 143.21, 140.76, 139.89, 137.63, 131.49, 130.88, 123.57, 122.32, 120.37, 120.17, 111.88, 104.40, 51.48, 33.30, 29.05, 28.16, 24.78, 14.39; ESIMS m/z (rel intensity) 452.26 (MH⁺, 67). Anal. Calcd for (C₂₃H₂₁F₄NO₄) C, H, F, N.

(E)-5-[1-(3,7-Dimethyl-2-oxo-2,3-dihydro-benzoxazol-5-yl)-5-methoxycarbonyl-pent-1-enyl]-2-methoxy-3-methylbenzoic Acid Methyl Ester (64). The general procedure was followed using vinylstannane 52 (342 mg, 0.574 mmol), aryl iodide 70 (227 mg, 0.785 mmol), cesium fluoride (315 mg, 2.05 mmol), and Pd(PBu^{t}₃)₂ (34 mg, 0.065 mmol) in toluene (1 mL). The mixture was stirred under argon at room temperature for 10 h, at 50° C. for 24 h, and then at 100° C. for 14.5 h. The residue was purified by column chromatography on silica gel (25 g), eluting with EtOAc-hexanes (0-30%) to afford the product 64 (195 mg) as a white solid in 73% yield: mp 120-120.5° C. IR (KBr) 2950, 1732, 1548, 1496, 1436, 1395, 1318, 1255, 1203, 1141, 1009, 910, 765 cm⁻¹; ¹H NMR δ 7.36 (d, J=2.1 Hz, 1H), 7.05 (d, J=2.0 Hz, 1H), 6.68 (s, 1H), 6.53 (d, J=1.5 Hz, 1H), 5.89 (t, J=7.5 Hz, 1H), 3.84 (s, 3H), 3.81 (s, 3H), 3.56 (s, 3H), 3.27 (s, 3H), 2.256 (s, 3H), 2.255 (t, J=7-0.5 Hz, 3H), 2.247 (s, 3H), 2.13-2.05 (q, J=7.4 Hz, 2H), 1.77-1.68 (m, 2H); ¹³C NMR δ 173.66, 166.61, 157.28, 154.85, 140.78, 140.35, 138.67, 136.14, 134.90, 132.61, 131.13, 130.11, 129.43, 124.18, 123.45, 119.70, 104.48, 61.36, 52.05, 51.32, 33.30, 29.00, 28.05, 16.01, 14.29; ESIMS m/z (rel intensity) 467.97 (MH⁺, 95). Anal. Calcd for (C₂₆H₂₉NO₇) C, H, N.

(Z)-6-(2,7-Dimethyl-3-oxo-2,3-dihydro-benzo[d]isoxazol-5-yl)-6-(3,7-dimethyl-2-oxo-2,3-dihydro-benzoxazol-5-yl)-hex-5-enoic Acid Methyl Ester (65). The general procedure was followed using vinylstannane 72 (370 mg, 0.640 mmol), 5-iodo-2,7-dimethyl-benzo[d]isoxazol-3-one (58) (231 mg, 0.80 mmol), cesium fluoride (404 mg, 2.63 mmol) and Pd(PBu^{t}₃)₂ (42 mg, 0.08 mmol) in toluene (1 mL). The mixture was stirred under argon at room temperature for 22.5 h, at 70° C. for 24.5 h, and then at 110° C. for 27 h. The residue was purified by column chromatography on silica gel (20 g), eluting with EtOAc-hexanes (0-50%) to afford the product 65 (101 mg) as a solid in 35% yield: mp 152.5-154° C. IR (KBr) 2949, 1778, 1736, 1690, 1617, 1492, 1470, 1355, 1298, 1249, 1224, 1169, 1062, 1032, 880, 854, 771, 750, 619 cm⁻¹; ¹H NMR δ 7.27 (s, 1H), 7.19 (s, 1H), 6.60 (s, 1H), 6.45 (s, 1H), 5.92 (t, J=7.5 Hz, 1H), 3.55 (s, 3H), 3.51 (s, 3H), 3.26 (s, 3H), 2.26 (s, 3H), 2.24 (s, 3H), 2.20 (t, J=7.5 Hz, 2H), 2.03 (q, J=7.5 Hz, 2H), 1.67 (dt, J=7.5 Hz, 2H); ¹³C NMR δ 173.64, 162.97, 158.02, 154.87, 140.83, 140.34, 138.59, 135.28, 132.80, 131.46, 129.47, 125.48, 120.27, 119.80, 115.74, 106.79, 51.38, 33.29, 32.54, 29.13, 28.11, 24.82, 14.40, 14.05; ESIMS m/z (rel intensity) 473.03 (MNa⁺, 100). Anal. Calcd for (C₂₅H₂₆N₂O₆) C, H, N.

6,6-Bis-(3,7-dimethyl-2-oxo-2,3-dihydrobenzoxazol-5-yl)-hex-5-enoic Acid Methyl Ester (66). The general procedure was followed using vinylstannane 72 (371 mg, 0.642 mmol), 5-iodo-3,7-dimethyl-3H-benzoxazol-2-one (70) (226 mg, 0.782 mmol), cesium fluoride (334 mg, 2.18 mmol) and Pd(PBu^{t}₃)₂ (35 mg, 0.067 mmol) in toluene (1 mL). The mixture was stirred under argon at room temperature for 22.5 h, at 60° C. for 24.5 h, and then at 110° C. for 27 h. The residue was purified by column chromatography on silica gel (15 g), eluting with EtOAc-hexanes (0-50%) to afford the product 66 (133 mg) as a solid in 46% yield: mp 163.5-165° C. IR (KBr) 2948, 1772, 1734, 1638, 1618, 1494, 1469, 1358, 1302, 1165, 1064, 880, 749, 625 cm⁻¹; ¹H NMR δ 6.70 (s, 1H), 6.68 (s, 1H), 6.56 (d, J=1.5 Hz, 1H), 6.54 (d, J=0.9 Hz, 1H), 5.94 (t, J=7.5 Hz, 1H), 3.57 (s, 3H), 3.33 (s, 3H), 3.27 (s, 3H), 2.33 (s, 3H), 2.27 (t, J=7.5 Hz, 2H), 2.25 (s, 3H), 2.09 (q, J=7.5 Hz, 2H), 1.74 (dt, J=7.5 Hz, 2H); ¹³C NMR δ 173.64, 154.85, 141.45, 140.38, 140.23, 138.83, 135.48, 131.36, 131.18, 129.26, 125.45, 123.37, 120.07, 119.72, 106.81, 104.40, 51.35, 33.30, 29.13, 28.07, 24.84, 20.87, 14.37, 14.31; ESIMS m/z (rel intensity) 450.96 (MH⁺, 100). Anal. Calcd for (C₂₅H₂₆N₂O₆) C, H, N.

(Z)-6-(3,7-Dimethyl-2-oxo-2,3-dihydro-benzoxazol-5-yl)-6-(3-methoxy-7-methyl-benzo[d]isoxazol-5-yl)-hex-5-enoic Acid Methyl Ester (67). The general procedure was followed using vinylstannane 72 (374 mg, 0.647 mmol), 5-iodo-3-methoxy-7-methylbenzo[d]isoxazole (59) (229 mg, 0.792 mmol), cesium fluoride (355 mg, 2.31 mmol) and Pd(PBu^{t}₃)₂ (36 mg, 0.069 mmol) in toluene (1 mL). The mixture was stirred under argon at room temperature for 22 h, at 60° C. for 31 h, and then at 110° C. for 16.5 h. The residue was purified by column chromatography on silica gel (20 g), eluting with EtOAc-hexanes (0-65%) to afford the product 67 (114 mg) as solid in 46% yield: mp 54-55° C. IR (KBr) 2947, 1778, 1736, 1617, 1548, 1495, 1458, 1357, 1297, 1208, 1167, 1062, 878, 750, 690, 619 cm⁻¹; ¹H NMR δ 7.17 (s, 1H), 7.12 (s, 1H), 6.69 (s, 1H), 6.54 (s, 1H), 5.97 (t, J=7.5 Hz, 1H), 4.06 (s, 3H), 3.55 (s, 3H), 3.32 (s, 3H), 2.39 (s, 3H), 2.33 (s, 3H), 2.27 (t, J=7.5 Hz, 2H), 2.10 (q, J=7.5 Hz, 2H), 1.74 (dt, J=7.5 Hz, 2H); ¹³C NMR δ 173.62, 167.19, 162.51, 154.82, 141.17, 140.23, 138.42, 135.56, 131.39, 130.18, 129.20, 125.45, 120.25, 120.10, 116.35, 113.44, 106.81, 57.14, 51.33, 33.27, 29.13, 28.07, 24.84, 14.51, 14.36; ESIMS m/z (rel intensity) 451.13 (MH⁺, 38), 473.02 (MNa⁺, 45). Anal. Calcd for (C₂₅H₂₆N₂O₆) C, H, N.

(E)-5-[1-(3-Methoxy-7-methylbenzo[d]isoxazol-5-yl)-4-(2-oxoxazolidin-3-yl)-but-1-enyl]-3,7-dimethyl-3H-benzoxazol-2-one (68). The general procedure was followed using vinylstannane 74 (390 mg, 0.659 mmol), 5-iodo-3,7-dimethyl-3H-benzoxazol-2-one (70) (229 mg, 0.791 mmol), cesium fluoride (355 mg, 2.31 mmol) and Pd(PBu^{t}₃)₂ (35 mg, 0.067 mmol) in toluene (1 mL). The mixture was stirred under argon at room temperature for 21 h, at 60° C. for 24 h, and then at 110° C. for 25 h. The residue was purified by column chromatography on silica gel (20 g), eluting with EtOAc-hexanes (0-50%) to afford the product 68 (140 mg) as solid in 46% yield: mp 187-188° C. IR (KBr) 2925, 1775, 1618, 1548, 1496, 1448, 1426, 1359, 1333, 1305, 1267, 1226, 1153, 1104, 1065, 1043, 973, 955, 912, 881 cm⁻¹; ¹H NMR δ 7.23 (s, 1H), 7.05 (s, 1H), 6.67 (s, 1H), 6.64 (s, 1H), 5.97 (t, J=7.5 Hz, 1H), 4.21 (t, J=7.8 Hz, 2H), 4.14 (s, 3H), 3.37 (t, J=6.6 Hz, 2H), 3.32 (s, 3H), 3.25 (t, J=8.1 Hz, 2H), 2.48 (s, 3H), 2.39 (t, J=6.6 Hz, 2H), 2.28 (s, 3H); ¹³C NMR δ 167.19, 162.52, 158.49, 154.99, 142.91, 140.63, 138.68, 134.99, 132.50, 131.31, 126.14, 123.80, 121.09, 119.87, 118.79, 113.68, 104.88, 61.52, 57.37, 44.10, 43.79, 28.15, 27.74, 14.64, 14.40; ESIMS m/z (rel intensity) 486.00 (MNa⁺, 100); negative ion ESIMS m/z (rel intensity) 432.36 [(M−H⁺)⁻, 100]. Anal. Calcd for (C₂₅H₂₅N₃O₆) C, H, N.

5-Iodo-7-methyl-3H-benzoxazol-2-one (69). Diethyl azodicarboxylate (1.27 mL, 7.90 mmol) was added dropwise to a solution of 2,N-dihydroxy-5-iodo-3-methylbenzamide (56) (1.55 g, 5.28 mmol) and PPh₃ (2.09 g, 7.90 mmol) in THF (50 mL) at 0° C. The reaction mixture was stirred at 0° C. for 45 min and at room temperature for 2.5 h. The mixture was quenched with a 1:1 mixture of methanol-acetic acid (2.0 mL) and then concentrated. The residue was purified by column chromatography on silica gel (30 g) using EtOAc-hexanes (0-10%) to afford the product 69 (879 mg) as white crystals in 60% yield: mp 248-250° C. IR (KBr) 3153, 3040, 2977, 2922, 1865, 1790, 1722, 1640, 1615, 1480, 1438, 1385, 1370, 1319, 1296, 1158, 1099, 1067, 943, 932, 836, 736 cm⁻¹; ¹H NMR (acetone-d₆) δ 7.31 (s, 1H), 7.28 (s, 1H), 2.81 (s, 1H), 2.30 (s, 3H); negative ion ESIMS m/z (rel intensity) 274.18 [(M−H⁺)⁻, 100]. Anal. Calcd for (C₈H₆₁NO₂) C, H, I, N.

5-Iodo-3,7-dimethyl-3H-benzoxazol-2-one (70). Method I: Compound 69 (105 mg, 0.38 mmol) was dissolved in ethyl acetate (8 mL), and then tetrabutylammonium iodide (15 mg, 0.04 mmol) was added, followed by addition of a solution of potassium carbonate (193 mg, 1.40 mmol) in water (3 mL). Iodomethane (0.06 mL, 0.9637 mmol) was added dropwise. The resulting mixture was stirred at room temperature for one day. The organic phase was separated and the aqueous phase was extracted with ethyl acetate (2×15 mL). The combined organic solution was washed with brine (2×50 mL), dried over anhydrous sodium sulfate, and concentrated to afford a residue. The residue was purified by column chromatography on silica gel (15 g) using ethyl acetate in hexanes (0-10%) to afford the product 70 (92 mg) in 84%.

Method II: Iodomethane (0.05 mL, 0.795 mmol) was added to a mixture of compound 69 (98 mg, 0.35 mmol) and potassium carbonate (148 mg, 1.07 mmol) in DMSO (5 mL). The resulting mixture was stirred at room temperature for one day. Water (10 mL) was added to quench the reaction. The mixture was extracted with ethyl acetate (3×15 mL). The organic solution was washed with brine (2×50 mL), dried over anhydrous sodium sulfate, and concentrated to afford a residue. The residue was purified by column chromatography on silica gel using ethyl acetate in hexanes to afford the product 70 (84 mg) as a white solid in 82% yield. The product was recrystallized from ethyl acetate and hexanes to afford crystals for X-ray crystallography: mp 100-101° C. IR (film) 2924, 1775, 1639, 1602, 1486, 1360, 1292, 1247, 1210, 1176, 1060, 1026, 882, 838, 746, 624, 666 cm⁻; ¹H NMR δ 7.28 (s, 1H), 7.09 (s, 1H), 3.34 (s, 3H), 2.31 (s, 3H); ¹³C NMR δ 154.10, 140.86, 132.75, 132.56, 122.58, 114.33, 85.87, 28.24, 14.06; ESIMS m/z (rel intensity) 290.07 (MH⁺, 100). Anal. Calcd for (C₉H₈₁NO₂) C, H, I, N.

Determination of the Structure of 5-Iodo-3,7-dimethyl-3H-benzoxazol-2-one (70) by X-ray Crystallography.

DATA COLLECTION: A colorless plate of C₉H₈INO₂ having approximate dimensions of 0.30×0.30×0.10 mm was mounted on a glass fiber in a random orientation. Preliminary examination and data collection were performed by Mo K_{α} radiation (λ=0.71073 Å) on a Nonius KappaCCD equipped with a graphite crystal, incident beam monochromator. Cell constants for data collection were obtained from least-squares refinement, using the setting angles of 46585 reflections in the range 2<θ<26°. The orthorhombic cell parameters and calculated volume are: a=13.7861(4), b=16.5709(4), c=34.1035(10)Å, V=7790.9(4) Å³. For Z=32 and F.W.=289.07 the calculated density is 1.97 g/cm³. The refined mosaicity from DENZO/SCALEPACK was 0.74° indicating moderate crystal quality. The space group was determined by the program ABSEN. See, McArdle, P. ABSEN—A PC Computer Program for Listing Systematic Absences and Space-group Determination. J. Appl. Crystallogr. 1966, 29, 306, the disclosure of which is incorporated herein by reference. From the systematic presences of: hk0 h=2n; h01 1=2n; 0k1 k=2n; and from subsequent least-squares refinement, the space group was determined to be Pbca (#61). The data were collected at a temperature of 150(1) K. Data were collected to a maximum 20 of 53.4°.

DATA REDUCTION: A total of 46585 reflections were collected, of which 8255 were unique. Lorentz and polarization corrections were applied to the data. The linear absorption coefficient is 32.2/cm for Mo K_{α} radiation. An empirical absorption correction using SCALEPACK was applied. See, Otwinowski, Z.; Minor, Z. Processing of X-Ray Diffraction Data Collected in Oscillation Mode. Methods Enzymol. 1997, 276, 307-326, the disclosure of which is incorporated herein by reference. Transmission coefficients ranged from 0.589 to 0.725. Intensities of equivalent reflections were averaged. The agreement factor for the averaging was 0.8% based on intensity.

STRUCTURE SOLUTION AND REFINEMENT: The structure was solved by direct methods using SIR2002. See, Burla, M. C.; Camalli, M.; Carrozzini, B.; Cascarano, G. L.; Giacovazzo, C.; Polidori, G.; Spagna, R. SIR2002: the Program. J. Appl. Cryst. 2003, 36, 1103, the disclosure of which is incorporated herein by reference. The remaining atoms were located in succeeding difference Fourier syntheses. Hydrogen atoms were included in the refinement but restrained to ride on the atom to which they are bonded. The structure was refined in full-matrix least-squares where the function minimized was Σw(|Fo|²−|Fc|²)² and the weight w is defined as 1/[σ²(Fo²)+(0.0365P)²+0.0000P] where P=(Fo²+2Fc²)/3. Scattering factors were taken from the “International Tables for Crystallography” and 8255 reflections were used in the refinements. See, International Tables for Crystallography, Volume C. Mathematical, Physical and Chemical Tables.; Kluwer Academic Publishers Dordrecht, The Netherlands, 1992; Tables 4.2.6.8 and 6.1.1.4, the disclosure of which is incorporated herein by reference. However, only the 4203 reflections with Fₒ²>2σ (Fₒ²) were used in calculating R1. The final cycle of refinement included 596 variable parameters and converged (largest parameter shift was <0.01 times its su) with unweighted and weighted agreement factors of:

R1=Σ|Fo−Fc|/ΣFo=0.037

R2=SQRT(Σw(Fo²−Fc²)₂/Σw(Fo²)²)=0.071

The standard deviation of an observation of unit weight was 0.84. The highest peak in the final difference Fourier had a height of 0.70 e/A³. The minimum negative peak had a height of −0.98 e/A³. Refinement was performed on a LINUX PC using SHELX-97. See, Sheldrick, G. M. SHELEX97, Program for Crystal Structure Refinement; University of Gottingen: Germany, 1997, the disclosure of which is incorporated herein by reference. Crystallographic drawings were done using programs ORTEP and PLUTON. See, Johnson, C. K. OrtepII Report ORNL-5138; Oak Ridge National Laboratory: Tennessee, U.S.A., 1976; Spek, A. L. PLUTON. Molecular Graphics Program; Univ. of Ultrecht: Ultrecht, The Netherlands, 1991, the disclosures of which are incorporated herein by reference.

6-(3,7-Dimethyl-2-oxo-2,3-dihydrobenzoxazol-5-yl)-hex-5-ynoic Acid Methyl Ester (71). 5-Iodo-3,7-dimethyl-3H-benzoxazol-2-one (70) (2.335 g, 8.077 mmol) and hex-5-ynoic acid methyl ester (1.019 g, 8.074 mmol) were dissolved in THF (15 mL) at room temperature. Triethylamine (3.0 mL, 21.52 mmol), Pd(PPh₃)Cl₂ (285 mg, 0.4 mmol) and Cu(I)I (154 mg, 8.076 mmol) were added. After the resulting mixture was stirred at room temperature for 23 h, water (50 mL) was added to quench the reaction. The mixture was concentrated to remove the organic solvents, and the residue was extracted with ethyl acetate (3×50 mL). The combined organic solution was washed with brine (100 mL), dried over Na₂SO₄, and concentrated. The residue was purified by column chromatography on silica gel (40 g), eluting with EtOAc-hexanes (0-30%) to afford the product 71 (1.729 g) as white solid in 75% yield: mp 97-98° C. IR (KBr) 2950, 1779, 1735, 1619, 1468, 1374, 1332, 1302, 1211, 1158, 1061, 880, 749, 681, 630 cm⁻¹; ¹H NMR δ 6.99 (s, 1H), 6.80 (s, 1H), 3.67 (s, 3H), 3.35 (s, 3H), 2.49 (t, J=7.5 Hz, 2H), 2.46 (t, J=6.9 Hz, 2H), 2.31 (s, 3H), 1.91 (m, 2H); ¹³C NMR δ 173.45, 154.64, 140.60, 131.12, 127.81, 120.27, 119.04, 108.48, 88.16, 80.70, 51.54, 32.79, 28.08, 23.76, 18.80, 14.14; ESIMS m/z (rel intensity) 309.82 (MNa⁺, 5), 596.62 (2M+Na⁺, 100). Anal. Calcd for (C₁₆H₁₇NO₄) C, H, N.

6-(3,7-Dimethyl-2-oxo-2,3-dihydro-benzoxazol-5-yl)-6-(tributylstannanyl)-hex-5-enoic Acid Methyl Ester (72). 6-(3,7-Dimethyl-2-oxo-2,3-dihydrobenzoxazol-5-yl)-hex-5-ynoic acid methyl ester (71) (1.70 g, 5.92 mmol) was dissolved in THF (310 mL), and then tetrakis(triphenylphosphine)palladium (68 mg, 0.059 mmol) was added. The mixture was cooled to 0° C., degassed by gently bubbling argon for 20 min, and then tributyltin hydride (2.5 mL, 9.02 mmol) was added dropwise over 30 min. The mixture was stirred at 0° C. for 60 min and at room temperature for 5 h, and then concentrated to yield a residue. The residue was purified by column chromatography on silica gel (60 g), using hexanes and EtOAc-hexanes (5%) to afford the vinylstannane 72 (3.07 g) as oil in 90% yield. IR (KBr) 2954, 2926, 2851, 1781, 1740, 1616, 1491, 1459, 1374, 1294, 1206, 1158, 1064, 1027, 879, 751, 686 cm⁻¹; ¹H NMR δ 6.42 (s, 1H), 6.29 (s, 1H), 5.70 (t, J=6.9 Hz, 1H), 3.58 (s, 3H), 3.34 (s, 3H), 2.31 (s, 3H), 2.23 (t, J=7.5 Hz, 2H), 3.02 (q, J=7.2-7.5 Hz, 2H), 1.71-1.63 (m, 2H), 1.49-1.36 (m, 6H), 1.30-1.18 (m, 6H), 0.86-0.78 (m, 9H); ¹³C NMR δ 173.92, 155.00, 145.98, 141.17, 140.87, 138.83, 131.16, 122.43, 119.90, 103.82, 51.43, 33.40, 29.36, 28.97, 28.07, 27.28, 24.84, 14.51, 13.67, 9.90; ESIMS m/z (rel intensity) 600.23 (MNa⁺, 22), 602.08 (MNa⁺, 20). Anal. Calcd for (C₂₈H₄₅NO₄Sn) C, H, N, Sn.

3-[4-(3-Methoxy-7-methylbenzo[d]isoxazol-5-yl)-but-3-ynyl]-oxazolidin-2-one (73). 5-Iodo-3-methoxy-7-methyl-benzo[d]isoxazole (59) (3.405 g, 11.78 mmol) and 3-but-3-ynyl-1,3-oxazolidine-2-one (25) (1.485 g, 10.68 mmol) were dissolved in THF (25 mL) at room temperature. Triethylamine (3.8 mL, 27 mmol), Pd(PPh₃)Cl₂ (386 mg, 0.539 mmol) and Cu(I)I (207 mg, 1.06 mmol) were added. After the resulting mixture was stirred at room temperature for 26 h, water (40 mL) was added to quench the reaction. The mixture was concentrated to remove the organic solvents, and the residue was extracted with ethyl acetate (3×50 mL). The combined organic solution was washed with brine (150 mL), dried over Na₂SO₄, and concentrated. The residue was purified by column chromatography on silica gel (60 g), eluting with EtOAc-hexanes (0-50%) to afford the product 73 (2.816 g) as brown solid in 88% yield: mp 94-95° C. IR (KBr) 2943, 2238, 1751, 1613, 1549, 1497, 1426, 1390, 1312, 1268, 1223, 1091, 1042, 971, 909, 763, 693 cm⁻¹; ¹H NMR δ 7.45 (s, 1H), 7.30 (s, 1H), 4.34 (t, J=8.0 Hz, 2H), 4.13 (s, 3H), 3.74 (t, J=8.0 Hz, 2H), 3.52 (t, J=6.6 Hz, 2H), 2.68 (t, J=6.6 Hz, 2H), 2.44 (s, 3H); ¹³C NMR δ 166.92, 162.54, 158.27, 133.99, 121.36, 121.04, 118.33, 113.67, 85.64, 81.46, 61.82, 57.35, 45.03, 43.18, 18.85, 14.37; ESIMS m/z (rel intensity) 300.93 (MH⁺, 44), 600.72 (2M+H⁺, 100). Anal. Calcd for (C₁₆H₁₆N₂O₄) C, H, N.

3-[4-(3-Methoxy-7-methylbenzo[d]isoxazol-5-yl)-4-(tributylstannanyl)-but-3-enyl]-oxazolidin-2-one (74). The alkyne 73 (2.746 g, 9.14 mmol) was dissolved in THF (420 mL), and then tetrakis(triphenylphosphine)palladium (108 mg, 0.092 mmol) was added. The mixture was cooled to 0° C., degassed by gently bubbling argon for 20 min, and then tributyltin hydride (3.8 mL, 13.7 mmol) was added dropwise over 90 min. The mixture was stirred at 0° C. for 30 min and at room temperature for 160 min, and then concentrated to yield a residue. The residue was purified by column chromatography on silica gel (100 g), using hexanes and EtOAc-hexanes (0-30%) to afford the product 74 (4.287 g) as oil in 79% yield and 75 (445 mg) as oil in 8% yield. Spectral data of 74: IR (KBr) 2952, 2925, 2851, 1756, 1546, 1492, 1224, 1358, 1305, 1265, 1216, 1173, 1097, 1043, 961, 910, 878, 763, 693 cm⁻¹; ¹H NMR δ 6.90 (s, 1H), 6.84 (s, 1H), 5.75 (t, J=6.9 Hz, 1H), 4.22 (t, J=7.8 Hz, 2H), 4.14 (s, 3H), 3.30 (t, J=7.8 Hz, 2H), 3.27 (t, J=7.2 Hz, 2H), 2.46 (s, 3H), 2.30-2.23 (m, 2H), 1.45-1.35 (m, 6H), 1.32-1.17 (m, 6H), 0.87-0.79 (m, 9H); ¹³C NMR δ 167.23, 158.27, 148.31, 140.14, 137.74, 130.28, 120.53, 114.66, 113.63, 61.51, 57.28, 44.27, 43.84, 28.92, 28.04, 27.24, 14.72, 13.63, 9.92; ESIMS m/z (rel intensity) 610.84 (MNa⁺, 25), 613.06 (MNa⁺, 33), 614.93 (MNa⁺, 40). Anal. Calcd for (C₂₈H₄₄N₂O₄Sn) C, H, N, Sn.

3-[4-(3-Methoxy-7-methylbenzo[d]isoxazol-5-yl)-3-(tributylstannanyl)-but-3-enyl]-oxazolidin-2-one (75). Compound 75 was obtained as described above: IR (KBr) 2956, 2925, 2871, 2853, 1756, 1614, 1548, 1490, 1457, 1424, 1389, 1307, 1273, 1220, 1101, 1046, 961, 912, 807, 764, 697 cm⁻¹; ¹H NMR δ 7.19 (s, 1H), 7.15 (s, 1H), 6.69 (s, 1H), 4.13 (t, J=7.5-8.4 Hz, 2H), 4.07 (s, 3H), 3.31 (t, J=7.8-8.4 Hz, 2H), 3.24 (t, J=7.5-8.1 Hz, 2H), 2.66 (t, J=8.4 Hz, 2H), 2.42 (s, 3H), 1.60-1.42 (m, 6H), 1.36-1.21 (m, 6H), 0.99-0.90 (m, 3H), 0.85 (t, J=7.2 Hz, 6H); NMR δ 167.09, 161.85, 157.93, 144.73, 140.24, 133.28, 131.55, 120.44, 117.01, 113.38, 61.38, 57.09, 44.16, 44.07, 31.65, 28.90, 27.16, 14.36, 13.50, 9.79; ESIMS m/z (rel intensity) 610.98 (MNa⁺, 53), 613.17 (MNa⁺, 89), 615.07 (MNa⁺, 100). Anal. Calcd for (C₂₈H₄₄N₂O₄Sn) C, H, N, Sn.

RT Inhibition Assay. Analysis of the effects of the compounds on recombinant HIV-1 RT enzyme (p66/51 dimer) was performed as previously described. See, Buckheit, R. W. J.; Fliakas-Boltz, V.; Decker, W. D.; Robertson, J. L.; Stup, T. L.; Pyle, C. A.; White, E. L.; McMahon, J. B.; Currens, M. J.; Boyd, M. R.; Bader, J. P. Comparative Anti-HIV Evaluation of Diverse HIV-1-Specific Reverse Transcriptase Inhibitor-Resistant Virus Isolates Demonstrates the Existence of Distinct Phenotypic Subgroups. Antiviral Res. 1995, 26, 117-132, the disclosure of which is incorporated herein by reference. Briefly, inhibition of purified recombinant reverse transcriptase enzyme was measured by the incorporation of [³²P]GTP into poly(rC)/oligo(dG) (rCdG) homopolymer template primers.

In Vitro Antiviral Assays. Evaluation of the antiviral activity of compounds against HIV-1_{RF} infection in CEM-SS cells was performed using the MTS cytoprotection assay as previously described. See, Rice, W. G.; Bader, J. P. Discovery and in Vitro Development of AIDS Antiviral Drugs as Biopharmaceuticals. Adv. Pharmacol. (San Diego) 1995, 6, 389-438, the disclosure of which is incorporated herein by reference. Evaluation of the antiviral activity of the compounds against HIV-1 strain IIIB and HIV-2 strain (ROD) in MT-4 cells was performed using the MTT assay as previously described. See, Pauwels, R.; Balzarini, J.; Baba, M.; Snoeck, R.; Schols, D.; Herdewijn, P.; Desmyter, J.; De Clercq, E. Rapid and Automated Tetrazolium-based Colorimetric Assay for Detection of Anti-HIV Compounds. J. Virol. Methods 1988, 20, 309-321, the disclosure of which is incorporated herein by reference.

In vitro Hydrolytic Stability Study in Rat Plasma. The alkenyldiarylmethanes 1, 3, 4, 8-11, 14-18, 20, 21 and 60-68 (4.3-9.3 mg) (1,1-diphenylethylene (2.1-5.1 mg) or benzophenone (2.1 mg) as internal standard) were tested for their hydrolytic stability, utilizing rat plasma in vitro using methods as previously described. See, Silvestri, M. A.; Nagarajan, M.; De Clercq, E.; Pannecouque, C.; Cushman, M. Design, Synthesis, Anti-HIV Activities, and Metabolic Stabilities of Alkenyldiarylmethane (ADAM) Non-nucleoside Reverse Transcriptase Inhibitors. J. Med. Chem. 2004, 47, 3149-3162, the disclosure of which is incorporated herein by reference.

Biological Results and Discussion. The compounds described herein were evaluated for prevention of the cytopathic effect of HIV-1_{RF} in CEM-SS cells and for cytotoxicity in uninfected CEM-SS cells and MT-4 cells. The biological data are listed in Table 1. The compounds were also tested for their ability to inhibit HIV-1 RT, and the resulting IC₅₀ values are also included in Table 1. Twenty analogues were found to inhibit HIV-1 RT with poly(rC)-oligo(dG) as the template primer with IC₅₀ values ranging from 0.02 to 97.8 μM. Twelve compounds also prevented the cytopathic effect of HIV-1_{RF} with EC₅₀ values ranging from 0.03 to 8.3 μM. Twenty compounds were tested for inhibition of cytopathic effects of both HIV-1_{IIIB} and HIV-2_{ROD} in MT-4 cells, and the resulting EC₅₀ values are also listed in Table 1, along with their cytotoxicities (CC₅₀ values) in uninfected CEM-SS cells and MT-4 cells. Ten compounds displayed EC₅₀ versus HIV-1_{IIIB} between 0.09 to 4.48 μM.

The metabolic stabilities of compounds 1, 3, 4, 8-11, 14-18, 20, 21 and 60-68 in rat plasma were also investigated, and the resulting half-lives of the compounds are also summarized in Table 1. The compounds displayed a range of metabolic stabilities in rat plasma, with half-lives ranging from 0.2 to 5.8 min, except compounds 8, 9, and 68. Compounds 8 and 9, which lack any methyl ester moieties, had not been hydrolyzed at 37° C. after three days. Compound 68, which also lacks any methyl ester moieties had not been hydrolyzed at 37° C. after 1 day.

TABLE 1
Anti-HIV Activities, Cytotoxicities, and Metabolic Stabilities of Compounds
of Formulae I-IV

| | | | | | CC₅₀ (μM)^{c} | | Rat Plasma |
|---|---|---|---|---|---|---|---|
| | IC₅₀ | EC₅₀ (μM)^{b} | | | CEM-SS | MT-4 | tp_{1/2} |
| Comp. | (μM)^{a} | HIV-1_{RF} | HIV-1_{IIIB} | HIV-2_{-ROD} | Cells | Cells | (min ± SD) |
| 1 | 1.0 | 0.25 | 1.0 | NA^{d} | 6.0 | 6.1 | 0.76 ± 0.04 |
| 3 | 1.0 | >100 | >1.05 | >1.05 | 0.49 | 1.05 | 5.76 ± 0.68 |
| 4 | 0.90 | >100 | >33.80 | >35.35 | 0.6 | 83.10 | 0.79 ± 0.10 |
| 7 | 15.70 | 8.30 | NT | NT | 15.0 | NT | NT |
| 8 | 97.850 | >100 | NT^{e} | NT | 14.5 | NT | NH^{f} |
| 9 | >100 | >100 | NT | NT | 26.7 | NT | NH |
| 10 | >100 | >100 | NT | NT | 15.5 | NT | 0.77 ± 0.08 |
| 11 | >100 | >100 | NT | NT | 8.0 | NT | 0.98 ± 0.02 |
| 12 | >100 | >5.0 | >5.51 | >5.89 | >5.0 | 5.70 | NT |
| 13 | >100 | >100 | NT | NT | 16.1 | NT | NT |
| 14 | >100 | >100 | NT | NT | 8.3 | NT | 0.49 ± 0.02 |
| 15 | 55.30 | 7.30 | 2.54 | 10.57 | 9.3 | 112.76 | 4.35 ± 0.41 |
| 16 | 0.84 | >100 | 12.79 | >169.10 | 23.3 | 153.39 | 0.71± 0.01 |
| 17 | 0.75 | 7.1 | 8.55 | >222.96 | 20.2 | 213.45 | 0.44 ± 0.05 |
| 18 | 0.78 | 7.0 | 9.11 | >67.60 | 16.3 | ≧45.15 | 0.42 ± 0.02 |
| 19 | 0.9 | >100 | >5.76 | >5.60 | 2.1 | 5.76 | NT |
| 20 | 5.7 | >100 | >2.05 | >5.63 | 0.8 | 4.69 | 1.16 ± 0.08 |
| 21 | 8.2 | >100 | NT | NT | 13.0 | NT | 0.48 ± 0.06 |
| 22 | >100 | >100 | >35.73 | >43.21 | 16.8 | 39.79 | NT |
| 60 | 0.16 | 0.87 | 1.01 | >28.23 | 9.39 | 28.23 | 1.74 ± 0.00 |
| 61 | 0.93 | 0.53 | 0.25 | >29.72 | 9.47 | 29.72 | 0.19 ± 0.01 |
| 62 | 0.99 | 3.08 | 4.15 | >115 | >13.46 | 115 | 0.34 ± 0.15 |
| 63 | >100 | >100 | 4.36 | >60.03 | 15.0 | 50.95 | 3.06 ± 0.32 |
| 64 | 0.02 | 0.03 | 0.09 | >16.86 | 5.1 | 16.86 | 1.30 ± 0.09 |
| 65 | 49.2 | 1.79 | 4.48 | >36.52 | 11.1 | 36.52 | 0.46 ± 0.03 |
| 66 | 0.5 | 0.62 | 0.22 | >32.52 | 31 | 32.52 | 0.59 ± 0.01 |
| 67 | 0.25 | 0.26 | 0.33 | >7.26 | 2.08 | 7.26 | 1.58 ± 0.13 |
| 68 | 3.6 | >20 | >9.54 | >9.54 | 2.99 | 9.54 | >1440 |

^{a}Inhibitory activity vs HIV-1 reverse transcriptase with poly(rC)•oligo(dG) as the template primer.
^{b}EC₅₀ is the 50% inhibitory concentration for inhibition of cytopathicity of HIV-1_{RF}, HIV-1_{IIIB} or HIV-2_{ROD}.
^{c}The CC₅₀ is the 50% cytotoxic concentration for mock-infected CEM-SS cells or MT-4 cells.
^{d}Not active.
^{e}Not tested.
^{f}Not hydrolyzed.
Data for compounds 3-4, and 7-22 are derived from triplicate tests with the variation of the mean averaging 10%.
Data for compounds 1, and 60-68 represent mean values for at least two separate experiments.

What is claimed is:

1. A compound of the formula 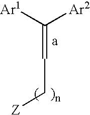
wherein
double bond a is a E-double bond or a Z-double bond;
n is an integer in the range from 1 to about 5;
Ar¹ and Ar² are each independently selected from the group consisting of optionally substituted monocyclic aryl and optionally substituted bicyclic aryl; and Z is a carboxylic acid analog or derivative; provided that when Z is CO₂Me, Ar¹ and Ar² are different from each other.

2. The compound of claim 1 wherein n is 2 or 3.

3. The compound of claim 1 wherein Z is an alkyl ester.

4. The compound of claim 1 wherein Z is a cyclic carbamate.

5. The compound of claim 1 wherein Z is an oxazolidinone.

6. The compound of claim 1 wherein at least one of Ar¹ and Ar² is phenyl substituted with halo, alkyl, alkoxy, haloalkyl, haloalkoxy, alkylthio, hydroxy, nitro, carboxylate and derivatives thereof, cyano, carbamoyl, carboxamido, amino, alkylamino, dialkylamino, alkylalkylamino, sulfonamide, or alkylsulfonylamino, or a combination thereof.

7. The compound of claim 1 wherein at least one of Ar¹ and Ar² is phenyl substituted with halo, alkyl, alkoxy, haloalkyl, haloalkoxy, or cyano, or a combination thereof.

8. The compound of claim 1 wherein at least one of Ar¹ and Ar² is selected from the group consisting of optionally substituted benzisoxazolyl and optionally substituted benzisoxazolinonyl.

9. The compound of claim 1 wherein at least one of Ar¹ and Ar² is selected from the group consisting of optionally substituted benzoxazolyl and optionally substituted benzoxazolinonyl.

10. The compound of claim 1 wherein Ar¹ is selected from the group consisting of 5-fluoro-3-trifluoromethylphenyl, 5-fluoro-2-trifluoromethylphenyl, 5-chloro-2-methoxyphenyl, and 3-cyanophenyl.

11. The compound of claim 1 wherein Ar¹ is 3-cyanophenyl.

12. A pharmaceutical composition comprising the compound of claim 1; and a pharmaceutically acceptable carrier, diluent, or excipient therefor.

13. A method for treating a patient in need of relief from a viral infection, the method comprising the step of administering to the patient a composition comprising the compound of claim 1 in an amount effective to provide relief from the viral infection.

14. The method of claim 13, wherein the composition further comprises a pharmaceutically acceptable carrier, diluent, or excipient.

15. The method of claim 13 wherein the viral infection is acquired immunodeficiency syndrome.

16. The method of claim 13 wherein the viral infection is responsive to inhibition of HIV-1 reverse transcriptase.

17. A process for preparing the compound of claim 1, the process comprising the step of contacting a solution including toluene; a compound of the formula Ar²-L; a metal catalyst; and CsF; with a compound of the formula 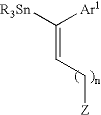
where said contacting step is performed under reactive conditions to prepare a compound of the formula 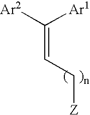
wherein n is an integer in the range from 1 to about 5; Ar¹ and Ar² are each independently selected from optionally substituted monocyclic aryl and optionally substituted bicyclic aryl, L is a leaving group, R is an alkyl group, and Z is a carboxylic acid analog or derivative.

18. A process for preparing a compound of the formula 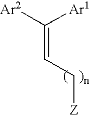
the process comprising the steps of
(a) contacting a solution including toluene; a compound of the formula Ar²-L; a metal catalyst; and CsF; with a compound of the formula 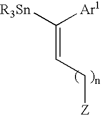
wherein n is an integer in the range from 1 to about 5, Ar¹ and Ar² are each independently selected from optionally substituted monocyclic aryl and optionally substituted bicyclic aryl, L is a leaving group, R is an alkyl group, and Z is a carboxylic acid analog or derivative; and
(b) heating the solution.

19. The compound of claim 8 wherein the optionally substituted benzisoxazolyl or optionally substituted benzisoxazolinonyl is of the formula 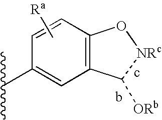
wherein
R^{a} represents 1, 2, or 3 substituents each independently selected from the group consisting of halo, alkyl, alkoxy, haloalkyl, haloalkoxy, alkylthio, hydroxy, nitro, carboxylate and derivatives thereof, cyano, carbamoyl, carboxamido, amino, alkylamino, dialkylamino, alkylalkylamino, sulfonamide, and alkylsulfonylamino; and
one of bond b or bond c is a double bond, and the other of bond b or bond c is a single bond; and R^{b} and R^{c} are each an optionally substituted alkyl; providing that when bond b is a double bond, R^{b} is absent; and when bond c is a double bond, R^{c} is absent.

20. The compound of claim 9 wherein the optionally substituted benzoxazolyl or optionally substituted benzoxazolinonyl is of the formula 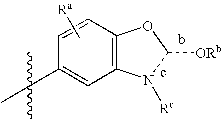
wherein
R^{a} represents 1, 2, or 3 substituents each independently selected from the group consisting of halo, alkyl, alkoxy, haloalkyl, haloalkoxy, alkylthio, hydroxy, nitro, carboxylate and derivatives thereof, cyano, carbamoyl, carboxamido, amino, alkylamino, dialkylamino, alkylalkylamino, sulfonamide, and alkylsulfonylamino; and
one of bond b or bond c is a double bond, and the other of bond b or bond c is a single bond; and R^{b} and R^{c} are each an optionally substituted alkyl; providing that when bond b is a double bond, R^{b} is absent; and when bond c is a double bond, R^{c} is absent.
